(19) United States
(12) Patent Application Publication
(10) Pub. No.: US 2025/0019424 A1
(43) Pub. Date: Jan. 16, 2025

(54) VHH POLYPEPTIDES THAT BIND TO CLOSTRIDIUM DIFFICILE TOXIN B AND METHODS OF USE THEREOF

(71) Applicant: Trustees of Tufts College, Medford, MA (US)

(72) Inventor: Charles B. SHOEMAKER, Medford, MA (US)

(73) Assignee: Trustees of Tufts College, Medford, MA (US)

(21) Appl. No.: 18/787,648

(22) Filed: Jul. 29, 2024

Related U.S. Application Data
(63) Continuation of application No. 18/013,195, filed on Dec. 27, 2022, now Pat. No. 12,077,576.
(60) Provisional application No. 63/047,436, filed on Jul. 2, 2020.

(51) Int. Cl. C07K 16/12; A61K 39/00; A61P 31/04; C07K 14/47

(52) U.S. Cl. CPC C07K 16/1282; A61P 31/04; C07K 14/47; A61K 2039/505

(57) ABSTRACT

Polypeptide products, methods, pharmaceutical compositions, and kits are provided for treating a subject exposed to, or at risk for exposure to, C. difficile microbial pathogens and toxin B produced by C. difficile (TcdB) pathogens. The methods, compositions and kits include a single domain, anti-TcdB VHH polypeptide (antibody), or toxin B binding portion thereof, that specifically binds to and/or neutralizes TcdB and treats or prevents illness and disease associated with C. difficile infection and TcdB intoxication. The anti-TcdB VHHs, or toxin B binding portion thereof, may be recombinantly produced.

### CROSS REFERENCE TO RELATED APPLICATIONS

This application is a continuation of U.S. application Ser. No. 18/013,195, filed Dec. 27, 2022, which is the U.S. National Stage Application, pursuant to 35 U.S.C. § 371, of International PCT Application No. PCT/US2021/039798, filed Jun. 30, 2021, designating the United States and published in English, which claims priority to and benefit of U.S. Provisional Application No. 63/047,436, filed Jul. 2, 2020, the contents of all of which are incorporated by reference herein in their entirety.

### SEQUENCE LISTING

This application contains a Sequence Listing which has been submitted electronically in compliance with ST.26 format and is hereby incorporated by reference in its entirety. The Sequence Listing, created on Jul. 29, 2024, is named 167774-012403/US/CON_SL.xml and is 236,147 bytes in size.

### BACKGROUND

Clostridium difficile (C. difficile or C. diff.) microorganisms are enteric pathogens that infect many people worldwide causing loss of life and great economic cost. C. diff. is the most prevalent cause of hospital-acquired infectious diarrhea and life-threatening colitis worldwide. Yearly costs in the United States alone are estimated at $4.8 billion. C. diff. infection (CDI) typically results from an unchecked growth and colonization of the pathogenic microorganism following anti-microbial therapies. The primary cause of CDI pathology is the production and release of exotoxins, especially toxin B, by the C. diff. pathogen. The toxin wreaks havoc with organs in the gastrointestinal system of infected individuals and severely disrupts the normal microflora that maintain gut health and homeostasis.

The emergence of hypervirulent drug-resistant C. diff. microorganisms has resulted in a global CDI epidemic that is largely due to high level production of C. diff. exotoxins, which cause CDI pathology.

Binding molecules, other than classical antibodies, that specifically bind to C. diff. toxins are needed for the treatment of disease and intoxication caused by C. diff. infection. The production and storage of classical antibodies involve labor-intensive and costly processes. In fact, the development of a single antibody therapeutic agent frequently requires years of clinical study and testing. Often, multiple, different therapeutic antibodies are necessary for the effective treatment of patients exposed to C. diff. or an outbreak of infection caused by this microorganism and its toxins. In addition, the shelf-life and stability of stored, classical antibodies are often relatively short (e.g., weeks or months), thus requiring the production of new batches of antibodies to replace those that have expired due to degradation or other adverse effects on the integrity of antibody proteins.

Thus, there is a need for new and cost effective therapeutic agents for treating the disease-causing microorganism C. diff., as well as for treating subjects afflicted with CDI and its serious symptoms resulting from the toxins produced by C. diff. A profound need also exists for alternative therapeutics that are easier to develop and produce and have a longer shelf life for use against disease-causing C. diff. The present products, compositions and methods provide a solution to these needs.

### SUMMARY

Described herein are VHH-based polypeptides (antibodies) that specifically bind to toxin B (also termed TcdB) produced by Clostridium difficile (C. difficile) microorganisms. The VHH polypeptides described herein are monomeric, single chain antibodies that bind to TcdB, i.e., anti-TcdB VHHs or anti-TcdB VHH molecules. In some embodiments, the anti-TcdB VHHs both bind to and neutralize TcdB produced by C. difficile. In some embodiments, the anti-TcdB VHHs bind to and neutralize TcdB in vitro and/or in vivo.

In an aspect, a polypeptide that specifically binds to C. difficile toxin B (TcdB), or a TcdB-binding portion thereof, is provided, wherein the polypeptide comprises three complementarity determining regions (CDRs), CDR1, CDR2 and CDR3, and four VHH framework regions (FRs), FR1, FR2, FR3 and FR4, with the general structure FR1-CDR1-FR2-CDR2-FR3-CDR3-FR4,
wherein CDR1 comprises amino acid sequence GSVYTF (SEQ ID NO: 25); CDR2 comprises amino acid sequence SGGTITK (SEQ ID NO: 26); and CDR3 comprises amino acid sequence NAGDTIAQAMGTRRFPFDR (SEQ ID NO: 27);
wherein CDR1 comprises amino acid sequence GTSFPRNY (SEQ ID NO: 28); CDR2 comprises amino acid sequence SHDGNVE (SEQ ID NO: 29); and CDR3 comprises amino acid sequence KLVTLRRDEY (SEQ ID NO: 30);
wherein CDR1 comprises amino acid sequence RFSLINYA (SEQ ID NO: 31); CDR2 comprises amino acid sequence TSGGATY (SEQ ID NO: 32); and CDR3 comprises amino acid sequence AAGPYSRTLVSRWKVGDGMEY (SEQ ID NO: 33);
wherein CDR1 comprises amino acid sequence GFTSNSYY (SEQ ID NO: 34); CDR2 comprises amino acid sequence SSSGGSPN (SEQ ID NO: 35); and CDR3 comprises amino acid sequence AASKFPLTTMASNRYHY (SEQ ID NO: 36);
wherein CDR1 comprises amino acid sequence GRGPGINV (SEQ ID NO: 37); CDR2 comprises amino acid sequence QTGGTTN (SEQ ID NO: 38); and CDR3 comprises amino acid sequence YLKKWRDEY (SEQ ID NO: 39);
wherein CDR1 comprises amino acid sequence GSSFSMNV (SEQ ID NO: 40); CDR2 comprises amino acid sequence RSDGITN (SEQ ID NO: 41); and CDR3 comprises amino acid sequence FHGRARTGNNADLGS (SEQ ID NO: 42);
wherein CDR1 comprises amino acid sequence GRLSERIFMIST (SEQ ID NO: 43); CDR2 comprises amino acid sequence SRLGRAN (SEQ ID NO: 44); and CDR3 comprises amino acid sequence NLKPFVDNYR (SEQ ID NO: 45);
wherein CDR1 comprises amino acid sequence GITFSNVA (SEQ ID NO: 46); CDR2 comprises amino acid sequence STGGSSTS (SEQ ID NO: 47); and CDR3 comprises amino acid sequence VKGPKYSATIRRPE (SEQ ID NO: 48);
wherein CDR1 comprises amino acid sequence GFNFSVQI (SEQ ID NO: 49); CDR2 comprises amino acid sequence STGGASKS (SEQ ID NO: 50); and CDR3 comprises amino acid sequence SKGPRTWINSSPR (SEQ ID NO: 51);
wherein CDR1 comprises amino acid sequence GTAFSLDT (SEQ ID NO: 52); CDR2 comprises amino acid sequence SSSGASN (SEQ ID NO: 53); and CDR3 comprises amino acid sequence YRGRVRGVWPLDSGMMY (SEQ ID NO: 54);
wherein CDR1 comprises amino acid sequence GSILSS (SEQ ID NO: 55); CDR2 comprises amino acid sequence SRTGATD (SEQ ID NO: 56); and CDR3 comprises amino acid sequence NAGLGMGDPRRPGPW (SEQ ID NO: 57); or
wherein CDR1 comprises amino acid sequence ERNPGINA (SEQ ID NO: 58); CDR2 comprises amino acid sequence WQTGGSLS (SEQ ID NO: 59); and CDR3 comprises amino acid sequence YLKKWRDQY (SEQ ID NO: 60). In an embodiment, the four VHH FRs are camelid VHH FRs. In an embodiment, the polypeptide neutralizes C. difficile toxin B (TcdB) activity. In an embodiment, the polypeptide is a camelid-derived single domain anti-TcdB VHH antibody. In an embodiment, the polypeptide is in the form of a dimer or multimer. In an embodiment, the polypeptide comprises one or more epitope tag sequences specifically bindable by an anti-epitope tag antibody or binding portion thereof.

In an aspect, a polypeptide that specifically binds to C. difficile toxin B (TcdB) is provided, wherein the polypeptide or a TcdB-binding portion thereof has at least 85% amino acid sequence identity to a sequence selected from the group consisting of SEQ ID NOs: 1, 3, 5, 7, 9, 11, 13, 15, 17, 19, 21 and 23.

In an aspect, a polypeptide that specifically binds to C. difficile toxin B (TcdB) is provided, wherein the polypeptide or a TcdB-binding portion thereof has at least 90% amino acid sequence identity to a sequence selected from the group consisting of SEQ ID NOs: 1, 3, 5, 7, 9, 11, 13, 15, 17, 19, 21 and 23.

In an aspect, a polypeptide that specifically binds to C. difficile toxin B (TcdB) is provided, wherein the polypeptide or an antigen binding portion thereof has at least 95% amino acid sequence identity to a sequence selected from the group consisting of SEQ ID NOs: 1, 3, 5, 7, 9, 11, 13, 15, 17, 19, 21 and 23.

In an aspect, a polypeptide that specifically binds to C. difficile toxin B (TcdB) wherein the polypeptide or a TcdB-binding portion thereof has at least 98% amino acid sequence identity to a sequence selected from the group consisting of SEQ ID NOs: 1, 3, 5, 7, 9, 11, 13, 15, 17, 19, 21 and 23.

In an embodiment of any of the above-delineated aspects and embodiments, conservative amino acid substitutions in the polypeptide comprise the at least 85%, at least 90%, at least 95%, or at least 98% amino acid sequence identity.

In an aspect, a polypeptide that specifically binds to C. difficile toxin B (TcdB) is provided, wherein the polypeptide or a TcdB-binding portion thereof comprises a sequence selected from the group consisting of SEQ ID NOs: 1, 3, 5, 7, 9, 11, 13, 15, 17, 19, 21 and 23. In an aspect, a polypeptide that specifically binds to C. difficile toxin B (TcdB) wherein the polypeptide or a TcdB-binding portion thereof consists of a sequence selected from the group consisting of SEQ ID NOs: 1, 3, 5, 7, 9, 11, 13, 15, 17, 19, 21 and 23. In an embodiment of the above-delineated aspects, the polypeptide neutralizes TcdB activity. In an embodiment, the polypeptide is a camelid-derived single domain VHH antibody (VHH). In an embodiment, the polypeptide is in the form of a dimer or multimer.

In an embodiment of the above-delineated aspects, the polypeptide comprises one or more epitope tag sequences specifically bindable by an anti-epitope tag antibody or binding portion thereof. In an embodiment, the one or more epitope tag sequences comprises at least one of DELGPRLMGK (SEQ ID NO: 61) or GAPVPYPDPLEPR (SEQ ID NO: 62).

In another aspect, a dimeric or multimeric polypeptide comprising two or more anti-TcdB VHH polypeptides is provided, which comprises a sequence selected from the group consisting of SEQ ID NOs: 1, 3, 5, 7, 9, 11, 13, 15, 17, 19, 21 and 23, or TcdB binding regions thereof, wherein the two or more anti-TcdB VHH polypeptides, or TcdB binding regions, are joined with one or more linker peptides. In an embodiment, the one or more linker peptides is selected from GGGGS (SEQ ID NO: 63); GGGGSGGGGSGGGGS (SEQ ID NO: 64), or a functional portion thereof; EPKTPKPQGGGGSGGGGSGGGGSQGVQSQVQLVE (SEQ ID NO: 65); EPKTPKPQ (SEQ ID NO: 66); or a combination thereof. In an embodiment, the dimeric or multimeric polypeptide comprises one or more epitope tag sequences specifically bindable by an anti-epitope tag antibody or binding portion thereof. In an embodiment, the one or more epitope tag sequences comprises at least one of DELGPRLMGK (SEQ ID NO: 61) or GAPVPYPDPLEPR (SEQ ID NO: 62). In an embodiment, the polypeptide is dimeric and comprises two anti-TcdB VHH polypeptides. In an embodiment of the dimeric polypeptide, the two anti-TcdB VHH polypeptides are the same. In an embodiment of the dimeric polypeptide, the two anti-TcdB VHH polypeptides are different. In an embodiment, the polypeptide is multimeric and comprises at least three anti-TcdB VHH polypeptides. In an embodiment, the multimeric polypeptide comprises at least four anti-TcdB VHH polypeptides. In an embodiment, the multimeric polypeptide comprises three or four anti-TcdB VHH polypeptides. In an embodiment, the anti-TcdB VHH polypeptides of the multimeric polypeptide are the same. In an embodiment, the anti-TcdB VHH polypeptides of the multimeric polypeptide are different. In an embodiment, the anti-TcdB VHH polypeptides of the multimeric polypeptide are a combination of the same and different anti-TcdB VHH polypeptides.

In an aspect, an isolated polynucleotide encoding the polypeptide of the above-delineated aspects and embodiments is provided.

In an aspect, an isolated polynucleotide encoding the dimeric or multimeric polypeptide of the above-delineated aspects and embodiments is provided.

In an aspect, an isolated polynucleotide having at least 90% sequence identity to a nucleic acid sequence selected from the group consisting of SEQ ID NOs: 2, 4, 6, 8, 10, 12, 14, 16, 18, 20, 22 and 24 is provided.

In an aspect, an isolated polynucleotide having at least 95% sequence identity to a nucleic acid sequence selected from the group consisting of SEQ ID NOs: 2, 4, 6, 8, 10, 12, 14, 16, 18, 20, 22 and 24 is provided.

In an aspect, an isolated polynucleotide having at least 98% sequence identity to a nucleic acid sequence selected from the group consisting of SEQ ID NOs: 2, 4, 6, 8, 10, 12, 14, 16, 18, 20, 22 and 24 is provided.

In an aspect, an isolated polynucleotide comprising a sequence selected from the group consisting of SEQ ID NOs: 2, 4, 6, 8, 10, 12, 14, 16, 18, 20, 22 and 24 is provided.

In an aspect, an isolated polynucleotide consisting of a sequence selected from the group consisting of SEQ ID NOs: 2, 4, 6, 8, 10, 12, 14, 16, 18, 20, 22 and 24 is provided.

In an aspect, an isolated polynucleotide comprising a nucleic acid sequence encoding an anti-TcdB VHH of any one of SEQ ID NOs: 1, 3, 5, 7, 9, 11, 13, 15, 17, 19, 21, or 23 is provided.

In an aspect, an isolated polynucleotide having at least 85% sequence identity to a nucleic acid sequence encoding an anti-TcdB VHH of any one of SEQ ID NOs: 1, 3, 5, 7, 9, 11, 13, 15, 17, 19, 21, or 23 is provided.

In an aspect, an isolated polynucleotide having at least 90% sequence identity to a nucleic acid sequence encoding an anti-TcdB VHH of any one of SEQ ID NOs: 1, 3, 5, 7, 9, 11, 13, 15, 17, 19, 21, or 23 is provided.

In an aspect, an isolated polynucleotide having at least 95% sequence identity to a nucleic acid sequence encoding an anti-TcdB VHH of any one of SEQ ID NOs: 1, 3, 5, 7, 9, 11, 13, 15, 17, 19, 21, or 23 is provided.

In an aspect, an isolated polynucleotide having at least 98% sequence identity to a nucleic acid sequence encoding an anti-TcdB VHH of any one of SEQ ID NOs: 1, 3, 5, 7, 9, 11, 13, 15, 17, 19, 21, or 23 is provided.

In an aspect, a vector comprising a nucleic acid molecule that encodes a polypeptide as delineated in the above aspects is provided.

In an aspect, a vector comprising a nucleic acid molecule that encodes the dimeric or multimeric polypeptide as delineated in the above aspects is provided.

In an aspect, a vector comprising the isolated polynucleotide as delineated in the above aspects is provided. In an embodiment of the above-delineated aspects, the vector is an expression vector. In an embodiment, the expression vector is a viral or non-viral expression vector.

In an aspect, a host cell comprising the vector of any of the above-delineated aspects and embodiments is provided.

In an aspect, a non-naturally occurring Spirulina cyanobacterium (“Spirulina cyanobacterium”) expressing the polypeptide of any of the above-delineated aspects and embodiments is provided. In an aspect, a non-naturally occurring Spirulina cyanobacterium comprising the isolated polynucleotide of any of the above-delineated aspects and embodiments is provided. In an embodiment, the Spirulina cyanobacterium is a stable transformant comprising at least one introduced targeted nucleotide mutation in its genome. In an embodiment, the Spirulina cyanobacterium is Arthrospira platensis or Arthrospira maxima. In an embodiment, the Spirulina cyanobacterium is Arthrospira platensis NIES-39 or Arthrospira sp. PCC 8005. In an embodiment, the Spirulina cyanobacterium is provided for treating disease, pathology and/or symptoms thereof caused by C. difficile infection or intoxication (CDI). In an embodiment, the Spirulina cyanobacterium is administered orally to a subject in need thereof. In an embodiment, the Spirulina cyanobacterium of claim 54, which is administered enterically to a subject in need thereof.

In an aspect, a probiotic microorganism molecularly engineered to contain the isolated polynucleotide of any of the above-delineated aspects and embodiments is provided. In an embodiment, the probiotic microorganism is E. coli strain Nissle.

In an aspect, an enteric coated particle comprising the polypeptide of any of the above-delineated aspects or embodiments, or the isolated polynucleotide of any of the above-delineated aspects and embodiments is provided. In an embodiment, the particle comprises a protease resistant polypeptide, or a TcdB binding fragment thereof, of any of the above-delineated aspects or embodiments.

In an aspect, a pharmaceutical composition comprising an effective amount of the polypeptide of any of the above-delineated aspects or embodiments, or a TcdB binding fragment thereof, and a pharmaceutically acceptable excipient, carrier, or diluent is provided.

In an aspect, a pharmaceutical composition comprising an effective amount of the isolated polynucleotide of any of the above-delineated aspects or embodiments, and a pharmaceutically acceptable excipient, carrier, or diluent is provided.

In an aspect, a pharmaceutical composition comprising an effective amount of the probiotic microorganism of the above-delineated aspects and embodiments, or of the particle of the above-delineated aspects and embodiments, and a pharmaceutically acceptable excipient, carrier, or diluent is provided.

In an aspect, a pharmaceutical composition comprising an effective amount of the non-naturally occurring Spirulina cyanobacterium of any of the above-delineated aspects or embodiments, and a pharmaceutically acceptable excipient, carrier, or diluent is provided.

In an aspect, a method of reducing, ameliorating, alleviating, abating, or eradicating intoxication of a cell by C. difficile toxin B (TcdB) is provided, in which the method comprises contacting the cell with an effective amount of the polypeptide of any one of the above-delineated aspects and embodiments, thereby reducing, ameliorating, alleviating, abating, or eradicating intoxication of the cell by TcdB.

In an aspect, a method of reducing, ameliorating, alleviating, abating, or eradicating intoxication of a cell by C. difficile toxin B (TcdB) is provided, in which the method comprises contacting the cell with an effective amount of the isolated polynucleotide of any one of the above-delineated aspects and embodiments; the non-naturally occurring Spirulina cyanobacterium of any one of the above-delineated aspects and embodiments; or the probiotic microorganism of the above-delineated aspects and embodiments, thereby reducing, ameliorating, alleviating, abating, or eradicating intoxication of the cell by TcdB.

In an embodiment of the above methods, the cell is an enteric cell of the gastrointestinal (GI) tract or the gut. In an embodiment of the above methods, the cell is in vitro. In an embodiment of the above methods, the cell is in vivo.

In an aspect, a method of treating C. difficile infection (CDI) and/or the symptoms thereof in a subject is provided, in which the method comprises administering to a subject in need thereof an effective amount of the polypeptide of any one of the above-delineated aspects and embodiments; the isolated polynucleotide of any one of the above-delineated aspects and embodiments; the non-naturally occurring Spirulina cyanobacterium of any one of the above-delineated aspects and embodiments; the probiotic microorganism of the above-delineated aspects and embodiments, or a pharmaceutical composition thereof, thereby treating CDI and/or the symptoms thereof in the subject.

In an aspect, a method of reducing the severity of C. difficile infection (CDI) or intoxication by C. difficile and/or the symptoms thereof in a subject who has, who is susceptible to, or who is at risk of CDI or intoxication by C. difficile is provided, in which the method comprises administering to a subject in need thereof an effective amount of the polypeptide of any one of the above-delineated aspects and embodiments; the isolated polynucleotide of any one of the above-delineated aspects and embodiments; the non-naturally occurring Spirulina cyanobacterium of any one of the above-delineated aspects and embodiments; the probiotic microorganism of the above-delineated aspects and embodiments, or a pharmaceutical composition thereof, thereby reducing the severity of C. difficile infection (CDI) or intoxication by C. difficile and/or the symptoms thereof in the subject.

In an aspect, a method of preventing C. difficile infection (CDI) and/or the symptoms thereof in a subject is provided, in which the method comprises administering to a subject in need thereof an effective amount of the polypeptide of any one of the above-delineated aspects and embodiments; the isolated polynucleotide of any one of the above-delineated aspects and embodiments; the non-naturally occurring Spirulina cyanobacterium of any one of the above-delineated aspects and embodiments; the probiotic microorganism of the above-delineated aspects and embodiments, or a pharmaceutical composition thereof, thereby preventing CDI and/or the symptoms thereof in the subject.

In an aspect, a method of treating or preventing C. difficile infection (CDI) and/or the symptoms thereof in a subject is provided, in which the method comprises administering to a subject in need thereof an effective amount of the particle of the above-delineated aspects and embodiments, or a pharmaceutical composition thereof, thereby treating CDI and/or the symptoms thereof in the subject.

In an embodiment of the above-delineated methods of reducing the severity or, preventing, or treating or preventing CDI, the CDI and the symptoms thereof cause one or more of C. difficile-associated diarrhea (CDAD), pseudomembranous colitis (PMC), bowel inflammation, enterocytic detachment, alteration, disruption, or elimination of natural intestinal microflora, and/or paralytic ileus. In another embodiment, the methods further comprise administering to the subject an anti-epitope tag antibody that specifically binds to an epitope tag, if present, and facilitates clearance of a complex of TcdB bound to the anti-TcdB VHH polypeptide from the subject.

In another aspect, a polypeptide that specifically binds to and neutralizes C. difficile toxin B (TcdB), or a TcdB-binding portion thereof, is provided, wherein the polypeptide comprises three complementarity determining regions (CDRs), CDR1, CDR2 and CDR3, and four VHH framework regions (FRs), FR1, FR2, FR3 and FR4, with the general structure FR1-CDR1-FR2-CDR2-FR3-CDR3-FR4,
wherein CDR1 comprises amino acid sequence RFSLINYA (SEQ ID NO: 31); CDR2 comprises amino acid sequence TSGGATY (SEQ ID NO: 32); and CDR3 comprises amino acid sequence AAGPYSRTLVSRWKVGDGMEY (SEQ ID NO: 33);
wherein CDR1 comprises amino acid sequence GFTSNSYY (SEQ ID NO: 34); CDR2 comprises amino acid sequence SSSGGSPN (SEQ ID NO: 35); and CDR3 comprises amino acid sequence AASKFPLTTMASNRYHY (SEQ ID NO: 36);
wherein CDR1 comprises amino acid sequence GRGPGINV (SEQ ID NO: 37); CDR2 comprises amino acid sequence QTGGTTN (SEQ ID NO: 38); and CDR3 comprises amino acid sequence YLKKWRDEY (SEQ ID NO: 39);
wherein CDR1 comprises amino acid sequence GSSFSMNV (SEQ ID NO: 40); CDR2 comprises amino acid sequence RSDGITN (SEQ ID NO: 41); and CDR3 comprises amino acid sequence FHGRARTGNNADLGS (SEQ ID NO: 42);
wherein CDR1 comprises amino acid sequence GRLSERIFMIST (SEQ ID NO: 43); CDR2 comprises amino acid sequence SRLGRAN (SEQ ID NO: 44); and CDR3 comprises amino acid sequence NLKPFVDNYR (SEQ ID NO: 45);
wherein CDR1 comprises amino acid sequence GITFSNVA (SEQ ID NO: 46); CDR2 comprises amino acid sequence STGGSSTS (SEQ ID NO: 47); and CDR3 comprises amino acid sequence VKGPKYSATIRRPE (SEQ ID NO: 48);
wherein CDR1 comprises amino acid sequence GFNFSVQI (SEQ ID NO: 49); CDR2 comprises amino acid sequence STGGASKS (SEQ ID NO: 50); and CDR3 comprises amino acid sequence SKGPRTWINSSPR (SEQ ID NO: 51);
wherein CDR1 comprises amino acid sequence GSILSS (SEQ ID NO: 55); CDR2 comprises amino acid sequence SRTGATD (SEQ ID NO: 56); and CDR3 comprises amino acid sequence NAGLGMGDPRRPGPW (SEQ ID NO: 57); or
wherein CDR1 comprises amino acid sequence ERNPGINA (SEQ ID NO: 58); CDR2 comprises amino acid sequence WQTGGSLS (SEQ ID NO: 59); and CDR3 comprises amino acid sequence YLKKWRDQY (SEQ ID NO: 60). In an embodiment, the four VHH FRs are camelid VHH FRs.

In an aspect, a polypeptide that specifically binds to and neutralizes C. difficile toxin B (TcdB) is provided, wherein the polypeptide or a TcdB-binding portion thereof has at least 90% sequence identity to a sequence selected from the group consisting of SEQ ID NOs: 5, 7, 9, 11, 13, 15, 17, 21 and 23.

In an aspect, a polypeptide that specifically binds to and neutralizes C. difficile toxin B (TcdB) is provided, wherein the polypeptide or a TcdB-binding portion thereof has at least 95% sequence identity to a sequence selected from the group consisting of SEQ ID NOs: 5, 7, 9, 11, 13, 15, 17, 21 and 23 is provided.

In an aspect, a polypeptide that specifically binds to and neutralizes C. difficile toxin B (TcdB) is provided, wherein the polypeptide or an antigen binding portion thereof comprises a sequence selected from the group consisting of SEQ ID NOs: 5, 7, 9, 11, 13, 15, 17, 21 and 23.

In an embodiment of the above-delineated aspects of the polypeptide, conservative amino acid substitutions in the polypeptide comprise the at least 90% or the at least 95% amino acid sequence identity. In an embodiment, the polypeptide is a camelid-derived single domain anti-TcdB VHH antibody. In an embodiment, the polypeptide is in the form of a dimer or multimer. In an embodiment, the polypeptide comprises one or more epitope tag sequences specifically bindable by an anti-epitope tag antibody or binding portion thereof.

In an aspect, an isolated polynucleotide encoding the polypeptide of the above-delineated aspects and embodiments is provided.

In an aspect, a pharmaceutical composition comprising an effective amount of the polypeptide of any of the above-delineated aspects and embodiments, or a TcdB-binding fragment thereof, or an effective amount of the above-delineated isolated polynucleotide, and a pharmaceutically acceptable excipient, carrier, or diluent is provided.

In an aspect, a non-naturally occurring Spirulina cyanobacterium expressing the polypeptide of any one of the above-delineated aspects and embodiments, or comprising the above-delineated isolated polynucleotide and embodiments thereof is provided.

In an aspect, a probiotic microorganism molecularly engineered to contain the above-delineated isolated polynucleotide and embodiments thereof, optionally wherein the microorganism is E. coli strain Nissle, is provided.

In an aspect, an enteric coated particle comprising the polypeptide of any one of the above-delineated aspects and embodiments or the above-delineated isolated polynucleotide and embodiments thereof is provided. In an embodiment, the polypeptide or the TcdB-binding fragment thereof, is protease resistant.

In an aspect, a method of treating or preventing C. difficile infection (CDI) and/or the symptoms thereof in a subject is provided, wherein the method comprises administering to a subject in need thereof an effective amount of the above-delineated pharmaceutical composition, thereby treating CDI and/or the symptoms thereof in the subject.

In an aspect, a method of treating or preventing C. difficile infection (CDI) and/or the symptoms thereof in a subject is provided, in which the method comprises administering to a subject in need thereof an effective amount of the above-delineated non-naturally occurring Spirulina cyanobacterium, thereby treating CDI and/or the symptoms thereof in the subject.

In an aspect, a kit comprising the polypeptide of any one of the above-delineated aspects or embodiments, or a pharmaceutical composition thereof, is provided for treating or protecting against disease or intoxication and/or the symptoms thereof caused by C. difficile toxin B.

In an aspect, a kit comprising the isolated polynucleotide of any one of the above-delineated aspects or embodiments, or a pharmaceutical composition thereof, is provided for treating or protecting against disease or intoxication and/or the symptoms thereof caused by C. difficile toxin B.

In an aspect, a kit comprising the non-naturally occurring Spirulina cyanobacterium of the above-delineated aspects and embodiments; the probiotic microorganism of the above-delineated aspects and embodiments; the particle of the above-delineated aspects and embodiments, or a pharmaceutical composition thereof, is provided for treating or protecting against disease or intoxication and/or the symptoms thereof caused by C. difficile toxin B.

In an embodiment, the kit of any one of the above-delineated aspects further comprises instructions for use.

In an aspect, a polypeptide that specifically binds to C. difficile toxin B (TcdB), or a TcdB-binding portion thereof, is provided, wherein the polypeptide comprises three complementarity determining regions (CDRs), CDR1, CDR2 and CDR3, and four VHH framework regions (FRs), FR1, FR2, FR3 and FR4, with the general structure FR1-CDR1-FR2-CDR2-FR3-CDR3-FR4,
wherein CDR1 comprises 8 amino acids, X1-X8,, wherein X1 is G or E; X2 is R or A; X3 is G or N; X4 is P or L; X₅ is G; X6 is I; X7 is N; and X8 is V or A;
wherein CDR2 comprises 7 amino acids, X1-X7, wherein X1 is Q; X2 is T; X3 is G; X4 is G; X₅ is T, R, or S; X6 is T or L; and X7is N or S; and
wherein CDR3 comprises 9 amino acid, X1-X9, wherein X1 is Y; X2 is L or Q; X3 is K; X4 is K or R; X₅ is W; X6 is R; X7 is D or N; X8 is E, Q, D, or R; and X9 is Y. In an embodiment, the four VHH FRs are camelid VHH FRs. In an embodiment, the polypeptide neutralizes C. difficile toxin B (TcdB) activity. In an embodiment, the polypeptide is a camelid-derived single domain anti-TcdB VHH antibody. In an embodiment, the polypeptide is in the form of a dimer or multimer. In an embodiment, the polypeptide or dimeric or multimeric form thereof comprises one or more epitope tag sequences specifically bindable by an anti-epitope tag antibody or binding portion thereof.

In an aspect, a method of reducing the severity of C. difficile infection (CDI) or intoxication by C. difficile and/or the symptoms thereof in a subject who has, who is susceptible to, or who is at risk of CDI or intoxication by C. difficile, wherein the method comprises administering to a subject in need thereof an effective amount of the above-delineated polypeptide and embodiments thereof, or a pharmaceutical composition thereof, thereby reducing the severity of C. difficile infection (CDI) or intoxication by C. difficile and/or the symptoms thereof in the subject. In an embodiment of the method, the polypeptide or the pharmaceutical composition thereof is administered to the subject prior or subsequent to infection by C. difficile.

In an embodiment of any of the above-delineated methods and embodiments thereof, the polypeptide, any composition or pharmaceutical composition thereof, is administered to the subject prior or subsequent to infection by C. difficile. In another embodiment of any of the above-delineated methods and embodiments thereof, the subject is to receive or has received antibiotics or a course of antibiotics to treat a non-C. difficile bacterial infection.

### Definitions

Unless defined otherwise, all technical and scientific terms used herein have the meaning commonly understood by a person skilled in the art to which the described aspects and embodiments belongs. The following references provide one of skill with a general definition of many of the terms used in the aspects and embodiments described herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). The following terms have the meanings ascribed to them below, unless specified otherwise.

In this application, the use of the singular includes the plural unless specifically stated otherwise. It must be noted that, as used in the specification, the singular forms “a.” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in the specification and claim(s) herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions and products of the present disclosure can be used to achieve methods of the present disclosure.

Unless specifically stated or obvious from context, as used herein, the term “about” or “approximately” means within an acceptable error range for the particular value as determined by one of ordinary skill in the art, which will depend, in part, on how the value is measured or determined, i.e., the limitations of the measurement system. For example, “about” can mean within 1 standard deviation or more than 1 standard deviation, e.g., 2 standard deviations of the mean, as typically practiced in the art. Alternatively, and without intending to be limiting, “about” can mean a range of up to 20%, up to 10%, up to 5%, up to 2%, or up to 1% of a given value. Alternatively, and particularly for biological systems or processes, the term can mean within an order of magnitude, e.g., within 5-fold, within 3-fold, within 2.5-fold, or within 2-fold of a value. Where particular values are described in the application and claims, unless otherwise stated, the term “about” means within an acceptable error range for the particular value.

Reference herein to “some embodiments,” “an embodiment,” “one embodiment” or “other embodiments” means that a particular feature, structure, or characteristic described in connection with the embodiments is included in at least some embodiments, but not necessarily all embodiments, of the disclosure.

By “agent” is meant any small molecule chemical compound, antibody, nucleic acid molecule, or polypeptide (e.g., antibody or VHH antibody), or fragments thereof.

By “ameliorate” is meant decrease, reduce, diminish, suppress, attenuate, arrest, or stabilize the development or progression of a disease or pathology.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antibody” is meant any immunoglobulin polypeptide, or fragment thereof, having immunogen or antigen binding ability. Antibody structure is well known in the art. Briefly, the variable (V) regions or domains of antibody heavy (H) and light (L) chains contain Complementarity-Determining Regions (CDRs), which bind to specific antigens or immunogens (e.g., protein antigens or immunogens). CDRs are situated within framework (FR) sequences of the V regions of the heavy (V_{H}) and light chains (V_{L}) of an antibody. CDRs are the most variable parts of antibodies and are critical components in the diversity of antigen specificities of antibodies produced by B lymphocytes. In general, three CDRs (CDR1, CDR2 and CDR3) are arranged consecutively in a V domain of an antibody. Because a VHH, such as a camelid VHH, is essentially a single chain antibody polypeptide, it contains three CDRs that bind to an antigen or target protein such as a toxin (e.g., TcdB) in the context of four framework (FR) regions, as follows: FR1-CDR1-FR2-CDR2-FR3 -CDR3-FR4. Thus, as would be appreciated by the skilled practitioner in the art, in a VHH polypeptide sequence, FR1 comprises the amino acids positioned to the left of CDR1; FR2 comprises the amino acids positioned between CDR1 and CDR2; FR3 comprises the amino acids positioned between CDR2 and CDR3; and FR4 comprises the amino acids positioned to the right of CDR3. Because most of the sequence variability associated with immunoglobulins and antigen binding is found in the CDRs, these regions are sometimes referred to as hypervariable regions. Typically, CDR1, CDR2 and CDR3 of VHHs contribute to and/or do not interfere with antigen binding. The CDRs of a number of anti-TcdB VHHs described herein are shown, for example, in Table 1, FIGS. 5A-5C and FIGS. 7A and 7B. The FRs of a number of anti-TcdB VHHs described herein are shown, for example, in FIG. 6B.

A “chimeric antibody” refers to an antibody in which the constant region of an antibody of one species (e.g., rodent, mouse or rat) is replaced with that from a human to achieve a more human-like antibody. Chimeric antibodies may be recombinantly generated by combining the variable light and heavy chain regions obtained from antibody producing cells of one species with the constant light and heavy chain regions from another. In general, chimeric antibodies utilize rodent (or other species, such as rabbit or camelid) variable regions and human constant regions in order to produce an antibody with predominantly human constant domains. The production of chimeric antibodies is well known in the art, and may be achieved by standard means, for example, as described in U.S. Pat. No. 5,624,659, incorporated fully herein by reference.

By “binding to” a molecule is meant having a physicochemical affinity for that molecule or a region of the molecule, e.g., an epitope. Binding may be measured by any of the methods practiced in the art, e.g., using an antibody binding assay or an in vitro translation binding assay.

“Detect” refers to identifying or determining the presence, absence or amount of an analyte to be detected.

By “detectable label” is meant a compound, substance, or composition that, when linked to a molecule of interest, renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition, disorder, or pathology that damages or interferes with the normal function of a cell, tissue, or organ. Examples of diseases include, without limitation, C. difficile infection (CDI), C. difficile-associated diarrhea (CDAD), pseudomembranous colitis (PMC), bowel inflammation, enterocytic detachment, alteration, disruption, or elimination of natural intestinal microflora, and/or paralytic ileus caused by C. difficile infection, or one or more symptoms of these diseases. The production of toxins by the pathogenic and infectious C. difficile microorganisms results in intoxication of the subject (or patient), which is an abnormal state that is a poisoning of the subject (and the subject's cells, tissues and organs) by the presence and activity of the produced toxins.

By “effective amount” is meant the amount of a required to ameliorate, or optimally eliminate, the symptoms of a disease relative to an untreated patient. The effective amount of active compound(s) used to practice the present aspects and embodiments for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

An “epitope tag” refers to a peptide or amino acid sequence (epitope) that is fused, linked, or coupled to a protein, such as a recombinant protein produced by recombinant techniques, and that can be specifically bound by an antibody, e.g., an anti-tag monoclonal antibody or binding molecule that is directed to or generated against the tag peptide or amino acid sequence. Epitope tags are typically short peptide sequences (e.g., from about 5-30 amino acids, or sometimes up to 40 amino acids, that are selected because high-affinity antibodies can be reliably produced in many different species. Such anti-epitope tag antibodies are optimally not cross-reactive with other human peptides or polypeptides and typically do not generate an antibody response, e.g., an anti-tag antibody response, when administered or delivered to a subject. An epitope tag sequence that is fused to a protein provides for the detection and/or purification of the protein using an antibody, e.g., a monoclonal antibody, that specifically binds to the epitope tag. In an embodiment, the protein to which an epitope tag is fused, linked, or coupled is an antibody or VHH protein, e.g., a recombinantly produced antibody or VHH protein. In an embodiment, the VHH is an anti-TcdB VHH antibody. In an embodiment, the protein, or a dimeric or multimeric form thereof, may include one or more epitope tags. In an embodiment, an epitope tag is coupled to the amino (NH) terminus of the protein, e.g., a VHH antibody as described herein In an embodiment, an epitope tag is coupled to the carboxy (COOH) terminus of the protein, e.g., a VHH antibody as described herein. In an embodiment, an epitope tag is coupled to the NH and the COOH termini of the protein, e.g, a VHH antibody as described herein. In an embodiment, a dimeric or multimeric form of the protein includes one or more, e.g., two, three or four, epitope tags linked to one or more of the VHHs comprising the dimeric or multimeric form of the protein. Such epitope tags may be coupled to the VHH components at locations within the dimer or multimer molecule, or at the NH and/or COOH termini of the molecule. In some embodiments, two or more epitope tags may be coupled to a VHH protein in tandem within or at the termini of the VHH protein or dimeric or multimeric form thereof. Examples of epitope tags include, without limitation, FLAG tags (peptide sequence DYKDDDDK (SEQ ID NO: 67) recognized by an anti-FLAG antibody), polyHistidine (His) tags (5-10 histidine residues (SEQ ID NO: 68) (HHHHHH (SEQ ID NO: 69)) bound by a nickel or cobalt chelate), E-tag, a peptide comprising amino acid sequence GAPVPYPDPLEPR (SEQ ID NO: 62) recognized by an antibody; and the epitope tag sequences described herein, which are bound by anti-epitope tag antibodies, forming complexes which may facilitate clearance of the protein containing the tags from the body or system. (See, also, B. Brizzard and R. Chubet. 2001. Curr Protoc Neurosci., Chapter 5. Unit 5.8; DOI: 10.1002/0471142301.ns0508s00; R. Hernan et al., 2000, Biotechniques, 28(4):789-793; C. E. Fritze et al., 2000, Meths Enzymol., 327:3-16; doi: 10.1016/s0076-6879 (00) 27263-7; A. Einhauer et al., 2001, J Biochem Biophys Methods, 49(1-3):455-65, doi: 10.1016/s0165-022x(01)00213-5)).

A “framework (FR) region” or “FR region” includes amino acid residues that are adjacent to the CDRs in V_{H}, and V_{L} regions, and in VHHs. For example, FR region residues may be present in VHHs as described herein, human antibodies, rodent-derived antibodies (e.g., murine and rat antibodies), humanized antibodies, primatized antibodies, chimeric antibodies, antibody fragments (e.g., Fab fragments), VHHs, single-chain antibody fragments (e.g., scFv fragments), antibody domains, and bispecific antibodies, among others. Also by way of example, in a VHH polypeptide sequence, FR1 comprises the amino acids positioned to the left of CDR1; FR2 comprises the amino acids positioned between CDR1 and CDR2; FR3 comprises the amino acids positioned between CDR2 and CDR3; and FR4 comprises the amino acids positioned to the right of CDR3.

By “fragment” is meant a portion of a polypeptide or nucleic acid molecule. This portion contains, preferably, at least 10%, 20%, 30%, 40%, 50%, 60%, 70%, 80%, 90%, or 95% of the entire length of the reference nucleic acid molecule or polypeptide, including percent values between those enumerated. A fragment may contain 10, 20, 30, 40, 50, 60, 70, 80, 90, or 100, 200, 300, 400, 500, 600, 700, 800, 900, or 1000 nucleotides or amino acids. In an embodiment, a fragment or portion possesses or retains activity or function of the polypeptide from which it is derived.

The term “humanized” antibodies refers to forms of non-human (e.g., murine) antibodies, camelid-derived single domain antibody (sdAb) binding molecules, which are comprised of the heavy chain variable (V_{H}) region of heavy-chain-only antibodies (Abs) or VHHs. Humanized antibodies include chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, a humanized antibody or VHH may comprise substantially all of at least one variable domain (or two variable domains in the case of non-VHH antibodies), in which all or substantially all of the CDR regions correspond to those of a non-human immunoglobulin. All or substantially all of the FR regions of a humanized antibody may also be derived from a human immunoglobulin sequence. In the case of non-VHH antibodies, a humanized antibody can also comprise at least a portion of an immunoglobulin constant region (Fc), which may be that of a human immunoglobulin consensus sequence. Techniques and protocols for humanizing antibodies (as well as VHHs) are known and practiced in the art, as described, for examples, in Riechmann et al., Nature. 332:323-7, 1988; Kasmiri et al., Methods, 36(1):25-34, 2005; U.S. Pat. Nos. 5,530,101; 5,585,089; 5,693,761; 5,693,762; and 6,180,370 to Queen et al; EP239400; WO 1991/09967; U.S. Pat. No. 5,225,539; EP592106; and EP519596, the contents of which are incorporated herein by reference. Humanized antibodies or VHHs are molecularly engineered to contain even more human-like immunoglobulin domains, and incorporate only the CDRs of the VHH or animal-derived monoclonal antibody by carefully examining the sequence of the hyper-variable loops of the V regions of the monoclonal antibody or VHH, and fitting them to the structure of the human antibody chains. This process is routinely and commonly carried out by one having skill in the art. See, e.g., U.S. Pat. No. 6,187,287, the contents of which are incorporated by reference herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the aspects and embodiments disclosed and described herein is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the terms “polynucleotide,” “DNA molecule” or “nucleic acid molecule” include both sense and anti-sense strands, cDNA, genomic DNA, recombinant DNA, RNA, mRNA, and wholly or partially synthesized nucleic acid molecules. A nucleotide “variant” is a sequence that differs from the recited nucleotide sequence in having one or more nucleotide deletions, substitutions or additions. Such modifications are readily introduced using standard mutagenesis techniques, such as oligonucleotide-directed site-specific mutagenesis as described, for example, in Adelman et al., 1983, DNA 2:183. Nucleotide variants are naturally-occurring allelic variants, or non-naturally occurring variants. Variant nucleotide sequences in various embodiments exhibit at least about 70%, 75%, 80%, 85%, 90%, 95%, 98%, 99%, or 100% sequence homology or sequence identity to the recited sequence. Such variant nucleotide sequences hybridize to the recited nucleotide sequence under stringent hybridization conditions. In one embodiment, “stringent conditions” refers to prewashing in a solution of 6× SSC, 0.2% SDS; hybridizing at 65° Celsius, 6× SSC, 0.2% SDS overnight; followed by two washes of 30 minutes each in 1× SSC, 0.1% SDS at 65° C., and two washes of 30 minutes each in 0.2× SSC, 0.1% SDS at 65° C.

By “isolated polynucleotide” is meant a nucleic acid (e.g., DNA, cDNA, RNA, mRNA) that is free of the genes, which, in the naturally-occurring genome of the organism from which the nucleic acid molecule of the aspects and embodiments described herein is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, e.g., mRNA, as well as a recombinant DNA that is part of a hybrid gene encoding additional polypeptide sequence.

The terms “protein”, “peptide” and “polypeptide” are used herein to describe any chain of amino acid residues, regardless of length or post-translational modification (for example, glycosylation or phosphorylation). Thus, these terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes full-length proteins, which may be, but need not be, naturally occurring, as well as recombinantly or synthetically produced polypeptides that correspond to a full-length protein, or to particular domains or portions of a protein, which may be, but need not be, naturally occurring. The term also encompasses mature proteins which have an added amino-terminal methionine to facilitate expression in prokaryotic cells. The binding molecules of the aspects and embodiments described herein are encoded by polynucleotides and can be chemically synthesized or synthesized by recombinant DNA methods.

By an “isolated polypeptide” is meant a polypeptide of the aspects and embodiments described herein that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the aspects and embodiments described herein. An isolated polypeptide of the aspects and embodiments described herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, deriving, isolating, or otherwise acquiring the agent.

By “operably linked” is meant the connection between regulatory elements and one or more polynucleotides (genes) or a coding region. That is, gene expression is typically placed under the control of certain regulatory elements, including constitutive or inducible promoters, tissue-specific regulatory elements, and enhancers. A polynucleotide (gene or genes) or coding region is said to be “operably linked to” or “operatively linked to” or “operably associated with” the regulatory elements, meaning that the polynucleotide (gene or genes) or coding region is controlled or influenced by the regulatory elements. The one or more polynucleotides may be separated by spacers or linkers.

By “pathogen” is meant any harmful microorganism, bacterium, virus, fungus, or protozoan capable of interfering with the normal function of a cell. Pathogens as referred to herein produce toxins, e.g., protein toxins, that intoxicate the cells, tissues and organs of a host or recipient organism and cause disease and pathology, often severe, unless they are bound by, neutralized and eliminated from the organism to the extent possible, such as by action of the VHH binding molecules (antibodies) described herein. As described herein, the bacterial pathogen Clostridium difficile produces toxin proteins that intoxicate a subject after infection. Toxin A and toxin B proteins produced by C. difficile translocate to the cytosol of target cells (mainly of the intestinal epithelium, but also in other cells) and inactivate cellular GTP-binding proteins, e.g., Rho, Rac and Cdc42, by monoglucosylation, which causes actin condensation, cell rounding and death. (reviewed by D. Voth and D. Ballard, 2005, Clin. Microbiol. Rev., Vol. 18(2):247-263). In particular, the VHH antibodies described herein are directed against and bind toxin B of C. difficile (TcdB). In embodiments, the anti-TcdB VHHs described herein neutralize TcdB, thereby inhibiting, blocking, or reducing its toxicity and toxic effects in vitro or in vivo.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative or lowering alteration of at least 5%, 10%, 15%, 10%, 25%, 50%, 75%, or 100%.

By “reference” is meant a standard or control condition typically used as a comparator in an assay, test, experiment, or trial, as would be understood by one having skill in the pertinent art. In various nonlimiting embodiments, a reference or control is a different or nonpathogenic protein or cell, such as a non-toxin (or different toxin) protein or a normal cell, a wild-type (unmutated or unaltered) protein, or a healthy subject or individual.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound, molecule, antibody, or VHH that recognizes and binds a protein, peptide, or polypeptide (e.g., an amino acid sequence of the protein, peptide, or polypeptide), but which does not substantially recognize and bind other molecules in a sample, for example, a biological sample, which may contain the protein, peptide, or polypeptide that is specifically bound.

“Nucleic acid” (also called polynucleotide herein) refers to deoxyribonucleotides or ribonucleotides and polymers thereof in either single- or double-stranded form. The term encompasses nucleic acids (polynucleotides) containing known nucleotide analogs or modified backbone residues or linkages, which are synthetic, naturally occurring, and non-naturally occurring, which have similar binding properties as a reference nucleic acid, and which are metabolized in a manner similar to the reference nucleic acid. Examples of such analogs include, without limitation, phosphorothioates, phosphoramidates, methyl phosphonates, chiral methyl phosphonates, 2-O-methyl ribonucleotides, and peptide-nucleic acids (PNAs). Unless otherwise indicated, a particular nucleic acid sequence also implicitly encompasses conservatively modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al., 1991, Nucleic Acid Res, 19:081; Ohtsuka et al., 1985, J. Biol. Chem., 260:2600-2608; Rossolini et al., 1994, Mol. Cell Probes, 8:91-98). The term nucleic acid can be used interchangeably with gene, cDNA, mRNA, oligonucleotide, and polynucleotide.

Nucleic acid molecules or polynucleotides useful in the aspects and embodiments described herein include any nucleic acid molecule or polynucleotide that encodes a polypeptide, e.g., a heteromultimeric binding molecule, of the described aspects and embodiments, or a component or portion thereof. Nucleic acid molecules useful in the methods described herein include any polynucleotide or nucleic acid molecule that encodes a polypeptide e.g., heteromultimeric binding molecule, as described in the aspects and embodiments herein, or a component or portion thereof that has substantial identity to the binding molecule. Such nucleic acid molecules need not be 100% identical with the nucleic acid sequence of the binding molecule, but will typically exhibit substantial identity. Polynucleotides having “substantial identity” to a binding molecule sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, 1987, Methods Enzymol. 152:399; Kimmel, A. R., 1987, Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42°° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42°° C., and even more preferably of at least about 68° C. In a preferred embodiment, wash steps will occur at 25°° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Percentage of sequence identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “homologous” in their various grammatical forms in the context of polynucleotides means that a polynucleotide comprises a sequence that has a desired identity, for example, at least 60% identity, at least 70% sequence identity, at least 80%, at least 85% identity, at least 90% identity; and at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like.

Substantial identity of amino acid sequences, for example, the anti-TcdB polypeptides (C. diff. toxin B-binding VHH polypeptides) refers to sequence identity between or among amino acid sequences of at least 60%, at least 65%, at least 70%, at least 75%, at least 80%, at least 85%, at least 88%, at least 90%, at least 95%, at least 98%, at least 99% or greater sequence identity. In embodiments, 100% identity between or among the amino acid sequences, e.g., the CDR1-3 sequences of the anti-TcdB VHHs as described herein is not required for binding of these polypeptides to toxin B and/or neutralization of toxin. In a particular embodiment, variations between or among VHH amino acid sequences encompass one or more conservative amino acid substitutions in the sequence, for example, as described and presented in Table 3 herein. In an embodiment, one or more conservative amino acid substitutions in an anti-TcdB VHH amino acid sequence may be in one or more CDR sequences, one or more FR sequences, or a combination thereof.

As will be appreciated by the skilled practitioner in the art, some amino acids in a VHH antibody can be modified without significantly altering antigen binding of the VHH antibody. For example, such amino acid sequence modification occurs frequently during in vivo affinity maturation of VHH antibodies, and the best mutations, e.g., for specific and/or high affinity binding to antigen, are positively selected for in the animal during the molecular production of antibodies. It is possible to isolate different VHH intermediates in the affinity maturation process that possess acceptable and specific antigen binding properties and that have significant variations in their CDR sequences.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as, without limitation, a human, a non-human primate, or a bovine, equine, canine, ovine, or feline mammal. Other mammals include rabbits, goats, llamas, mice, rats, guinea pigs, camels and gerbils. In particular, a “subject” as used herein refers to a human subject, such as a human patient or individual. In some cases, the terms subject, patient and individual are used interchangeably herein.

A “VHH binding molecule” or “VHH antibody,” or simply “VHH,” as referred to herein is, in general, a single domain immunoglobulin molecule (antibody) isolated from camelid animals or alpacas, e.g., as described in Maass, D. R., 2007, J. Immunol. Methods, 324(1-2):13-15). A VHH (or VHH antibody) corresponds to the heavy chain of a camelid antibody having a single variable domain (or single variable region), e.g., a camelid-derived single variable H (V_{H}) domain antibody. A VHH has a molecular weight (MW) of about 15 kDa. VHH technology is based on fully functional antibodies from camelids that lack light chains. These heavy-chain antibody molecules contain a single variable domain (VHH) and, typically, two constant domains (CH₂ and CH₃). A cloned (recombinantly produced) and isolated VHH domain is a stable polypeptide harboring the antigen-binding capacity of the original heavy-chain antibody. See, e.g., U.S. Pat. Nos. 5,840,526 and 6,015,695, each of which is incorporated by reference herein in its entirety. VHHs, called NANOBODIES™, may be produced commercially (Ablynx Inc., Ghent, Belgium).

VHHs are efficiently expressed in E. coli, coupled to detection markers, such as a fluorescent marker, or conjugated with enzymes. The small size of VHHs permits their binding to epitopes (antigenic determinants in antigen proteins), e.g., “hidden epitopes” that are not accessible to whole antibodies of much larger size. As a therapeutic, a VHH is capable of efficient penetration and rapid clearance. Its single domain nature allows a VHH to be expressed in a cell without a requirement for supramolecular assembly, as is needed for whole antibodies which are typically tetrameric (two heavy chains and two light chains, having a MW of about 150 kDa). VHHs are also exhibit stability over time and have a longer half-life versus non-VHH antibody molecules, which comprise disulfide bonds that are susceptible to chemical reduction or enzymatic cleavage. Similar to immunoglobulins, VHHs may be modified post-translationally, e.g. to add chemical linkers, detectable moieties, such as fluorescent dyes, enzymes, substrates, chemiluminescent moieties, etc., or specific binding moieties, such as streptavidin, avidin, or biotin, etc., for use in the compositions and methods described herein.

An anti-TcdB VHH polypeptide that specifically binds to and neutralizes the activity of TcdB, may also be referred to as a “VHH-based neutralizing agent (VNA)” a “VNA polypeptide or protein” or a “VNA binding molecule.”

As used herein, the terms “treat,” treating,” “treatment,” and the like refer to reducing, diminishing, abating, alleviating, improving, or ameliorating a disorder and/or symptoms associated therewith. It will be appreciated that, although not precluded, treating a disorder or condition does not require that the disorder, condition or symptoms associated therewith be completely eliminated.

The term “multimeric binding molecule” refers in general to a multi-component protein or polypeptide containing two or more, same or different, VHH binding molecules, which are coupled or linked, e.g., via spacer sequences, to each other and/or other components of the molecule. Multimeric binding molecules may be dimeric, in that the binding molecule contains two VHH polypeptides that bind to toxin B of C. difficile. The anti-TcdB VHH polypeptides in a dimeric multimer may be the same or they may be different VHH polypeptides. The different anti-TcdB VHH polypeptides in a multimeric binding molecule may bind to different regions, portions, or epitopes (e.g., non-overlapping epitopes) of TcdB. Alternatively, the multimeric binding molecules may be heteromultimeric, in that the binding molecule contains more than one, e.g., two, three or four, different anti-TcdB VHH polypeptides such as described herein. In some embodiments, a heteromultimeric binding molecule contains two or more different anti-TcdB VHH polypeptides, each of which specifically binds to toxin B of C. difficile, e.g., at different or non-overlapping epitopes. In embodiments, dimeric multimers and heteromultimeric binding molecules comprising two or more anti-TcdB VHHs bind to and neutralize the activity of TcdB.

As used herein, the terms “prevent,” “preventing,” “prevention,” “prophylactic treatment,” “protection” and the like refer to reducing the probability of developing a disorder or condition in a subject, who does not have, but who is at risk of, is susceptible to, or disposed to (e.g., genetically disposed to), developing a disease, disorder, pathology, or condition.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, inclusive of the first and last values.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Although various features of the present disclosure can be described in the context of a single embodiment, the features can also be provided in separate embodiments, or in any suitable combination or combination of embodiments. The section headings used herein are for organizational purposes only and are not intended to be limiting to the subject matter described.

The features of the present disclosure are set forth with particularity in the appended claims. The features and advantages of the present disclosure will be better understood and obtained by reference to the detailed description infra, which sets forth illustrative embodiments, in which the principles of the disclosure are utilized, and in view of the accompanying drawings as described hereinbelow.

### BRIEF DESCRIPTION OF THE DRAWINGS

FIG. 1 presents a graph showing the results of neutralization studies performed to assess the neutralization activity of representative anti-TcdB VHH antibodies described herein. The assay was performed as described in Examples 4 and 5 infra. In FIG. 1, the anti-TcdB VHHs JZS-C2, JZS-C10, and JZS-E4 displayed IC₅₀ TcdB-neutralization potency near 0.5 nM.

FIG. 2 presents a graph showing the results of neutralization studies performed to assess the neutralization activity of representative anti-TcdB VHH antibodies described herein. The assay was performed as described in Example 4 and 5 infra. In FIG. 2, the anti-TcdB VHH JZS-E6 displayed IC₅₀ TcdB-neutralization potency close to 1 nM; JZS-F6 displayed IC₅₀ TcdB-neutralization potency near 0.5 nM; and JZS-H9 displayed IC₅₀ TcdB-neutralization potency about 15 nM.

FIG. 3 presents a graph showing the results of neutralization studies performed to assess the neutralization activity of representative anti-TcdB VHH antibodies described herein. The assay was performed as described in Example 4 and 5 infra. In FIG. 3, the anti-TcdB VHH JZS-H4 displayed IC₅₀ TcdB-neutralization potency near 0.5 nM; JZT-F7 displayed IC₅₀ TcdB-neutralization potency close to 1 nM; and JZT-G9 displayed IC₅₀ TcdB-neutralization potency about 3 nM.

FIG. 4 presents a graph showing the results of neutralization studies performed to assess the neutralization activity of representative anti-TcdB VHH antibodies described herein. The assay was performed as described in Example 4 and 5 infra. In FIG. 4, the anti-TcdB VHHs JZS-A2a and JZS-B2 displayed minimal TcdB-neutralizing activity, while JZS-B9 displayed IC₅₀ TcdB-neutralization potency close to 1 nM.

FIGS. 5A-5C provide a table in which C. difficile toxin B-binding, anti-TcdB VHH polypeptide sequences of 129 amino acids in length are aligned. In the sequences set forth in FIGS. 5A-5C, linearly from left to right, Framework 1 (FR1) encompasses approximately amino acid residues 5 to 20; complementarity determining region 1 (CDR1) encompasses approximately amino acid residues 21 to 34; Framework 2 (FR2) encompasses approximately amino acid residues 35 to 49/50; complementarity determining region 2 (CDR2) encompasses approximately amino acid residues 49/50 to 59/60; Framework 3 (FR3) encompasses approximately amino acid residues 59/60 to 95/96; complementarity determining region 3 (CDR3) encompasses approximately amino acid residues 95/96 to 117-119; and Framework 4 (FR4) encompasses approximately amino acid residues 117-119 to 129. It will be appreciated that the exact boundaries of the FRs and CDRs are often imprecise, as amino acid sequence variability is typically observed at and near the end of a FR and at and near the start of a hypervariable CDR. Anti-TcdB VHH polypeptide sequences described herein (Example 1) and in Tables 1 and 4, i.e., JZS-C10, JZS-F6, JZS-H4, JZS-B9, JZS-C2, JZS-H9, JZS-E6, JZS-E4, JZS-B2 and JZS-A2a, are representative among the sequences set forth in FIGS. 5A-5C. FIGS. 5A-5C disclose SEQ ID NOs: 123-131, 131-145 and 145-146, respectively, in order of appearance.

FIGS. 6A and 6B illustrate the sequence relatedness of representative anti-TcdB VHH polypeptides in a phylogram or phylogenetic tree (FIG. 6A) and in an amino acid sequence alignment (FIG. 6B). The amino acid sequences of the anti-TcdB VHH polypeptides described in Example I are presented in a phylogram (FIG. 6A), which demonstrates that, comparatively, these VHH polypeptides have approximately 78% to approximately 91% amino acid sequence identity. Despite amino acid sequence variations, each of these VHH polypeptides binds to C. difficile toxin B (TcdB) and in many cases neutralize the toxin activity. The sequence alignments of the anti-TcdB VHH polypeptides described in Example 1 and presented in FIG. 6B shows the areas of the Framework Regions (FR1-FR4) in the sequences. The relatedness of the FRs as described infra can also be visualized in FIG. 6B. FIG. 6B discloses SEQ ID NOs: 7, 15, 17, 5, 19, 13, 11, 3, 9, 23, 120 and 21, respectively, in order of appearance.

FIGS. 7A and 7B present amino acid sequence comparisons of camelid VHH polypeptides that specifically bind to C. difficile toxin B (TcdB). FIG. 7A shows the amino acid sequences and designates the CDR1, CDR2 and CDR3 regions of 15 individual anti-TcdB VHH polypeptides (109 amino acids in length) of a family of anti-TcdB VHHs generated as described herein. FIG. 7B shows the amino acid sequences and designates the CDR1, CDR2 and CDR3 regions of 5 of the most diverse members of the anti-TcdB VHH family. FIG. 7B illustrates that CDR diversity is selected for during the process of affinity maturation of a TcdB antigen binding VHH in the B cells of the immunized animal, yet all of the anti-TcdB VHH polypeptides that were generated specifically and detectably bound to TcdB. In the sequences of this anti-TcdB VHH polypeptide family, the FR regions (FR1, FR2, FR3 and FR4) are essentially invariant between and among the individual sequences, e.g., 97% or greater sequence identity. FIGS. 7A and 7B disclose 129, 124, 129, 129, 121, 147-150, 147, 151-153, 147, 154, 121, 148, 155, 124 and 153, respectively, in order of appearance.

### DETAILED DESCRIPTION OF THE DISCLOSURE

Described and featured herein are single domain antibody (sdAb) binding molecules. which are comprised of the heavy chain variable (V_{H}) region of heavy-chain-only antibodies (Abs), that specifically bind to the toxin B (TcdB) exotoxin virulence factor produced by pathogenic Clostridium difficile (C. diff.) microorganisms. Single-domain antibodies (camelid single-domain antibodies) are called VHHs as they derive from the V_{H} region of a class of heavy-chain-only antibodies. The anti-toxin B (TcdB) VHHs were produced from immunized camelids (alpacas) and were selected for their ability to specifically bind to TcdB and, in most cases, to neutralize TcdB and the critical pathogenic functions caused by this toxin. The anti-TcdB VHHs as described herein are C. diff toxin B binding poly peptides comprising hypervariable variable regions (CDRs) within framework (FR) regions. In general, the FRs of the anti-TcdB VHHs are typically highly similar in amino acid sequence, or differ by conservative amino acid substitutions at certain positions of the FR sequences. among different anti-TcdB VHHs or families of anti-TcdB VHHs. The anti-TcdB VHHs as described herein are stable over time and provide therapeutic efficacy in animal models. The anti-TcdB VHHs are employed as therapeutic antitoxin agents for the prevention and treatment of toxin B-mediated diseases, such as CDI and its symptoms, caused by Clostridium diffiicile. It will be understood that the terms “anti-TcdB VHH antibody.” “anti-TcdB VHH polypeptide.” “anti-TcdB VHH antibody polypeptide,” and anti-TcdB VHH″ are used interchangeably herein.

The presence of toxins produced by C. diffiicile, in particular, toxin B (TcdB), in the general circulation and locally in the gastrointestinal (GI) tract, causes serious illness in infected humans and other animals. C. difficile TcdB is considered to be the key exotoxin that causes CDI pathology. Antitoxins are therapeutic agents that prevent toxin infection or reduce further development of negative and adverse symptoms in patients who have been exposed to the toxin (a process referred to as “intoxication”). Antitoxins have historically included antisera obtained from large animals (e.g., sheep, horse, and pig) that were immunized with inactivated or non-functional toxin. More recently, antitoxin therapies have been developed using combinations of antitoxin monoclonal antibodies, including yeast-displayed single-chain variable fragment antibodies generated from vaccinated humans or mice. See, e.g., Nowakowski et al. 2002. Proc Natl Acad Sci USA, 99: 11346-11350; Mukherjee et al. 2002. Infect Immun, 70: 612-619; Mohamed et al. 2005 Infect Immun, 73: 795-802; Walker, K. 2010 Interscience Conference on Antimicrobial Agents and Chemotherapy—50th Annual Meeting—Research on Promising New Agents: Part 1. IDrugs 13: 743-745. Drawbacks to the production and use of antisera and monoclonal antibodies as antitoxins include their difficulty to produce economically at scale. The production of such products typically requires long development times and frequently results in problematic quality control, shelf-life and safety issues.

In general, antitoxins function through two key mechanisms, namely, neutralization of toxin function and clearance of the toxin from the body. Toxin neutralization occurs through biochemical processes including inhibition of enzymatic activity and prevention of binding to cellular receptors. Antibody mediated serum clearance occurs subsequent to the binding of multiple antibodies to the target antigen (Daeron M. 1997 Annu Rev Immunol, 15: 203-234; Davies et al. 2002 Arthritis Rheum, 46: 1028-1038; Johansson et al. 1996 Hepatology. 24: 169-175; and Lovdal et al. 2000 J Cell Sci, 113 (Pt 18): 3255-3266).

The anti-TcdB VHHs or multimeric forms thereof as described herein are provided as beneficial therapeutic agents and/or antitoxins that bind to C. difficile toxin B. In some cases, the anti-TcdB VHHs or multimeric forms thereof both promote toxin B neutralization by rapidly and effectively blocking further toxin B activity and also accelerate clearance of toxin B from the system to eliminate future pathology. In addition, increased stability and longevity of the anti-TcdB VHHs or multimeric forms thereof in the GI tract where they can bind to and neutralize the effects of toxin B contribute to the advantages of these molecules as antitoxins that provide greater therapeutic efficacy as a treatment for CDI and other diseases and symptoms caused by C. diff. infection and intoxication.

In some embodiments, the binding activity and/or neutralizing activity of the anti-TcdB VHHs described herein, or multimeric forms thereof, in the absence of any epitope tag sequences are significantly effective such that the antitoxin function of these molecules obviates the need for an anti-tag antibody or clearing antibody.

VHHs, such as the anti-TcdB VHHs described herein, have a number of advantages over conventional antibodies and recombinant antibody domains, including (i) they are small monomeric proteins (14 kDa) that express and fold efficiently in recombinant hosts; (ii) they are more stable to extremes of pH and temperature compared with conventional antibodies; (iii) they typically bind conformational epitopes, and thus are more likely to neutralize target functions; and (iv) they are amenable to designed multimerization which often leads to higher potencies and a reduction in the risk that microorganisms (e.g., C. diff) will develop resistance; and (v) they offer more therapeutic versatility, such as multispecificity, thus supporting their beneficial utility in treating enteric diseases.

The amino acid sequences of representative anti-TcdB VHH antibodies described herein are set forth in SEQ ID NOs: 1, 3, 5, 7, 9, 11, 13, 15, 17, 19, 21 and 23, and the corresponding polynucleotide sequences encoding each of the representative anti-TcdB VHH antibodies are set forth in SEQ ID NOs: 2, 4, 6, 8, 10, 12, 14, 16, 18, 20, 22 and 24 (Example 1). The binding regions of the anti-TcdB VHHs include CDRs (CDR1, CDR2 and CDR3) as set forth in the sequences in Example 1 and in Table 1 below. The CDR binding regions are positioned within framework (FR) regions of the VHH polypeptide, which do not vary substantially in sequence between discrete anti-TcdB VHHs and which provide a “structural scaffold” for the CDRs, which bind to TcdB. By way of non-limiting example, the binding of CDRs within FRs to a target protein (antigen) may be via conformational binding or interaction, electrostatic binding interaction, hydrogen bonding, Van der Waals forces, or hydrophobic bonding, or combinations thereof, as would be appreciated by those having skill in the art.

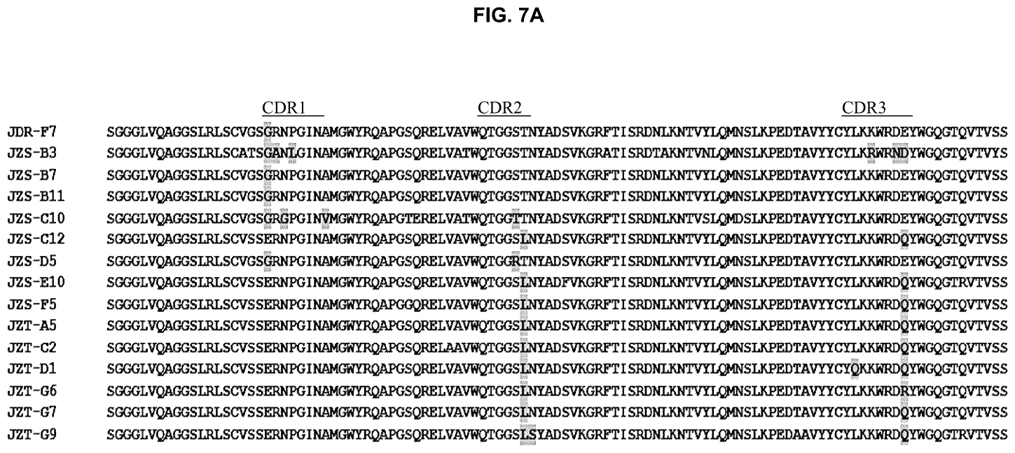

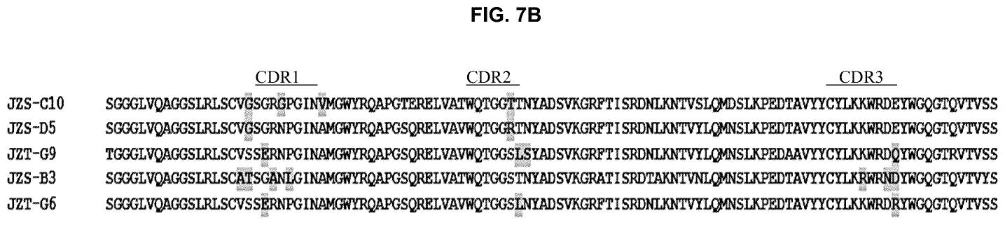

The CDRs of the anti-TcdB VHH polypeptides described herein may vary in amino acid sequence length. By way of nonlimiting example, CDR1 of the anti-TcdB VHH polypeptides as described herein may comprise from about 6 to about 12 amino acid residues; CDR2 may comprise from about 7 to about 12 amino acid residues; and CDR3 may comprise from about 8 to about 21 amino acid residues. It will be appreciated by one skilled in the art that number of amino acids that constitute a CDR is not necessarily precise. In some cases, an amino acid residue, or 2 or 3 amino acid residues, at one end or both ends of a given CDR may be considered as part of the CDR or as part of the neighboring FR region. The CDR regions of representative anti-TcdB VHH antibody polypeptides generated from camelid alpacas as described herein (Example 1) are presented in Table 1 below. In addition, FIG. 7A presents the amino acid sequences of a family of anti-TcdB VHH antibody polypeptides (JZS-C10 family) generated from a camelid alpaca immunized with TcdB as described herein. FIG. 7B presents the amino acid sequences of the most diverse members of anti-TcdB VHH antibody polypeptides in the JZS-C10 family, all of which bind to C. difficile toxin B (TcdB). The anti-TcdB VHH antibodies in FIG. 7B demonstrate the CDR diversity that is selected during affinity maturation of TcdB binding polypeptides in the same animal. Despite such CDR diversity, the TcdB binding VHHs generated as described herein show detectable binding to TcdB. As observed from the sequence alignments shown in FIGS. 7A and 7B, in the context of the four VHH framework regions, which do not vary significantly in sequence among different anti-TcdB VHH polypeptides, the CDR1 sequences of the anti-TcdB VHH polypeptide members of the JZS-C10 family may vary by about 42% to about 58%; the CDR2 sequences of the anti-TcdB VHH polypeptide members of the JZS-C10 family may vary by about 27%; and the CDR3 sequences of the anti-TcdB VHH polypeptide members of the JZS-C10 family may vary by about 30% to about 40%. Notwithstanding some variation among the CDR sequences in the context of their framework regions, the anti-TcdB VHH polypeptides still bind very well to the TcdB antigen.

TABLE 1

| VHH | | | |
|---|---|---|---|
| (anti- | | | |
| TcdB VHH) | CDR1 | CDR2 | CDR3 |
| JZS-A2a | GSVYTF | SGGTITK | NAGDTIAQAMGTRRFPFDR |
| (XAF-1) | (SEQ ID NO: 25) | (SEQ ID NO: 26) | (SEQ ID NO: 27) |
| JZS-B2 | GTSFPRNY | SHDGNVE | KLVTLRRDEY |
| (XAF-4) | (SEQ ID NO: 28) | (SEQ ID NO: 29) | (SEQ ID NO: 30) |
| JZS-B9 | RFSLINYA | TSGGATY | AAGPYSRTLVSRWKVGDGM |
| (XAF-5) | (SEQ ID NO: 31) | (SEQ ID NO: 32) | EY (SEQ ID NO: 33) |
| JZS-C2 | GFTSNSYY | SSSGGSPN | AASKFPLTTMASNRYHY |
| (XAF-6) | (SEQ ID NO: 34) | (SEQ ID NO: 35) | (SEQ ID NO: 36) |
| JZS-C10 | GRGPGINV | QTGGTTN | YLKKWRDEY |
| (XAF-9) | (SEQ ID NO: 37) | (SEQ ID NO: 38) | (SEQ ID NO: 39) |
| JZS-E4 | GSSFSMNV | RSDGITN | FHGRARTGNNADLGS |
| (XAF-10) | (SEQ ID NO: 40) | (SEQ ID NO: 41) | (SEQ ID NO: 25) |
| JZS-E6 | GRLSERIF | SRLGRAN | NLKPFVDNYR |
| (XAF-11) | MIST (SEQ ID NO: 43) | (SEQ ID NO: 44) | (SEQ ID NO: 25) |
| JZS-F6 | GITFSNVA | STGGSSTS | VKGPKYSATIRRPE |
| (XAF-12) | (SEQ ID NO: 46) | (SEQ ID NO: 47) | (SEQ ID NO: 25) |
| JZS-H4 | GFNFSVQI | STGGASKS | SKGPRTWINSSPR |
| (XAF-13) | (SEQ ID NO: 49) | (SEQ ID NO: 50) | (SEQ ID NO: 25) |
| JZS-H9 | GTAFSLDT | SSSGASN | YRGRVRGVWPLDSGMMY |
| (XAF-14) | (SEQ ID NO: 52) | (SEQ ID NO: 53) | (SEQ ID NO: 25) |
| JZT-F7 | GSILSS | SRTGATD | NAGLGMGDPRRPGPW |
| (XAG-1) | (SEQ ID NO: 55) | (SEQ ID NO: 56) | (SEQ ID NO: 57) |
| JZT-G9 | ERNPGINA | QTGGSLS | YLKKWRDQY |
| (XAG-7) | (SEQ ID NO: 58) | (SEQ ID NO: 70) | (SEQ ID NO: 60) |

In view of the representative anti-TcdB VHH amino acid sequences shown in FIGS. 7A and 7B, it will be appreciated by one skilled in the art that individual VHH polypeptides, (e.g., of about 109 amino acids in length and comprising 3 CDRs and 4 FR regions), which comprise at least about or equal to 85%, or 88%, or greater identity in amino acid sequence (e.g., about 12-15% variation in amino acid sequence) bind to TcdB antigen. In addition, the TcdB binding VHHs may further neutralize TcdB toxin. In an embodiment, at least about or equal to 85%, 86%, 87%, 88%, 89%, 90%, 91%, 92%, 93%, 94%, 95%, 96%, 97%, 98%, 99%, or 100% amino acid sequence identity is tolerated among the anti-TcdB VHHs without adversely affecting or eliminating binding of the VHH polypeptides to the TcdB antigen. In an embodiment, such amino acid sequence variation among the anti-TcdB VHH polypeptides is tolerated in the CDRs of the VHH polypeptides without adversely affecting binding of the VHHs to TcdB. In a particular embodiment, the amino acid sequence variations between or among anti-TcdB VHHs encompass one or more conservative amino acid substitutions or changes in a VHH amino acid sequence. In an embodiment, the one or more conservative amino acid substitutions or changes in a VHH amino acid sequence occur in one or more CDR sequences of the VHH, in one or more FR sequences of the VHH, or in CDR and FR sequences of the VHH.

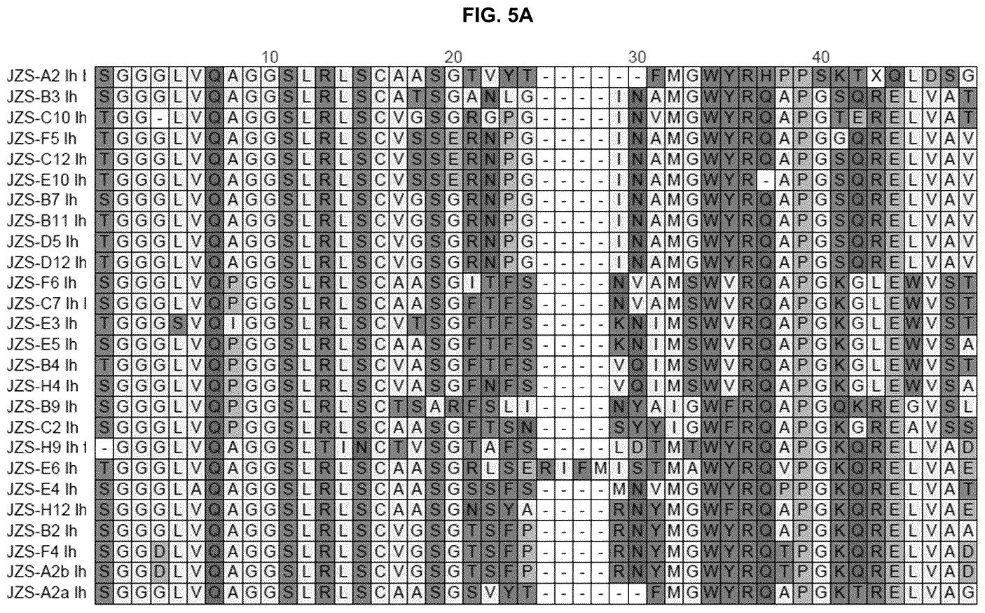

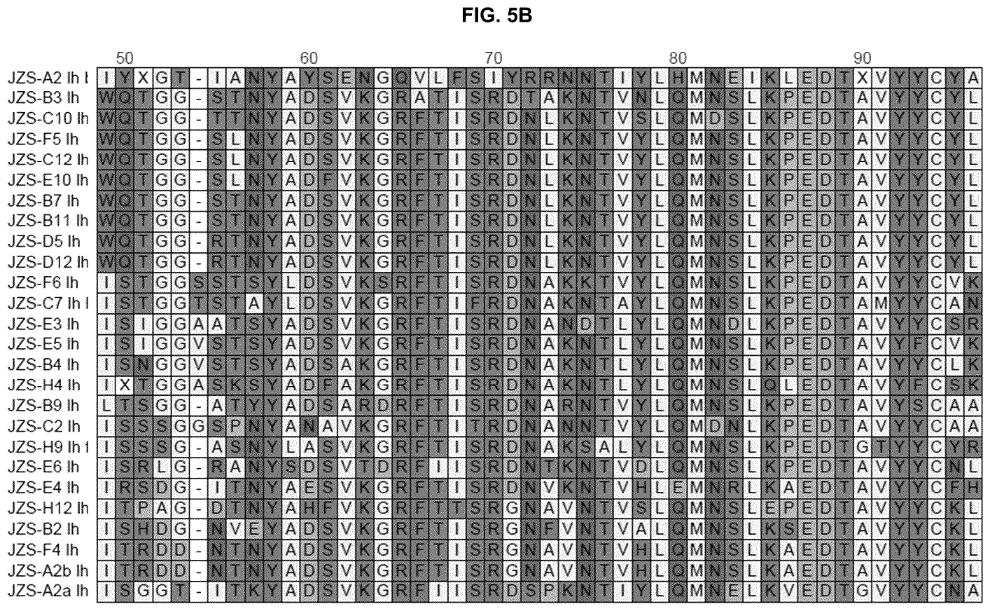

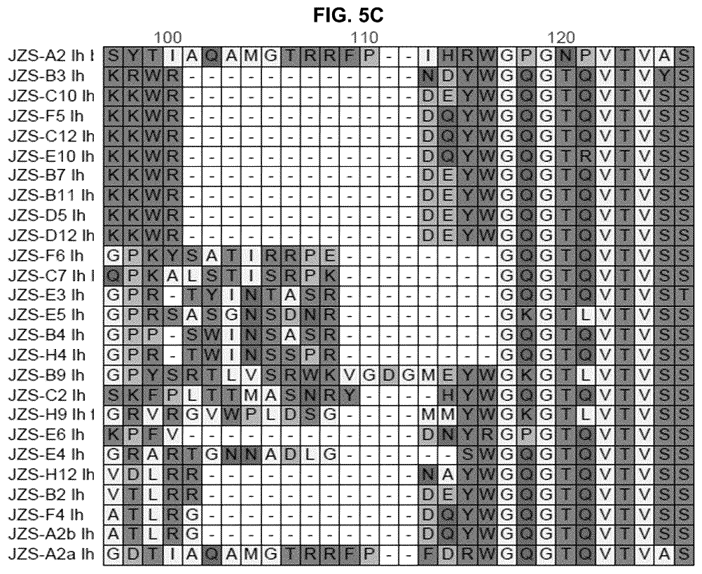

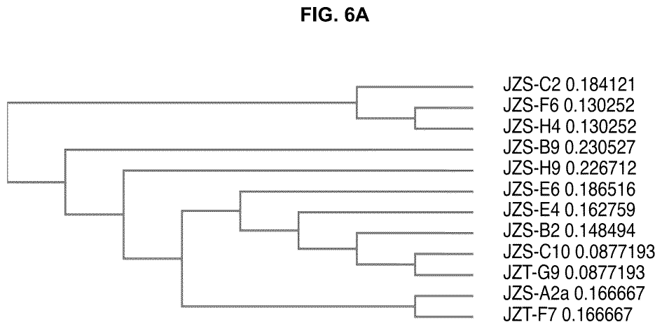

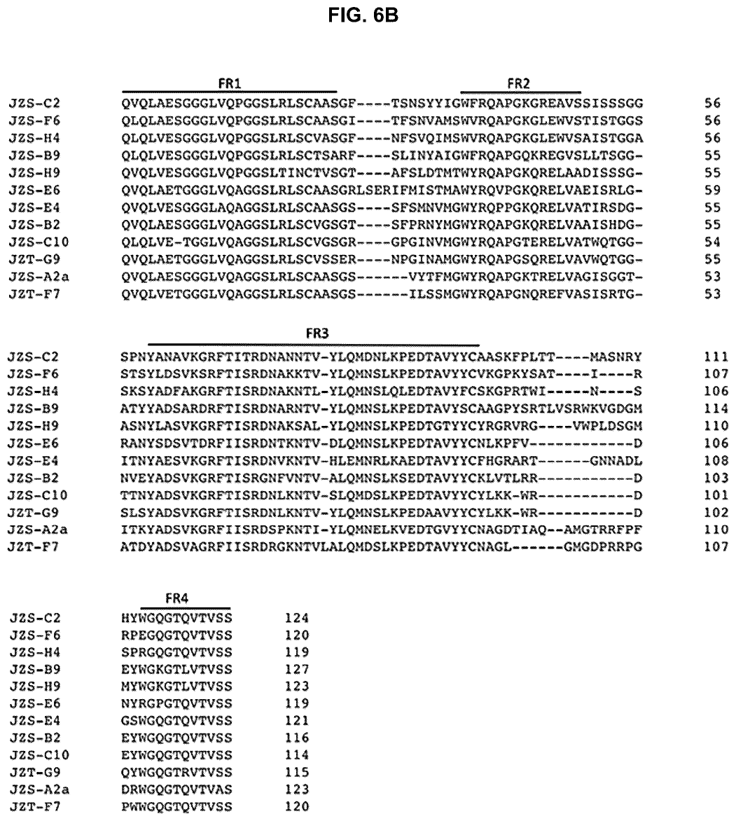

The three CDRs of the anti-TcdB VHH polypeptides are arranged or positioned in the context of four FR regions as follows: FR1-CDR1-FR2-CDR2-FR3-CDR3-FR4, in which FR1 to FR4 refer to the framework regions 1-4, respectively, and in which CDR1 to CDR3 refer to the complementarity determining regions 1-3, respectively. An alignment of anti-TcdB VHHs, all of which specifically bind to toxin B, demonstrates the extensive similarities among the sequences of each of the FRs (FR1, FR2, FR3 and FR4) found in the different toxin B binding VHH polypeptides (FIGS. 5A-5C; FIG. 6B; FIGS. 7A and 7B). Similar to the FRs in conventional antibody polypeptides, the respective FR regions (FR1, FR2, FR3 and FR4) of the anti-TcdB VHH polypeptides described herein are highly similar in sequence not only among different TcdB-binding VHHs but also among camelid VHH polypeptides that bind to other antigens. By way of example and as shown in Table 2, anti-TcdB VHH FR regions are highly similar in sequence to the respective FR1, FR2, FR3 and FR4 sequences of an unrelated camelid VHH polypeptide (see, L. S. Mitchell and L. J. Colwell, 2018, Proteins. 86(7): 697-706; A. M. Vattekatte et al., March 2020, PeerJ., 6(8):e8408. DOI: 10.7717/peerj.8408), thus evidencing that the FR regions FR1, FR2, FR3 and FR4 of different VHHs do not vary significantly in sequence. In Table 2, the FRs of the VHH in the publication of Mitchell and Colwell is used as a reference sequence. Accordingly, provided are anti-TcdB VHH polypeptides comprising CDR1-3, in the structural context of FR1-4, that bind to and/or neutralize toxin B protein of C. diffiicile, or to suitable fragments of toxin B, as well as polypeptides that comprise or consist essentially of one or more of the anti-TcdB VHHs and/or toxin B binding fragments thereof.

Table 2 presents the amino acid sequences of the four framework regions, i.e., FR1, FR2, FR3 and FR4, respectively, of ten representative anti-TcdB VHH polypeptides described herein (i.e., JZS-C10, JZS-F6, JZS-H4, JZS-B9, JZS-C2, JZS-H9, JZS-E6, JZS-E4, JZS-B2 and JZS-A2a), relative to the FR1-FR4 of a VHH reference as reported in L. S. Mitchell and L. J. Colwell, 2018, Proteins. 86(7): 697-706, termed “Reference sequence” herein), thus demonstrating the substantial similarities among the structural FRs of camelid VHHs, independent of antigen binding specificity. In Table 2, the amino acid (AA) position numbering of the camelid VHH sequences containing the FRs begins at residue “Q” as #1, as shown in the amino acid sequences of the anti-TcdB VHH polypeptides presented in Example 1. It will be appreciated that in the sequence alignments presented in FIGS. 5A-5C, the amino acid residues #1 and #2, etc., shown in the figure, i.e., “E” and “S/T, etc.,” correspond to amino acid positions 6 and 7, etc., respectively, of the sequences presented in Example 1.

TABLE 2
FR1

| | | Position # | | | | | | | | | | | | | | |
|---|---|---|---|---|---|---|---|---|---|---|---|---|---|---|---|---|
| | | 1 | 2 | 3 | 4 | 5 | 6 | 7 | 8 | 9 | 10 | 11 | 12 | 13 | 14 | 15 |
| Reference | AA | Q | V | Q | L | Q/V | E | S | G | G | G | L/S | V | Q | A/P | G |
| Sequence | | | | | | | | | | | | | | | | |
| Anti- | AA | | | | | | E | S/T | G | G | G | L | V | Q | A/P | G |
| TcdBVHHs | | | | | | | | | | | | | | | | |

FR1

| | | Position # | | | | | | | | | |
|---|---|---|---|---|---|---|---|---|---|---|---|
| | | 16 | 17 | 18 | 19 | 20 | 21 | 22 | 23 | 24 | 25 |
| Reference | AA | G | S | L | R | L | S | C | A | A | S |
| Sequence | | | | | | | | | | | |
| Anti- | AA | G | S | L | R/T | L/I | S/N | C | A/V/T | A/G/V/S | S/A |
| TcdBVHHs | | | | | | | | | | | |

FR2

| | | Position # | | | | | | | | | | | | | |
|---|---|---|---|---|---|---|---|---|---|---|---|---|---|---|---|
| | | 36 | 37 | 38 | 39 | 40 | 41 | 42 | 43 | 44 | 45 | 46 | 47 | 48 | 49 |
| Reference | AA | W | F/Y/V | R | Q | A | P | G | K | E/C/G | R/L | E | F/G/L/W | V | A/S/T |
| Sequence | | | | | | | | | | | | | | | |
| Anti- | AA | W | F/Y/V | R | Q | A/P | P | G | K/S/Q | Q/G/E/K/T | R/L | E | L/W/G | V/D | A/S |
| TcdBVHHs | | | | | | | | | | | | | | | |

FR3

| | | Position # | | | | | | | | | |
|---|---|---|---|---|---|---|---|---|---|---|---|
| | | 60 | 61 | 62 | 63 | 64 | 65 | 66 | 67 | 68 | 69 |
| Reference | AA | Y | A/Q/T/V | D/E | S | V/A | K | G | R | F | T/A |
| Sequence | | | | | | | | | | | |
| Anti- | AA | Y | A/L/S | D/N/A/E | S/F/A | V/A | K/R/T | G/S/D | R | F | T/I |
| TcdBVHHs | | | | | | | | | | | |

FR3

| | | Position # | | | | | | | | |
|---|---|---|---|---|---|---|---|---|---|---|
| | | 70 | 71 | 72 | 73 | 74 | 75 | 76 | 77 | 78 |
| Reference | AA | I/V | S | R/Q | D | N/K | A | K/A | N | T |
| Sequence | | | | | | | | | | |
| Anti- | AA | I | S/T | R | D/G | N/S | A/L/T/V/F/P | K/N/V | N/K//S | T/A |
| TcdBVHHs | | | | | | | | | | |

FR3

| | | Position # | | | | | | | | | | | | | | | | | |
|---|---|---|---|---|---|---|---|---|---|---|---|---|---|---|---|---|---|---|---|
| | | 79 | 80 | 81 | 82 | 83 | 84 | 85 | 86 | 87 | 88 | 89 | 90 | 91 | 92 | 93 | 94 | 95 | 96 |
| Reference | AA | V/L/M | Y | L | Q | M | N/D | S/N | L | K/R | P | E/D | D | T | A/G | V/I/T/M | Y | Y | C |
| Sequence | | | | | | | | | | | | | | | | | | | |
| Anti- | AA | V/L | Y/H/D/S | L | Q/E | M | N/D | S/N/R/E | L | K/Q | P/L/S/V | E | D | T | A/G | V/T | Y | Y/F/S | C |
| TcdBVHHs | | | | | | | | | | | | | | | | | | | |

FR4

| | | Position # | | | | | | | | | | |
|---|---|---|---|---|---|---|---|---|---|---|---|---|
| | | 117 | 118 | 119 | 120 | 121 | 122 | 123 | 124 | 125 | 126 | 127 |
| Reference | AA | W | G | Q | G | T | Q | V | T | V | S | S |
| Sequence | | | | | | | | | | | | |
| Anti- | AA | W/R | G | Q/K/P | G | T | Q/L | V | T | V | S/A | S |
| TcdBVHHVHHs | | | | | | | | | | | | |

(Table 2 discloses SEQ ID NOs: 114-119, 79 and 122, respectively, in order of appearance)

In embodiments, in cases in which a FR (or CDR) amino acid residue in a VHH polypeptide may be one of several alternative amino acid residues, the alternative amino acid residues will frequently share similar characteristics or properties, e.g., hydrophobicity, polarity, and/or charge. A conservative replacement (also called a conservative substitution) is an amino acid replacement or substitution in a polypeptide or region thereof that changes a given amino acid residue to a different amino acid residue with similar biochemical properties, such as charge, hydrophobicity, and/or size. By way of non-limiting example, the below Table 3 presents amino acids and their 1-letter codes categorized into six main classes based on their structure and the general chemical characteristics of their side chains (R groups).

TABLE 3

| Amino Acids | Class |
|---|---|
| Glycine (G), Alanine (A), Valine (V), | Aliphatic |
| Leucine (L), Isoleucine (I) | |
| Serine (S), Cysteine (C), | Hydroxyl |
| Selenocysteine (U), Threonine (T), | or sulfur/ |
| Methionine (M) | selenium |
| | containing |
| Proline (P) | Cyclic |
| Phenylalanine (F), Tyrosine (Y), | Aromatic |
| Tryptophan (W) | |
| Histidine (H), Lysine (K), Arginine (R) | Basic |
| Aspartate (D), Glutamate (E), | Acidic and |
| Asparagine (N), Glutamine (Q) | amides |
| | thereof |

In an embodiment, amino acid sequence substitutions or changes in an anti-TcdB VHH polypeptide relative to another anti-TcdB VHH polypeptide comprise conservative amino acid substitutions or changes such that a given amino acid residue is substituted with or replaced by a different amino acid residue with similar biochemical properties, such as charge, hydrophobicity, and/or size. In an embodiment, sequence variation between or among anti-TcdB VHH polypeptides results from one or more conservative amino acid changes and account for the percent sequence variation, e.g., 85%, 86%, 87%, 88%, 90%, 91%, 92%, 93%, 94%, 95%, 96%, 97%, 98%, or 99% sequence variation.

In some embodiments, the VHHs as described herein are humanized using methods and techniques practiced by those having skill in the art. (See, e.g., U.S. Pat. Nos. 8,975,382 and 10,550,174, the contents of which are incorporated by reference herein).

The anti-TcdB VHH antibodies described herein have widespread application as therapeutics in the treatment of disease and pathologies resulting from infection by C. difficile pathogenic bacteria and the toxins, particularly, toxin B produced by these pathogens. The described anti-TcdB VHHs described herein are particularly useful for binding to and eradicating the disease-causing the TcdB target protein that intoxicates a subject after C. difficile infection and causes debilitating disease, and even death. In embodiments, various embodiments encompass polynucleotides (nucleic acid sequences) that encode the operably linked modular components that constitute the described anti-TcdB VHHs. In embodiments, the anti-TcdB VHHs are recombinantly produced. In embodiments, the anti-TcdB VHHs encompass the proteins (polypeptides) encoded by the polynucleotides. In embodiments, the polynucleotide is DNA, cDNA, RNA, mRNA, and the like. In an embodiment, the anti-TcdB VHHs may be humanized or codon-optimized using methods practiced by those having skill in the art.

### Polynucleotides Encoding VHHs That Bind to C. difficile Toxin B (TcdB)

In some cases, more than one anti-TcdB-binding VHH antibody (i.e., anti-TcdB VHH) is coupled or linked (e.g., covalently linked) to other sequences, e.g., a leader amino acid sequence, one or more spacer (flexible spacer) amino acid sequences, or one or more epitope tag amino acid sequences, to produce a multimeric VHH binding molecule containing two or more, e.g., three, four, five, or six, VHHs linked together. In an embodiment, a polynucleotide molecule, such as a recombinant or isolated polynucleotide molecule, encodes a single anti-TcdB VHH or more than one anti-TcdB VHH linked together to form a multimer (i.e., a multimeric anti-TcdB VHH binding molecule). In an embodiment, the polynucleotide encodes a fragment or portion of the anti-TcdB VHH or multimeric anti-TcdB VHH binding molecule, in particular, a fragment or portion that maintains TcdB binding function or TcdB binding and neutralizing function. The polynucleotide sequences encoding representative anti-TcdB VHH antibodies as described herein are set forth in SEQ ID NOs: 2, 4, 6, 8, 10, 12, 14, 16, 18, 20, 22 and 24 (Example 1).

In an embodiment, an anti-TcdB VHH can be humanized, i.e., modified to increase its similarity to antibodies or antibody variants produced naturally in humans, using techniques known and practiced in the art. Briefly and by way of nonlimiting example, a humanized antibody can be generated by inserting the appropriate CDR coding sequences (e.g., ‘donor’ sequences that are responsible for the desired binding properties) into a human antibody “scaffold” (e.g., ‘acceptor’ sequences) comprising essentially invariant framework region (FR) sequences (FRs). In embodiments, the CDRs of the anti-TcdB VHH antibodies described herein may be inserted into FRs, which provide the structural scaffold that allows the CDRs to bind to, and in certain cases, to neutralize, toxin B. Recombinant DNA methods using an appropriate vector and expression in mammalian cells are employed and routinely practiced in the art to achieve the production of recombinant humanized antibodies.

In an embodiment, the polynucleotide encodes a TcdB binding and neutralizing function, or a functional binding portion thereof, that includes an epitope tag. In embodiments, antibody fragments, microproteins, darpins, anticalins, peptide mimetic molecules, aptamers, synthetic molecules, etc. can be linked to the multimeric anti-TcdB VHH binding molecule. In embodiments, a multimeric anti-TcdB VHH binding molecule may contain two of the same anti-TcdB VHHs, e.g., a dimeric form, or two different anti-TcdB VHHs described herein. In other embodiments, a multimeric anti-TcdB VHH binding molecule may contain more than two anti-TcdB VHHs in combination, e.g., a combination of three, four, or five, etc. anti-TcdB VHHs linked together. In an embodiment, the anti-TcdB VHH components of a multimeric anti-TcdB VHH binding molecule may be linked covalently.

In an embodiment, an anti-TcdB VHH can be modified, for example, by attachment (e.g., directly or indirectly via a linker or spacer) to another anti-TcdB VHH. In some embodiments, anti-TcdB VHH is attached or genetically (recombinantly) fused to another anti-TcdB VHH. Accordingly, a polynucleotide (e.g., DNA) that encodes one anti-TcdB VHH is joined (in reading frame) with the polynucleotide encoding a second anti-TcdB VHH, and so on. In certain embodiments, additional amino acids are encoded within the polynucleotide between the anti-TcdB VHHs so as to produce an unstructured region (e.g., a flexible spacer) that separates the anti-TcdB VHHs, e.g., to better promote independent folding of each anti-TcdB VHH antibody into its active or functional conformation or shape. Commercially available techniques for fusing proteins (or their encoding polynucleotides) may be employed to recombinantly join or couple the anti-TcdB VHHs into multimeric anti-TcdB VHHs containing two or more of the same or different anti-TcdB VHHs as described herein.

Polynucleotide sequences encoding the anti-TcdB VHHs or multimeric forms thereof as described herein can be recombinantly expressed and the resulting encoded anti-TcdB VHH antibody molecules can be produced at high levels and isolated and/or purified. In an embodiment, the recombinant anti-TcdB VHHs or multimeric forms thereof are produced in soluble form. In an embodiment, a recombinantly produced anti-TcdB VHH is dimeric, such that two anti-TcdB VHHs, same or different, are joined or linked together. In an embodiment, a recombinantly produced anti-TcdB VHH is multimeric, e.g., a tetramer, which contains four anti-TcdB VHH antibodies, the same or a combination of different anti-TcdB VHHs, joined together. By way of example, a tetramer may contain four of the same anti-TcdB VHHs joined together, or a combination of four different anti-TcdB VHHs, or two pairs of the same anti-TcdB VHHs, joined together. In an embodiment, the anti-TcdB VHH or multimeric forms thereof are contained in pharmaceutically acceptable compositions for use in treating CDI and/or neutralizing toxin B produced by C. diff.

The compositions and methods described herein in various embodiments include an isolated polynucleotide sequence or an isolated polynucleotide molecule that encodes an anti-TcdB VHH or multimeric form thereof. Accordingly, in some embodiments, the isolated polynucleotide sequence or isolated polynucleotide molecule comprises or consists of a polynucleotide sequence that encodes a polypeptide molecule (anti-TcdB VHH) having an amino acid sequence of SEQ ID NOS: 1, 3, 5, 7, 9, 11, 13, 15, 17, 19, 21, 23, or a functional portion thereof, as described herein. In some embodiments, the isolated polynucleotide sequence or isolated polynucleotide molecule comprises or consists of a polynucleotide sequence of SEQ ID NOS: 2, 4, 6, 8, 10, 12, 14, 16, 18, 20, 22, or 24. In an embodiment, a composition comprises a combination of the isolated polynucleotide sequences or isolated polynucleotide molecules as described herein.

Also encompassed by the aspects and embodiments described herein are polynucleotide sequences, DNA or RNA, which are substantially complementary to the DNA sequences encoding the polypeptides described herein, and which specifically hybridize with these DNA sequences under conditions of stringency known to those of skill in the art. As referred to herein, substantially complementary means that the nucleotide sequence of the polynucleotide need not reflect the exact sequence of the original encoding sequences, but must be sufficiently similar in sequence to permit hybridization with a nucleic acid sequence under high stringency conditions. For example, non-complementary bases can be interspersed in a nucleotide sequence, or the sequences can be longer or shorter than the polynucleotide sequence, provided that the sequence has a sufficient number of bases complementary to the sequence to allow hybridization thereto. Conditions for stringency are described, e.g., in Ausubel, F. M., et al., Current Protocols in Molecular Biology, (Current Protocol, 1994), and Brown, et al., Nature. 366:575 (1993); and further defined in conjunction with certain assays.

Vectors, plasmids, bacteria, viruses, or genetically modified Spirulina algal organisms containing one or more of the polynucleotide molecules encoding the anti-TcdB VHH amino acid sequence of SEQ ID NO: 1, 3, 5, 7, 9, 11, 13, 15, 17, 19, 21, 23, or a functional portion thereof, are provided. Vectors, plasmids, bacteria, viruses, or genetically modified Spirulina algal organisms containing one or more of the polynucleotide molecules of SEQ ID NOS: 2, 4, 6, 8, 10, 12, 14, 16, 18, 20, 22, or 24 are also provided. Suitable vectors for use in eukaryotic and prokaryotic cells are known in the art and are commercially available or readily prepared by the skilled practitioner in the art. Additional vectors can also be found, for example, in Ausubel, F. M., et al., Ibid. and in Sambrook et al., “Molecular Cloning: A Laboratory Manual,” 2nd ED. (1989), and other editions.

Any of a variety of expression vectors (prokaryotic or eukaryotic) known to and used by those of ordinary skill in the art may be employed to express recombinant polypeptides described herein. Expression can be achieved in any appropriate host cell that has been transformed or transfected with an expression vector containing a polynucleotide (DNA) molecule that encodes a recombinant polypeptide. Suitable host cells include prokaryotes, yeast and higher eukaryotic cells. By way of example, the host cells employed include, without limitation, E. coli, yeast, insect cells, or a mammalian cell line such as COS or CHO. The DNA sequences expressed in this manner can encode any of the polypeptides described herein, including variants thereof.

Uses of plasmids, vectors or viruses containing polynucleotides encoding the anti-TcdB VHHs or multimeric forms thereof as described herein include generation of mRNA or protein in vitro or in vivo. In related embodiments, host cells transformed with the plasmids, vectors, or viruses are provided, as described above. Nucleic acid molecules can be inserted into a construct (such as a prokaryotic expression plasmid, a eukaryotic expression vector, or a viral vector construct, which can, optionally, replicate and/or integrate into a recombinant host cell by known methods. The host cell can be a eukaryote or prokaryote and can include, for example and without limitation, yeast (such as Pichia pastoris or Saccharomyces cerevisiae), bacteria (such as E. coli, or Bacillus subtilis), animal cells or tissue (CHO or COS cells), insect Sf9 cells (such as baculoviruses infected SF9 cells), or mammalian cells (somatic or embryonic cells, Human Embryonic Kidney (HEK) cells, Chinese hamster ovary (CHO) cells, HeLa cells, human 293 cells and monkey COS-7 cells). Suitable host cells also include a mammalian cell, a bacterial cell, a yeast cell, an insect cell, a plant cell, or a Spirulina algal cell.

An anti-TcdB VHH-encoding polynucleotide molecule can be incorporated or inserted into the host cell by known methods. Examples of suitable methods for transfecting or transforming host cells include, without limitation, calcium phosphate precipitation, electroporation, microinjection, infection, lipofection and direct uptake. “Transformation” or “transfection” as appreciated by the skilled practitioner refers to the acquisition of new or altered genetic features by the incorporation of additional nucleic acids, e.g., DNA, into cellular DNA. “Expression” of the genetic information of a host cell is a term of art which refers to the directed transcription of DNA to generate RNA that is, in turn, translated into a polypeptide (anti-TcdB VHH antibody). Procedures for preparing recombinant host cells and incorporating nucleic acids are described in more detail in Sambrook et al., “Molecular Cloning: A Laboratory Manual,” Second Edition (1989) and Ausubel, et al. “Current Protocols in Molecular Biology,” (1992), and later editions, for example.

A transfected or transformed host cell is maintained under suitable conditions for expression and recovery of the polypeptides described herein. In certain embodiments, the cells are maintained in a suitable buffer and/or growth medium or nutrient source for growth of the cells and expression (and secretion) of the gene product(s) into the growth medium. The type of growth medium is not critical to the aspects and embodiments herein and is generally known to those skilled in the art, such as, for example, growth medium and nutrient sources that include sources of carbon, nitrogen and sulfur. Examples include Luria-Bertani (LB) broth, Superbroth, Dulbecco's Modified Eagles Media (DMEM), RPMI-1640, M199 and Grace's insect media. The growth medium can contain a buffering agent, as commonly used in the art. The pH of the buffered growth medium may be selected and is generally a pH that is tolerated by, or optimal for, growth of the host cell, which is maintained under a suitable temperature and atmosphere.

In another aspect, an RNA polynucleotide, in particular, mRNA, encodes the anti-TcdB VHHs or multimeric forms thereof as described herein. mRNA encoding the anti-TcdB VHHs or multimeric forms thereof may contain a 5′ cap structure, a 5′ UTR, an open reading frame, a 3′ UTR and poly-A sequence followed by a C30 stretch and a histone stem loop sequence (Thess, A. et al., 2015, Mol Ther, 23(9):1456-1464; Thran, M. et al., 2017, EMBO Molecular Medicine, DOI: 10.15252/emmm.201707678). Sequences may be codon-optimized for human use using techniques and protocols known and used by those skilled in the art. In an embodiment, the mRNA sequences do not include chemically modified bases. mRNAs encoding the anti-TcdB VHHs or multimeric forms thereof as described herein may be capped enzymatically or further polyadenylated for in vivo studies/use.

Expression of proteins, which normally have a shortened serum half-life, by encoding mRNA, particularly sequence optimized, unmodified mRNA, advantageously prolongs the bioavailability of these proteins for in vivo activity. (see, e.g., K. Kariko et al, 2012, Mol. Ther., 20:948-953; Thess, A. et al., 2015, Mol Ther, 23(9):1456-1464;). Accordingly, anti-TcdB VHHs or multimeric forms thereof with an estimated serum half-life of 1-2 days are likely to benefit from being encoded by mRNA. Of note, the half-lives of neutralizing VHH protein serum titers at one to three days after treatment were estimated to be, on average, 1.5-fold higher than from day three onward, even without target-specific mRNA optimization. (Mukherjee et al., 2014, PLoS ONE, 9e 106422). In general, one to three days after treatment, both mRNA and protein half-lives contribute to the kinetics of serum titers, while after day three forward, the kinetics is almost exclusively determined by the properties of the expressed protein.

### Multimeric Forms of the Anti-TcdB VHHs

Multimeric forms of the anti-TcdB VHH antibodies described herein are encompassed by the present disclosure. Such multimeric anti-TcdB VHHs contain more than one anti-TcdB VHH antibody that binds to TcdB. In an embodiment, a multimer of anti-TcdB VHH antibodies contains two anti-TcdB VHHs, same or different, that bind to TcdB. Such a multimeric form of the anti-TcdB VHH molecules constitutes a dimeric multimer. In an embodiment, the dimeric multimer comprises two of the same anti-TcdB VHH antibodies coupled using a flexible linker. In an embodiment, the dimeric multimer comprises two, different anti-TcdB VHH antibodies coupled using a flexible linker. In an embodiment, the two, different anti-TcdB VHH antibodies bind to different, nonoverlapping epitopes of TcdB.

In another embodiment, a multimer of anti-TcdB VHH antibodies contains more than two (e.g., three, four, five, six, etc.) anti-TcdB VHHs, same or different, that bind to TcdB. Such a multimeric form of the anti-TcdB VHH molecules may comprise three or more of the same anti-TcdB VHH antibodies coupled using flexible linkers. In an embodiment, the anti-TcdB VHH multimer comprises a combination or mixture of the anti-TcdB VHH antibodies described herein coupled using a flexible linker. In some cases, the multimeric form of the anti-TcdB VHH antibodies may contain more than one of the same anti-TcdB VHH antibody and/or different, or different combinations of, anti-TcdB VHH antibodies coupled using flexible linker or spacer peptides. Nonlimiting examples of flexible linking amino acid sequences include amino acid sequence GGGGS (SEQ ID NO: 63); GGGGSGGGGSGGGGS (SEQ ID NO: 64), or a functional portion thereof; EPKTPKPQGGGGSGGGGSGGGGSQGVQSQVQLVE (SEQ ID NO: 65); or EPKTPKPQ (SEQ ID NO: 66). In certain embodiments, the anti-TcdB VHH amino acid sequences described herein are coupled to epitope tag amino acid sequences as described infra, or to other sequences. In another embodiment, a dimerization agent that complexes peptide fragments each containing at least about 5 to 25 amino acids, 25 to 50 amino acids, 50 to 100 amino acids, 100 to 150 amino acids, and 150 amino acids to about 200 amino acids may be used. Multimerization agents and methods of using the agents for forming multimeric binding proteins can be found, for example, in U.S. Pat. Nos. 9,023,352, 8,349,326 and 7,763,445, each of which is incorporated by reference herein in its entirety.

In embodiments, the multimeric forms of the anti-TcdB VHH antibodies described herein both bind to TcdB and neutralize its activity.

### Epitope Tags and Antibodies Thereto

In certain embodiments, an anti-TcdB VHH antibody, or a dimeric or multimeric form thereof, includes a single epitope tag (single tag sequence) or multiple tags (multiple tag sequences), to which anti-tag antibodies specifically bind. For example, a multimeric VHH may include at least one, or two or more, epitope tags in the molecule. Such epitope tags, which are specifically bindable by the anti-epitope tag antibodies, are useful in detecting VHHs bound to toxin antigens. In addition, such tags may facilitate clearance of VHHs bound to toxin antigens following binding of the tags by anti-tag antibody. By way of nonlimiting example, a tag may constitute an O-tag epitope of amino acid sequence DELGPRLMGK (SEQ ID NO: 61) or an E-tag epitope of amino acid sequence GAPVPYPDPLEPR (SEQ ID NO: 62). The epitope tags may be placed at the amino terminus, carboxy terminus, or internally within a multimeric VHH molecule. Such tags and/or anti-tag antibodies are described for example, in (U.S. Pat. Nos. 8,349,326; 9,023,352, WO 2019/094095A1) and U.S. Pat. Nos. 7,943,345; 8,114,634 and 8,865,871), the contents of which are incorporated herein by reference in their entireties. An example of an anti-O tag monoclonal antibody (IgG1) suitable for binding the DELGPRLMGK (SEQ ID NO: 61) tag sequence is described in WO 2019/094095A1, the contents of which are fully incorporated by reference. By way of illustrative example, peroxidase labeled antibodies that bind the anti-O-tag antibody may be used to detect these anti-tag antibodies in assays in which samples are incubated with goat anti-O-tag-HRP conjugated antibody (Bethyl labs) diluted 1:5000 in blocking solution for 1 hour at RT with rocking and were washed as above before adding TMB microwell peroxidase substrate (KPL) to develop (incubated for 10-40 min). Development was stopped with 1M H₂SO₄ and the plates were read at 450 nm on an ELx808 Ultra Microplate Reader (Bio-Tek instruments), (Mukherjee, J. et al., 2012, PloS ONE 7:e29941).

In some cases, an albumin binding peptide (DICLPRWGCLWED (SEQ ID NO: 71)), may be included at the 3′ end of an anti-TcdB VHH antibody or multimeric form thereof.

In certain embodiments, the presence of an epitope tag operably linked, coupled, or fused to a VHH antibody or multimeric form thereof, wherein the tag is bound by an anti-epitope tag antibody, induces clearance of the toxin-bound VHH molecule from the body. In an embodiment, the binding of one or more epitope tags in an anti-TcdB VHH molecule by anti-epitope tag antibody(ies) may synergistically induce clearance of TcdB from the body following binding by the VHH or multimeric form thereof.

In an aspect, an anti-tag (i.e., anti-epitope tag) antibody may be administered to a subject who is also treated with or administered an anti-TcdB VHH or multimeric form thereof containing one or more epitope tags, or a pharmaceutical composition thereof. The anti-tag antibodies bind to the epitope tags of the anti-TcdB VHH, which, in turn, binds to one or more toxin B proteins, thereby forming a complex that is rapidly cleared from the body (Sepulveda, J. et al., 2010, Infect. Immun., 78(2):756-763; Mukherjee, J. et al., 2012, PLoS ONE. 7(1): e29941. PMCID: PMC3253120; https://doi.org/10.1371/journal.pone.0029941). In an embodiment, an anti-epitope tag monoclonal antibody of a specific isotype, for example IgG1, or a binding fragment or portion thereof that binds to the tag sequence, or a molecule containing its CDR components that bind to the tag sequence, may be provided to a subject who is also administered one or more anti-TcdB VHHs or a multimeric form thereof as described herein. The administration or co-administration of an anti-tag antibody advantageously enhances clearance from the body of a complex formed by toxin TcdB bound by anti-TcdB VHH or a multimeric form thereof, which is, in turn, bound by an anti-epitope tag antibody or binding portion thereof.

In certain embodiments, an anti-tag antibody may also effect or facilitate immunoglobulin effector functions. Anti-tag antibodies may include, for example, IgA, IgD, IgE, IgG, and IgM immunoglobulins and subtypes thereof. An immune response to an epitope tag included in an anti-TcdB VHH or multimeric form thereof may involve the elicitation of specific monoclonal antibodies and/or polyclonal antibodies that specifically bind to the tag. Immunoglobulin effector functions may involve, for example, interaction(s) between the Fc portion of the immunoglobulin and receptors or other protein molecules in a subject or cells thereof. Depending on the immunoglobulin type, the effector functions result in clearance of the disease agent (e.g., excretion, degradation, lysis or phagocytosis). In an embodiment, an anti-tag antibody of one immunoglobulin effector type binds to an anti-TcdB VHH or multimeric form thereof which comprises one or more epitope tags. In embodiments in which a multimeric form of an anti-TcdB VHH comprises at least one epitope tag, or two or more epitope tags, an anti-tag antibody, or binding portion thereof, binds to each of the tags of the multimeric molecule. In embodiments, the epitope tags may be the same or different in a given anti-TcdB VHH multimeric molecule. Without wishing to be bound by theory, the presence of more than one epitope tag bindable by an anti-epitope tag antibody, or binding portion thereof, in a multimeric form of an anti-TcdB VHH may increase the rate and/or level of clearance of toxin B bound to the anti-TcdB VHH multimer in a subject.

Suitable methods of producing or isolating antibody fragments having the requisite binding specificity and affinity for binding to an epitope tag include for example, methods which select recombinant antibody from a library or by PCR (e.g., U.S. Pat. Nos. 5,455,030 and 7,745,587 each of which is incorporated by reference herein in its entirety).

Functional fragments of antibodies, including fragments of chimeric, humanized, primatized, veneered, or single chain antibodies, can also be produced. Functional fragments or portions of the foregoing antibodies include those which are reactive with the toxin protein. For example, antibody fragments capable of binding to the toxin protein or a portion thereof, include, but not limited to, scFvs, Fabs, VHHs, Fv, Fab, Fab′ and F(ab′)₂. Such fragments can be produced by enzymatic cleavage or by recombinant techniques. For instance, papain or pepsin cleavage are used generate Fab or F(ab′)₂ antibody fragments, respectively. Antibody fragments are produced in a variety of truncated forms using antibody-encoding genes in which one or more stop codons has been introduced upstream of the natural stop site. For example, a chimeric gene encoding a F(ab′)₂ heavy chain peptide portion can be designed to include DNA sequences encoding the CHI peptide domain and hinge region of an immunoglobulin heavy chain.

### Pharmaceutical Compositions

Also featured herein are methods for treating or preventing pathologies and disease caused by toxin B produced by C. difficile following infection of a subject by C. difficile microorganisms. The methods include administering to a subject in need thereof an amount of an anti-TcdB VHH or multimeric anti-TcdB VHH binding molecule that is effective to specifically bind to and optimally neutralize C. difficile toxin B. In an embodiment, if an anti-TcdB VHH or multimeric anti-TcdB VHH binding molecule includes an epitope tag, an anti-epitope tag antibody may be administered to the subject. (see, e.g., WO 2019/094095A1, the contents of which is incorporated by reference herein in its entirety). In an embodiment, an anti-TcdB VHH or multimeric anti-TcdB VHH binding molecule is provided or used in a pharmaceutical composition.

Typically, a carrier or excipient is included in a composition as described herein, such as a pharmaceutically acceptable carrier or excipient, which includes, for example, sterile water, aqueous saline solution, aqueous buffered saline solutions, aqueous sucrose, dextrose, or mannose solutions, aqueous glycerol solutions, ethanol, calcium carbonate, albumin, starch, cellulose, silica gel, polyethylene glycol (PEG), dried skim milk, rice flour, magnesium stearate, and the like, or combinations thereof. The terms “pharmaceutically acceptable carrier” and a “carrier” refer to any generally acceptable excipient or drug delivery device that is relatively inert and non-toxic.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's The Science and Practice of Pharmacy Ed. by LWW 21^{st} EQ. PA, 2005 discloses various carriers used in formulating pharmaceutical compositions and known techniques for the preparation thereof. Carriers are selected to prolong dwell time for example following any route of administration, including IP, IV, subcutaneous, mucosal, sublingual, inhalation or other form of intranasal administration, or other route of administration.

Some examples of materials that can serve as pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The preparation of such compositions and solutions ensuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, intravenous, oral, and the like.

In certain embodiments, these compositions optionally further comprise one or more additional therapeutic agents. In certain embodiments, the additional therapeutic agent(s) is/are selected from antibiotics particularly antibacterial compounds, anti-viral compounds, anti-fungals. In some embodiment, additional therapeutic agent(s) may include one or more of growth factors, anti-inflammatory agents, vasopressor agents, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGF), epidermal growth factor (EGF), platelet derived growth factor (PDGF), neu differentiation factor (NDF), hepatocyte growth factor (HGF), and hyaluronic acid.

In an aspect, according to the methods of treatment described herein, immunization is promoted by contacting the subject with a pharmaceutical composition containing an anti-TcdB VHH or multimeric form thereof, as described herein. Thus, methods are provided for immunization, comprising administering to a subject in need thereof, such as a subject infected with C. diff. or a subject having CDI or symptoms thereof, a therapeutically effective amount of a pharmaceutical composition comprising an anti-TcdB VHH or multimeric form thereof as active agent for a time necessary to achieve the desired result. It will be appreciated that the methods encompass protectively administering a composition comprising an anti-TcdB VHH or multimeric form thereof as a preventive or therapeutic measure to ameliorate, reduce, abrogate, or diminish infection or the effects thereof by C. diffiicile, thus, minimizing complications associated with a slow development of immunity or response to infection (especially in compromised patients such as those who are nutritionally challenged, or at risk patients such as the elderly or infants).

A therapeutically effective dose refers to that amount of active agent which ameliorates at least one symptom or condition. Therapeutic efficacy and toxicity of active agents can be determined by standard pharmaceutical procedures in cell cultures or experimental animals, e.g., ED₅₀ (the dose that is therapeutically effective in 50% of the population) and LD₅₀ (the dose that is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices are especially useful. The data obtained from cell culture assays and from animal studies are used in formulating a range of dosages for human administration. By way of example, a therapeutic dose may be at least about 1 μg per kg, at least about 5, 10, 50, 100, 500 μg per kg, at least about 1 mg/kg. 5, 10, 50 or 100 mg/kg body weight of a composition or active component thereof per body weight of the subject, although the doses may be more or less depending on age, health status, history of prior infection, and immune status of the subject as would be known by one of skill in the art. Doses may be divided or unitary and may be administered once daily, or repeated at appropriate intervals.

### Administration of Pharmaceutical Compositions

After formulation with an appropriate pharmaceutically acceptable carrier in a desired dosage, a pharmaceutical composition comprising an anti-TcdB VHH or multimeric form thereof, or an anti-TcdB VHH or multimeric form thereof, can be administered to humans and other mammals by routes known and practiced in the art.

The administration of an anti-TcdB VHH or multimeric form thereof, or a pharmaceutical composition comprising of an anti-TcdB VHH or multimeric form thereof, as a therapeutic for the treatment or prevention of disease or pathology caused by toxin B produced by C. difficile following infection may be by any suitable means that results in a concentration of the therapeutic that, combined with other components, if desired, is effective in ameliorating, reducing, eliminating, abating, or stabilizing disease, pathology, or the symptoms thereof in a subject. The therapeutic may be administered systemically, for example, formulated in a pharmaceutically-acceptable composition or buffer such as physiological saline.

Routes of administration include, for example and without limitation, subcutaneous, intravenous, intraperitoneal, intramuscular, intrathecal, intraperitoneal, or intradermal injections that provide continuous, sustained levels of the therapeutic in the subject. Other routes include, without limitation, gastrointestinal, esophageal, oral, rectal, intravaginal, etc. The amount of the therapeutic to be administered varies depending upon the manner of administration, the age and body weight of the subject, and with the clinical symptoms of the bacterial infection or associated disease, pathology, or symptoms. Generally, amounts will be in the range of those used for other agents used in the treatment of disease or pathology associated with C. difficile infection, although in certain instances, lower amounts may be suitable because of the increased range of protection and treatment afforded by the described anti-TcdB VHHs or multimeric forms thereof as therapeutics. A composition is administered at a dosage that ameliorates, decreases, diminishes, abates, alleviates, or eliminates the effects of the bacterial (microorganism) infection or disease (e.g., CID or the symptoms thereof) as determined by a method known to one skilled in the art.

In embodiments, a therapeutic or prophylactic treatment agent may be contained in any appropriate amount in any suitable carrier substance, and is generally present in an amount of 1-95% by weight of the total weight of the composition. The composition may be provided in a dosage form that is suitable for parenteral (e.g., subcutaneous, intravenous, intramuscular, intrathecal, or intraperitoneal) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions may in some cases be formulated to release the active agent substantially immediately upon administration or at any predetermined time or time period after administration. The latter types of compositions are generally known as controlled release formulations, which include (i) formulations that create a substantially constant concentration of a therapeutic agent or drug within the body over an extended period of time; (ii) formulations that after a predetermined lag time create a substantially constant concentration of a therapeutic agent or drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the gut or gastrointestinal system; (v) formulations that allow for convenient dosing, such that doses are administered, for example, once every one or two weeks; and (vi) formulations that target a disease using carriers or chemical derivatives to deliver the therapeutic agent or drug to a particular cell type. For some applications, controlled release formulations obviate the need for frequent dosing during the day to sustain a therapeutic level in plasma, serum, or blood. In an embodiment, one or more anti-TcdB VHHs or multimeric forms thereof may be formulated with one or more additional components for administration to a subject in need, e.g., patients who have contracted C. difficile infection and suffer from the serious repercussions of toxin production by these microorganisms.

Any of a number of strategies can be pursued in order to obtain controlled release of a therapeutic agent in which the rate of release outweighs the rate of metabolism of the therapeutic agent or drug in question. In one example, controlled release is obtained by appropriate selection of various formulation parameters and ingredients, including, e.g., various types of controlled release compositions and coatings. Thus, the therapeutic agent or drug may be formulated with appropriate excipients into a pharmaceutical composition that, upon administration, releases the therapeutic agent or drug in a controlled manner. Examples include single or multiple unit tablet or capsule compositions, oil solutions, suspensions, emulsions, microcapsules, microspheres, molecular complexes, nanoparticles, patches, and liposomes.

Compositions for parenteral or oral use may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent (i.e., an anti-TcdB VHH or multimeric form thereof) that reduces or ameliorates a disease, pathology, or symptom thereof, such as CID, C. difficile-associated diarrhea (CDAD), pseudomembranous colitis (PMC), bowel inflammation, enterocytic detachment, alteration, disruption, or elimination of natural intestinal microflora, and/or paralytic ileus caused by C. difficile infection, the composition may include suitable parenterally acceptable carriers and/or excipients. In some cases, an active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, compositions comprising an anti-TcdB VHH or multimeric form thereof are sterilized and, if desired, mixed with auxiliary agents, e.g., lubricants, preservatives, stabilizers, wetting agents, emulsifiers, salts for influencing osmotic pressure, buffers, coloring, and/or aromatic substances and the like which do not deleteriously react with the active compounds. In some embodiments, an anti-TcdB VHH or multimeric form thereof are combined, where desired, with other active substances, e.g., enzyme inhibitors, to reduce metabolic degradation. An effective amount of a pharmaceutical composition can vary according to choice or type of anti-TcdB VHH or multimeric form thereof as described herein, the particular composition formulated, the mode of administration and the age, weight and physical health or overall condition of the patient, for example. In an embodiment, an effective amount of an anti-TcdB VHH or multimeric form thereof and/or anti-epitope tag antibody is an amount which is capable of reducing one or more symptoms of disease or pathology caused by C. diff. infection and the production of its toxins, namely, toxin B.

In certain embodiments, a composition includes one or more polynucleotide sequences that encode one or more anti-TcdB VHHs or multimeric forms thereof as described herein. In an embodiment, a polynucleotide sequence encoding an anti-TcdB VHH or multimeric form thereof is in the form of a DNA molecule or multimer. In some embodiments, the composition includes a plurality of nucleotide sequences each encoding an anti-TcdB VHH or multimeric form thereof, or any combination of anti-TcdB VHHs described herein, such that the anti-TcdB VHH antibodies or multimers thereof are expressed and produced in situ. In such compositions, a polynucleotide sequence is administered using any of a variety of delivery systems known to those of ordinary skill in the art, including eukaryotic, bacterial, viral vector nucleic acid expression systems, or Spirulina-based delivery as described infra. Suitable nucleic acid expression systems contain appropriate nucleotide sequences operably linked for expression in a patient (such as suitable promoter and termination signals). Bacterial delivery systems involve administration of a bacterium that secretes or expresses the polypeptide on its cell surface, e.g., probiotic E. coli as described infra. In an embodiment, a polynucleotide molecule encoding an anti-TcdB VHH or multimeric form thereof can be introduced using a viral expression system (e.g., vaccinia or other pox virus, retrovirus, lentivirus, or adenovirus associated virus (AAV)), which uses a non-pathogenic (defective), replication competent virus. Techniques for incorporating nucleic acid (DNA) into such expression systems are well known to and practiced by those of ordinary skill in the art. The nucleic acid (DNA) can also be “naked,” as described, for example, in Ulmer et al., 1993, Science, 259:1745-1749 and as reviewed by Cohen, 1993, Science 259:1691-1692. The uptake of naked DNA can be increased by the use of nanoparticles comprising DNA or coating the DNA onto biodegradable beads, which are efficiently transported into recipient cells.

### Therapeutic Methods

Methods of treating disease, conditions, pathology and/or symptoms thereof associated with C. difficile infection are provided. The methods comprise administering a therapeutically effective amount of an anti-TcdB VHH or multimeric form thereof as described herein, or a pharmaceutical composition comprising these agents to a subject (e.g., a mammal such as a human). In an embodiment, the method is for treating a subject suffering from or susceptible to CDI, C. difficile-associated diarrhea (CDAD), pseudomembranous colitis (PMC), bowel inflammation, enterocytic detachment, alteration, disruption, or elimination of natural intestinal microflora, and/or paralytic ileus caused by C. difficile infection, or a symptom thereof. The method includes administering to the subject a therapeutically effective amount of an anti-TcdB VHH, multimeric form thereof, or composition thereof sufficient to treat the disease, illness, condition, disorder and/or symptom thereof, under conditions such that the disease or disorder and/or symptom thereof is treated.

The therapeutic methods include prophylactic as well as therapeutic treatment. In an embodiment, the treatment method includes administering a therapeutically effective amount of an anti-TcdB VHH or multimeric form thereof as described herein, or a pharmaceutical composition comprising these agents, before or during the time that a subject is administered one or more antibiotics, or a treatment involving a course of antibiotics, to treat a different bacterial disease or infection. As will be appreciated by the skilled practitioner in the art, antibiotic treatment can disturb, disrupt, or otherwise adversely affect the normal microflora, microfauna and microbiome in the GI tract of a subject, thereby allowing C. difficile bacteria to proliferate, or over-proliferate, causing CDI and other C. difficile promoted disease or disorders and symptoms thereof. Accordingly, providing a subject with an anti-TcdB VHH or multimeric form of the anti-TcdB VHHs provides a beneficially useful and practical prophylactic and/or therapeutic treatment regimen for a subject in need.

A subject or patient includes an animal, particularly a mammal, and more particularly, a human. Such an anti-TcdB VHH or multimeric form thereof as described herein, or a pharmaceutical composition comprising these agents, used as therapeutics in treatments will be suitably administered to subjects or patients suffering from, having, susceptible to, or at risk for a disease, disorder, or symptom thereof caused by or associated with infection by C. difficile microorganisms and toxin B produced by C. diff. Determination of patients who are “susceptible” or “at risk” can be made by any objective or subjective determination obtained by the use of a diagnostic test or based upon the opinion of a patient or a health care provider (e.g., genetic test, enzyme or protein marker, family history, and the like). Identifying a subject in need of such treatment can be in the judgment of a subject himself or herself, or of a health care/medical professional and can be subjective (e.g., opinion) or objective (e.g., measurable or quantifiable by a test or diagnostic method).

### Methods of Delivery

In an embodiment, an anti-TcdB VHH or multimeric form thereof as described herein, or a pharmaceutical composition comprising these agents, can be administered to a subject in need of treatment for intoxication by a toxin B protein (TcdB). In an embodiment, a mixture of anti-TcdB VHHs or multimeric forms thereof can be administered to a subject in need of treatment for infection or intoxication by C. diff. In an embodiment, the anti-TcdB VHH or multimeric form thereof may include one or more epitope tag sequences to which anti-epitope tag antibody(ies) specifically bind. In another embodiment in which the anti-TcdB VHH or multimeric form thereof administered to a subject includes one or more epitope tag sequences, a specific anti-epitope tag antibody can also be administered to the subject.

In some embodiments, the administration of two or more anti-TcdB VHHs or multimeric forms thereof may increase the effectiveness of the therapy to treat infection by C. diff. and TcdB production and reduce the severity of one or more negative symptoms related to exposure of the subject to TcdB. In an embodiment, administering to a subject the anti-TcdB VHH or multimeric form thereof that includes one or more, e.g., two, epitope tag sequences may result in improved therapy, treatment, or protection against disease caused by C. diff. and its production of TcdB. In an embodiment, the epitope tag sites of the anti-TcdB VHH or multimeric form thereof are bound by a specific anti-tag antibody. The administration of an anti-TcdB VHH or multimeric form thereof as described herein, or a composition comprising the agent, and the administration of one or more anti-epitope tag antibodies may be performed simultaneously or sequentially in time. In an embodiment, an anti-TcdB VHH or multimeric form thereof is administered before, after, or at the same time as the administration of another anti-TcdB VHH or multimeric form thereof, or before administration of an anti-tag antibody, provided that the anti-TcdB VHH(s) or multimeric form(s) thereof and/or the anti-tag antibody(ies) are administered close enough in time to have the desired effect (e.g., before the anti-TcdB VHHs or multimeric forms thereof have been cleared by the body). Accordingly, “co-administration” embraces the administration of an anti-TcdB VHH or multimeric form thereof and a subsequent anti-TcdB VHH or multimeric form thereof, or the anti-tag antibody, at time points that will achieve effective treatment of disease caused by C. diff. toxin B, such as reduction in the level of toxin B and disease and symptoms associated with the presence of the toxin. The described methods are not limited by time intervals between which an anti-TcdB VHH or multimeric form thereof and/or the anti-tag antibody(ies) are administered; provided that these agents, or compositions containing these agents, are administered close enough in time to produce or achieve the desired effect. In an embodiment, only an anti-TcdB VHH or multimeric form thereof is administered to a subject in need thereof. In another embodiment, an anti-TcdB VHH or multimeric form thereof and an anti-epitope tag antibody are premixed and administered together, or are not premixed but are co-administered within minutes of each other. In other embodiments, the anti-TcdB VHH or multimeric form thereof and anti-epitope tag antibody(ies) are co-administered with other medications, drugs, compounds, or compositions suitable for treating the disease agent.

In yet other embodiments, an anti-TcdB VHH or multimeric form thereof, or a composition containing the agent(s), is administered to a subject prior to the potential risk of exposure to infection by toxin B-producing C. difficile in order to protect a subject from disease and symptoms caused by toxin B. For example, an anti-TcdB VHH or multimeric form thereof and/or anti-epitope tag antibody (“clearing antibody”) is administered minutes, hours or days prior to the risk of exposure to infection by C. diffiicile. Alternatively, an anti-TcdB VHH or multimeric form thereof is administered concomitantly with the risk of exposure of a subject to infectious C. difficile microorganisms and production of toxin B (i.e., the target of the anti-TcdB VHH or multimeric form thereof), or slightly after the risk of exposure. For example, an anti-TcdB VHH or multimeric form thereof is administered to a subject at the moment that the subject contacts, enters, or passes through an environment (e.g., room, hallway, building, and field) containing a risk of exposure to C. difficile microorganisms.

The methods described herein provide treating or protecting a subject from intoxication and disease (and the symptoms thereof) caused by the toxin B protein produced by C. difficile bacteria that have infected the subject. In accordance with the method, one or more anti-TcdB VHHs or multimeric forms thereof and, optionally, an anti-tag antibody(ies) as described herein are administered to the affected or at risk subject. Administration ameliorates, reduces, or alleviates the severity of one or more the symptoms of the C. diff. -induced disease or condition. The presence, absence, or severity of symptoms is measured, for example, using physical examination, tests and diagnostic procedures known and practiced in the art. In certain embodiments, the presence, absence and/or level of C. diff. and/or its produced toxin B protein are measured using methods known and employed in the art. Symptoms or levels of the toxin B protein can be measured at one or more time points (e.g., before, during and after treatment, or any combination thereof) during the course of treatment with an anti-TcdB VHH or multimeric form thereof to determine if the treatment is effective. A decrease, reduction, or no change in the levels of the toxin B protein, or in the severity of symptoms associated therewith indicates that treatment is effective, and an increase in the level of toxin B, or in the severity of symptoms in a subject indicates that treatment is not effective. In various embodiments, the symptoms and levels of toxin B are measured using methods known and employed in the art. Specific, yet nonlimiting, symptoms that are monitored in subjects with C. difficile infection may include watery diarrhea, cramping/tenderness (mild symptoms); severe watery diarrhea, colitis, severe abdominal cramping/tenderness, rapid heart rate, fever, blood in stool, dehydration, loss of appetite and weight, swollen abdomen, kidney failure and increase leukocyte count (severe symptoms). Treatment or protection from intoxication by toxin B is assessed as increased survival and reduction, alleviation, or prevention of symptoms. Methods, compositions and kits involving the use of the anti-TcdB VHHs or multimeric forms thereof described herein decrease and alleviate the symptoms of C. diff. infection and toxin B production and also improve survival from exposure to the C. diff. bacteria themselves.

### Modes of Delivery of Anti-TcdB VHHs for Treatment of CDI

The anti-TcdB VHHs or multimeric forms thereof described herein can serve as effective parenteral therapeutics, e.g., in several animal models and in patients. These anti-TcdB VHHs or multimeric forms may substantially improve functional stability in the harsh proteolytic conditions of the GI tract. A number of different modes of delivery may be employed to administer these VHH molecules, such as enteric delivery, intravenous or subcutaneous delivery. In addition, suitable oral delivery modes and systems as described herein may circumvent or overcome loss of protein stability associated with the provision of protein therapeutics in the environment of the GI tract. In embodiments, the anti-TcdB VHHs and multimeric forms thereof are employed in oral, enteric, or parenteral delivery systems and offer more stable therapeutics for treating TcdB intoxication and CDI caused by C. difficile infection.

### Spirulina-Based Oral or Enteric Treatment of CDI

In an aspect, the anti-TcdB VHHs or multimeric forms thereof described herein are delivered orally or enterically using genetically modified cyanobacteria or Spirulina, a blue-green alga, as described, for example, in U.S. Pat. No. 10,131,870 (Lumen Biosciences) and WO 2016/040499 A1, the contents of which are incorporated by reference herein in their entireties. In an embodiment, Spirulina algae are non-naturally occurring, stable transformants comprising at least one introduced targeted nucleotide mutation in their genome. In embodiments, the Spirulina is Arthrospira platensis or Arthrospira maxima. In some embodiments, the Spirulina is Arthrospira platensis NIES-39 or Arthrospira sp. PCC 8005. Spirulina modified to contain one or more stable, targeted mutations are suitable for molecularly engineering to harbor and express polynucleotides encoding the anti-TcdB binding polypeptides (anti-TcdB VHHs) as described herein and for producing and manufacturing products for therapeutic use. In an embodiment, the Spirulina organisms are engineered to express protease stable anti-TcdB VHHs. In an embodiment, such engineered Spirulina organisms are orally delivered to a subject in need for the treatment of CDI in the subject. In another embodiment, such engineered Spirulina organisms are enterically delivered to a subject in need for the treatment of CDI in the subject. Protease-stable anti-TcdB VHHs that neutralize TcdB toxin may thus be provided to the subject. Engineered Spirulina have been demonstrated be capable of expressing functional VHHs directed against an enteric pathogen at high levels (e.g., >1% dry mass); therapeutic efficacy of such a VHH product expressed by engineered Spirulina has also be shown.

### Probiotic E. coli Engineered to Secrete or Express TcdB-Binding VHH

The human gastrointestinal (GI) tract contains a complex symbiotic microbiota that is estimated to comprise more than 40,000 species and in some regions more than 1011 organisms. The microbiota of the GI tract aids in maintaining immune homeostasis in the gut-associated lymphoid tissues, optimizing nutritional uptake, and supporting gut development. The entire intestinal epithelium is overlaid with a thick mucus layer, which, in conjunction with an effective immune system, keep the enormous bacterial load strictly sequestered on the luminal side of the gut, thus preventing penetration across the epithelial barrier. (See, e.g., K. Gronbach et al., 2010, Infect Immun, 78(7):3036-3046).

The importance of the cross talk between the microbiota, intestinal epithelial cells, and the innate and adaptive components of the immune system is shown by a variety of intestinal pathological conditions, including Crohn's disease, ulcerative colitis, pouchitis, irritable bowel syndrome, necrotizing enterocolitis (NEC), and CDI. Immature or genetically compromised immunity results in exaggerated intestinal inflammation or disruption or altered composition of the intestinal mucosa, which, in turn, disturbs the homeostasis between a human host and its intestinal microbial symbionts. Pathological events, such as infection by pathogens, e.g., C. diffiicile, change the relative balance between beneficial and aggressive enteric symbionts, convert beneficial bacteria into pathogens, or select for new opportunistic pathogens. A qualitatively and quantitatively changed gastrointestinal microbiota, often termed small bowel bacterial overgrowth (SIBO) or dysbiosis, may contribute substantially to local chronic inflammation in a vicious cycle and provoke bacterial translocation that leads to fatal sepsis.

The foregoing provides the rationale for selective therapeutic manipulation of the abnormal microbiota by probiotics as therapeutics for the intestinal diseases. In general, probiotics are viable microorganisms with beneficial physiological or therapeutic activities. Various in vitro and animal studies with probiotics, including Escherichia coli strain Nissle 1917, have demonstrated the capacity of probiotics to reduce intestinal inflammation, to strengthen the intestinal barrier against pathogens, to increase the host innate immune functions, or to prevent adherent and invasive E. coli strains from adhering to and invading human intestinal epithelial cells. (Id.) Limited clinical trials using E. coli strain Nissle 1917 or other microorganisms have suggested that this therapeutic strategy can be efficacious in patients with GI tract conditions such as chronic idiopathic inflammatory bowel diseases (IBD), irritable bowel syndrome (IBS), and NEC.

In an embodiment, strains of the probiotic E. coli Nissle are molecularly engineered to express and secrete functional anti-TcdB VHH polypeptides, or to express the polypeptides produced by these microorganism on their surface. Such anti-TcdB VHH-producing probiotic microorganisms provide an oral therapeutic or treatment modality for delivering the anti-TcdB VHHs to the GI tract to treat C. difficile pathologies and their symptoms, such as CDI. By way of example, E. coli Nissle strains were engineered to express VHH polypeptides that neutralized Shiga toxin; these microorganisms were shown to establish in the GI tract of animal models where they continued to produce the anti-Shiga toxin VHHs. In the same manner, the anti-TcdB binding and/or neutralizing VHHs described herein and expressed by E. coli Nissle strains may be utilized to provide a safe oral therapy for CDI.

### Encapsulated Delivery of Anti-TcdB VHH

In another embodiment, encapsulated delivery and packaging technologies that allow orally administered protein therapeutics to pass safely through the stomach and release in the small or large intestine may be used to deliver anti-TcdB VHHs as described herein in the treatment of C. difficile infection, CDI and symptoms thereof. Encapsulation and enteric coating techniques and processes commonly known and used in the art are suitable for delivering anti-TcdB VHHs. In embodiments, nanoparticle-based delivery of drugs and biologics and enteric coating of nanoparticles have been described by J. K. Patra et al., 2018, J. Nanobiotech, 16, Art. No. 71 (doi.org/10.1186/s12951-018-0392-8); US Publication No. 20200129444, the contents of which are incorporated by reference herein. Nanoparticles engineered to deliver protease-resistant, TcdB-binding and/or neutralizing VHHs may be introduced into the colons of animal models. The animals may be assessed using methods and protocols known and used in the art to determine that these animals are protected from CDI pathology.

### mRNA Delivery of TcdB VHH

In an embodiment, a polynucleotide encoding an anti-TcdB VHHs or multimeric form thereof as described herein, in particular, mRNA, in the form of lipid nanoparticles is used to deliver these anti-TcdB binding agents and produce effective and long-lasting antibody titers in subjects who are passively immunized with the mRNA-nanoparticles. In a particular embodiment, the mRNA, which is otherwise unmodified, may be codon optimized to afford efficient expression of an anti-TcdB VHH or multimeric form thereof from the transcribed mRNA. It has been reported that exogenous mRNA has the ability to instruct cells to produce VHHs, as well as other types of antibodies. See, M. Thran et al., 2017, EMBO Mol. Medicine, online publication no. DOI 10.15252/emmm.201707678. The advantages of using mRNA for passive immunization are appreciated by those in the art. (See, M. Thran et al., Id.). mRNA-based approaches for therapeutics may be safer and more cost effective compared with DNA-based approaches. Because mRNA does not integrate into a host's DNA and is more transient in nature, mRNA-based protein expression is considered to be easier to control for protein expression.

### Substantially Identical Amino Acid and Nucleotide Sequences for VHHs

There is a large body of information in the literature supporting the fact that closely related antibody (Ab) sequences are capable of performing the same binding and therapeutic functions such that this is now generally accepted by those with ordinary skill in the art of immunological sciences. The creation of Abs with small numbers of amino acid sequence variations occurs naturally within mammals and some other animal species during the process of ‘affinity maturation’ in which Ab-producing cells that bind a newly encountered antigen (Ag) are expanded, and their progeny cells contain random mutations within portions of the Ab coding DNA that results in new, related Ab sequences. The cells expressing Abs that have gained improved binding properties for the new Ag are then selected and expanded, thereby increasing the amount of the improved antibody in the animal. This process continues through multiple generations of mutation and selection until Abs with greatly improved binding properties result, thus providing, for example, better immunity against pathogens expressing or possessing the new Ag. The process of Ab affinity maturation demonstrates that related, yet not identical, Ab amino acid sequences can possess similar target binding properties and perform similar therapeutic functions in vivo.

Example 1 herein provides anti-TcdB VHH antibodies having related sequences that perform similar functions and provide similar therapeutic benefits. The Abs described herein are heavy-chain only, single domain VHH antibodies, which are generated in camelid alpacas, which have been reported to be convenient sources of camelid VHH antibodies (See. e.g., Maass, D. R. et al., 2007, J. Immunol. Methods, 324:13-25). Briefly, alpacas are immunized with a selected C. difficile toxin B antigen (TcdB Ag) multiple times to permit the animal to undergo affinity maturation of the anti-TcdB VHHs that are produced. Anti-TcdB VHHs are then isolated and the encoding DNA selected for expression of soluble VHHs that bind TcdB Ag and have potential therapeutic or diagnostic properties. During this process, many examples of closely related anti-TcdB binding VHHs are isolated, which are distinctive, and which are presumably intermediates that result from the affinity maturation process which occurs during anti-TcdB VHH production in alpaca lymphocytes. These related anti-TcdB VHHs are screened for binding to TcdB Ag, and the most promising members of homology groups of TcdB-binding VHHs are identified and become lead candidates for further development.

Similar to all mammalian antibodies, VHHs comprise four, well-conserved ‘framework’ regions (FRs) which are important in forming the antibody structure. Between the FRs (FR1, FR2, FR3 and FR4) are three much less well-conserved CDRs or hypervariable regions (CDR1, CDR2 and CDR3) which principally interact with and bind to antigenic determinants or epitopes on antigens (Ags), such as TcdB. The CDR sequences vary widely so as to interact and bind to epitopes of Ags. The third CDR, CDR3, is generally the longest in sequence and is most diverse of the CDRs within VHHs, both in size and sequence. By way of nonlimiting example, CDR3 in VHHs can range in size from about 7 to about 28 amino acid residues. The CDR3 regions of VHHs generated in the same alpacas and selected for binding to a common target Ag are highly similar in size (number of amino acids comprising CDR3) and can vary in their amino acid identities. Without being bound by theory, VHHs and CDR3 regions that bind to the same TcdB target Ag are considered to have resulted from affinity maturation of a common precursor VHH within the animal and are classified as a ‘homology group’. Individual VHHs within a homology group are classified by their binding to a target Ag, and the members of the VHH homology group are able to ‘compete’ with each other for binding to the Ag, thus demonstrating that they bind to the same region on the target Ag. In VHH molecules, the CDRs (CDR1, CDR2 and CD3) play a role in the ability of a VHH to bind to the target Ag, e.g., TcdB, in conjunction with CDR1 and CDR2.

Since the FRs are critical for maintaining the structure of a VHH and the positioning of the CDRs for binding to the target Ag, the FRs of VHHs typically do not vary extensively in sequence. However, some VHH FR amino acid sequence variation is permissible, particularly in cases in which an amino acid substitution involves the replacement or substitution of one amino acid with another amino acid having similar properties (e.g., similarity in being charged or uncharged), i.e., a conservative substitution. Such conservative changes in FRs can often be found naturally within VHHs that have undergone affinity maturation in an animal. Similar to the case with FRs, VHH CDRs also typically do not vary extensively in amino acid sequence or type so as not to compromise their ability to specifically bind to Ag. As would be appreciated by one skilled in the art, an estimation of the extent of amino acid sequence variation that can be tolerated within VHHs without compromising their Ag binding ability can be made by observing the variation that occurs naturally within affinity-matured homology groups of VHHs isolated from the same types of animals and which bind to the same Ag.

An illustrative example of VHH sequence relatedness and the retention of common antigen binding properties is shown in FIG. 6A. The relatedness among the amino acid sequences of the anti-TcdB VHH polypeptides described in Example 1 herein is represented as a phylogram or phylogenetic tree. These sequences form homology groups and all of the anti-TcdB VHHs bind to TcdB despite variation in amino acid sequence identity, e.g., from approximately 78% to approximately 91% identity. In an embodiment, sequence variation is particularly acceptable in the CDR regions, e.g., CDR1, CDR2, and/or CDR3, while the feature of VHH binding to antigen TcdB is maintained. In an embodiment, amino acid sequence variation results from conservative amino acid substitutions in a VHH sequence. In an embodiment, the conservative amino acid substitutions are in one or more CDR sequences of the VHH polypeptide. In an embodiment, the conservative amino acid substitutions are in one or more FR sequences of the VHH polypeptide. In an embodiment, the conservative amino acid substitutions are in one or more CDR sequences and in one or more FR sequences of the VHH polypeptide.

Another example evidencing that VHH sequence variation is acceptable within related VHHs having the same Ag binding characteristics is described in Tremblay et al., 2013, Infect Immun 81:4592-4603. In this report, 11 VHH sequences comprise a large homology group with closely related CDR3 sequences, and the unusual property of cross-specific binding to two different Shiga toxins, Stx1 and Stx2. Two of the more distantly related VHH members of this homology group are characterized as having common Ag binding characteristics. These two related VHHs were found to have 32 amino acid changes in the total VHH sequence of 120 or 121 residues. Thus, a 26% variation in amino acid sequence did not adversely affect the common Ag binding properties of the VHH proteins.

### Kits

Provided herein are kits for the treatment or prevention of C. difficile infection or disease. In some embodiments, the kit includes an effective amount of one or more anti-TcdB VHHs or multimeric forms thereof as described herein, in unit dosage form. In an embodiment, the kit further contains an anti-epitope tag antibody, in unit dosage form. In other embodiments, the kit includes a therapeutic or prophylactic composition containing an effective amount of one or more anti-TcdB VHHs or multimeric forms thereof, in unit dosage form. In still other embodiments, the kit includes a therapeutic or prophylactic composition containing an effective amount of one or more anti-TcdB VHHs or multimeric forms thereof, and an anti-epitope tag antibody, in unit dosage form. In some embodiments, the kit comprises a device, e.g., an automated or implantable device for subcutaneous delivery; an implantable drug-eluting device, or a nebulizer or metered-dose inhaler, for dispersal of the composition or a sterile container which contains a pharmaceutical composition. Non-limiting examples of containers include boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, a pharmaceutical composition is provided together with instructions for administering the pharmaceutical composition containing one or more anti-TcdB VHHs or multimeric forms thereof, or one or more anti-TcdB VHHs or multimeric forms thereof and an anti-epitope tag antibody, to a subject having or at risk of contracting or developing an infection or disease or pathology, and/or the symptoms thereof, associated with infection by C. difficile and toxin B production by C. diffiicile. The instructions will generally include information about the use of the composition for the treatment or prevention of an infection and intoxication by the C. difficile bacteria and toxin B proteins that they produce. In other embodiments, the instructions include at least one of the following: description of the therapeutic/prophylactic agent; dosage schedule and administration for treatment or prevention of infection, disease or symptoms thereof caused by one or more of C. difficile bacteria and/or toxin B proteins that they produce; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

In another aspect, a kit is provided for treating a subject exposed to, intoxication by, or at risk for exposure to or intoxication by C. difficile toxin B, in which the kit includes a pharmaceutical composition for treating a subject at risk for exposure to or who is exposed to C. diff. and/or toxin B produced by C. diff. , and the pharmaceutical composition includes at least one recombinant anti-TcdB VHH or multimeric form thereof, such that the anti-TcdB VHH or multimeric form thereof neutralizes C. diff. TcdB, thereby treating the subject for exposure to the C. diff. disease agent; a container; and, instructions for use. In various embodiments, the instructions for use include instructions for a method for treating a subject at risk for exposure to, exposed to, or intoxicated by the C. diff. disease agent using the pharmaceutical composition.

The practice of the present aspects and embodiments described herein employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the aspects and embodiments described herein, and, as such, may be considered in making and practicing the same. Particularly useful techniques for particular embodiments will be discussed in the Examples that follow.

### EXAMPLES

### Example 1—Anti-C. difficile Toxin B (TcdB)-Binding VHHs

Presented in Example 1 are the amino acid and encoding polynucleotide (nucleic acid) sequences of C. difficile toxin B (TcdB)-binding VHH polypeptides (anti-TcdB VHHs) as described herein. The amino acids comprising the Complementarity Determining Regions (CDRs) of each of the anti-TcdB VHHs are designated in each VHH polypeptide as follows: CDR1 is designated by a single underline; CDR2 is designated by a double underline; and CDR3 is designated in bold with a single underline.

JZS-A2a (XAF-1) VHH amino acid sequence
(SEQ ID NO: 1)
QVQLAESGGGLVQAGGSLRLSCAASGSVYTFMGWYRQAPGKTRELVAGISGGTITKYADS
VKGRFIISRDSPKNTIYLQMNELKVEDTGVYYCNAGDTIAQAMGTRRFPFDRWGQGTQVT
VAS
JZS-A2a (XAF-1) polynucleotide sequence
(SEQ ID NO: 2)
CAGGTGCAGCTCGCGGAGTCGGGAGGAGGCTTGGTGCAGGCTGGGGGGTCTCTGAGACTC
TCCTGTGCAGCCTCTGGAAGCGTGTATACTTTTATGGGCTGGTACCGCCAGGCTCCAGGG
AAGACGCGCGAATTGGTCGCAGGGATTTCAGGTGGTACGATCACAAAATATGCAGACTCC
GTGAAGGGCCGATTCATCATCTCCAGAGACAGCCCCAAGAACACAATCTATCTGCAAATG
AACGAGCTAAAAGTTGAAGACACAGGCGTGTATTACTGTAATGCAGGCGACACCATTGCA
CAGGCTATGGGGACGCGGAGGTTTCCGTTCGACCGCTGGGGCCAGGGGACCCAGGTCACC
GTCGCCTCA
JZS-B2 (XAF-4) VHH amino acid sequence
(SEQ ID NO: 3)
QVQLAESGGGLVQAGGSLRLSCVGSGTSFPRNYMGWYRQAPGKQRELVAAISHDGNVEYA
DSVKGRFTISRGNFVNTVALQMNSLKSEDTAVYYCKLVTLRRDEYWGQGTQVTVSS
JZS-B2 (XAF-4) polynucleotide sequence
(SEQ ID NO: 4)
CAGGTGCAGCTCGCGGAGTCGGGAGGAGGCTTGGTGCAGGCTGGGGGGTCTCTGAGACTC
TCCTGTGCAGCCTCTGGAAGCGTGTATACTTTTATGGGCTGGTACCGCCAGGCTCCAGGG
AAGACGCGCGAATTGGTCGCAGGGATTTCAGGTGGTACGATCACAAAATATGCAGACTCC
GTGAAGGGCCGATTCATCATCTCCAGAGACAGCCCCAAGAACACAATCTATCTGCAAATG
AACGAGCTAAAAGTTGAAGACACAGGCGTGTATTACTGTAATGCAGGCGACACCATTGCA
CAGGCTATGGGGACGCGGAGGTTTCCGTTCGACCGCTGGGGCCAGGGGACCCAGGTCACC
GTCGCCTCA
JZS-B9 (XAF-5) VHH amino acid sequence
(SEQ ID NO: 5)
QLQLVESGGGLVQPGGSLRLSCTSARFSLINYAIGWFRQAPGQKREGVSLLTSGGATYYA
DSARDRFTISRDNARNTVYLQMNSLKPEDTAVYSCAAGPYSRTLVSRWKVGDGMEYWGKG
TLVTVSS
JZS-B9 (XAF-5) polynucleotide sequence
(SEQ ID NO: 6)
CAGTTGCAGCTCGTGGAGTCTGGGGGAGGCCTGGTGCAGCCTGGGGGTTCTCTGAGACTC
TCCTGTACATCTGCGAGATTCTCTTTGATTAATTATGCCATAGGCTGGTTCCGCCAGGCC
CCAGGACAGAAGCGCGAGGGGGTCTCACTACTTACTAGTGGTGGTGCTACATACTATGCT
GACTCCGCGAGGGACCGATTCACCATCTCCAGAGACAACGCCAGGAACACGGTGTATTTG
CAAATGAACAGCCTGAAACCTGAGGACACGGCCGTTTATTCGTGTGCAGCAGGGCCCTAT
TCCCGAACCTTAGTCTCACGTTGGAAGGTGGGGGACGGCATGGAGTACTGGGGCAAAGGG
ACCCTAGTCACCGTCTCCTCA
JZS-C2 (XAF-6) VHH amino acid sequence
(SEQ ID NO: 7)
QVQLAESGGGLVQPGGSLRLSCAASGFTSNSYYIGWFRQAPGKGREAVSSISSSGGSPNY
ANAVKGRFTITRDNANNTVYLQMDNLKPEDTAVYYCAASKFPLTTMASNRYHYWGQGTQV
TVSS
JZS-C2 (XAF-6) polynucleotide sequence
(SEQ ID NO: 8)
CAGGTGCAGCTCGCGGAGTCGGGCGGAGGCTTGGTGCAGCCTGGGGGTTCTCTGAGACTC
TCCTGTGCAGCCTCTGGATTCACTTCGAATTCTTATTACATAGGCTGGTTCCGCCAGGCC
CCAGGGAAGGGGCGCGAGGCGGTCTCAAGTATTAGTAGTAGTGGAGGTAGCCCTAACTAT
GCGAATGCCGTGAAGGGCCGATTCACCATAACCAGAGACAACGCCAACAACACGGTCTAT
CTGCAAATGGACAACCTGAAACCTGAGGACACGGCCGTTTATTACTGCGCAGCATCGAAG
TTTCCGCTAACCACTATGGCGTCCAACCGATATCATTACTGGGGTCAGGGGACCCAGGTC
ACCGTCTCCTCA
JZS-C10 (XAF-9) VHH amino acid sequence
(SEQ ID NO: 9)
QLQLVETGGLVQAGGSLRLSCVGSGRGPGINVMGWYRQAPGTERELVATWQTGGTTNYAD
SVKGRFTISRDNLKNTVSLQMDSLKPEDTAVYYCYLKKWRDEYWGQGTQVTVSS
JZS-C10 (XAF-9) polynucleotide sequence
(SEQ ID NO: 10)
CAGTTGCAGCTGGTGGAGACGGGAGGCTTGGTGCAGGCTGGGGGGTCTCTGAGACTCTCC
TGTGTAGGCTCTGGAAGAGGCCCCGGGATCAATGTCATGGGCTGGTACCGCCAGGCTCCA
GGGACTGAGCGCGAGTTGGTCGCAACTTGGCAAACCGGTGGTACCACAAACTATGCAGAC
TCCGTGAAGGGCCGATTCACCATCTCCAGAGACAACCTCAAGAACACGGTGTCGCTGCAA
ATGGACAGTCTGAAACCTGAGGACACAGCCGTCTATTACTGCTATCTGAAAAAATGGAGA
GATGAGTATTGGGGCCAGGGGACCCAGGTCACCGTCTCCTCA
JZS-E4 (XAF-10) VHH amino acid sequence
(SEQ ID NO: 11)
QVQLVESGGGLAQAGGSLRLSCAASGSSFSMNVMGWYRQPPGKQRELVATIRSDGITNYA
ESVKGRFTISRDNVKNTVHLEMNRLKAEDTAVYYCFHGRARTGNNADLGSWGQGTQVTVS
S
JZS-E4 (XAF-10) polynucleotide sequence
(SEQ ID NO: 12)
CAGGTGCAGCTGGTGGAGTCGGGTGGAGGCTTGGCGCAGGCTGGGGGGTCTCTGAGACTC
TCCTGTGCAGCCTCTGGAAGTAGCTTCAGCATGAATGTCATGGGCTGGTACCGCCAGCCT
CCAGGGAAGCAGCGCGAGTTGGTCGCGACTATTCGTAGTGATGGTATCACAAACTATGCA
GAGTCCGTGAAGGGCCGATTCACCATCTCCAGAGACAACGTCAAGAACACAGTGCATCTG
GAAATGAACAGGCTGAAAGCTGAAGACACAGCCGTATATTACTGCTTTCATGGCCGGGCC
CGCACAGGGAATAATGCTGACTTGGGTTCTTGGGGCCAGGGGACCCAGGTCACCGTCTCC
TCG
JZS-E6 (XAF-11) VHH amino acid sequence
(SEQ ID NO: 13)
QVQLAETGGGLVQAGGSLRLSCAASGRLSERIFMISTMAWYRQVPGKQRELVAEISRLGR
ANYSDSVTDRFIISRDNTKNTVDLQMNSLKPEDTAVYYCNLKPFVDNYRGPGTQVTVSS
JZS-E6 (XAF-11) polynucleotide sequence
(SEQ ID NO: 14)
CAGGTGCAGCTGGCGGAGACGGGTGGAGGCTTGGTGCAGGCTGGGGGGTCTCTGAGACTC
TCCTGTGCAGCTTCGGGAAGGCTCTCGGAAAGGATCTTCATGATCAGTACGATGGCCTGG
TACCGCCAGGTTCCAGGGAAGCAGCGCGAGTTGGTCGCAGAAATTTCGCGACTTGGTAGG
GCCAACTATTCAGACTCCGTGACGGACCGATTCATCATCTCCAGAGACAACACCAAGAAC
ACGGTGGATCTACAAATGAACAGCCTGAAGCCTGAGGACACAGCCGTCTATTACTGCAAT
CTTAAACCCTTCGTCGACAACTACCGGGGCCCGGGGACCCAGGTCACCGTCTCCTCT
JZS-F6 (XAF-12) VHH amino acid sequence
(SEQ ID NO: 15)
QLQLAESGGGLVQPGGSLRLSCAASGITFSNVAMSWVRQAPGKGLEWVSTISTGGSSTSY
LDSVKSRFTISRDNAKKTVYLQMNSLKPEDTAVYYCVKGPKYSATIRRPEGQGTQVTVSS
JZS-F6 (XAF-12) polynucleotide sequence
(SEQ ID NO: 16)
CAGTTGCAGCTCGCGGAGTCCGGCGGAGGCTTGGTGCAGCCTGGGGGGTCTCTGAGACTC
TCCTGTGCAGCCTCTGGAATCACCTTCAGTAACGTTGCCATGAGCTGGGTCCGCCAGGCT
CCAGGAAAGGGGCTCGAGTGGGTCTCAACTATTAGTACGGGCGGTAGTAGTACAAGCTAT
TTAGACTCCGTGAAGAGCCGATTGAGCATCTCCAGAGACAACGCCAAGAAGACGGTGTAT
CTGCAAATGAACAGTCTGAAACCTGAGGACACGGCCGTGTATTACTGTGTAAAAGGGCCC
AAGTATTCCGCTACAATCCGTCGTCCTGAGGGCCAGGGGACCCAGGTCACCGTCTCCTCA
JZS-H4 (XAF-13) VHH amino acid sequence
(SEQ ID NO: 17)
QLQLVESGGGLVQPGGSLRLSCVASGFNFSVQIMSWVRQAPGKGLEWVSAISTGGASKSY
ADFAKGRFTISRDNAKNTLYLQMNSLQLEDTAVYFCSKGPRTWINSSPRGQGTQVTVSS
JZS-H4 (XAF-13) polynucleotide sequence
(SEQ ID NO: 18)
CAGTTGCAGCTCGTGGAGTCCGGTGGAGGCTTGGTGCAGCCTGGGGGGTCTCTGAGACTC
TCCTGTGTAGCCTCTGGATTCAACTTCAGTGTGCAGATTATGAGCTGGGTCCGCCAGGCT
CCAGGAAAGGGGCTCGAGTGGGTCTCAGCTATTAGTACTGGTGGCGCTAGCAAAAGTTAT
GCAGACTTCGCGAAGGGCCGATTCACCATCTCCAGAGACAACGCCAAGAACACGCTGTAT
CTGCAAATGAACAGCCTACAACTCGAGGACACGGCCGTGTATTTTTGTTCTAAGGGTCCG
AGGACTTGGATCAATTCTAGTCCCCGGGGCCAGGGGACCCAGGTCACCGTCTCCTCG
JZS-H9 (XAF-14) VHH amino acid sequence
(SEQ ID NO: 19)
QVQLVESGGGLVQPGGSLTINCTVSGTAFSLDTMTWYRQAPGKQRELAADISSSGASNYL
ASVKGRFTISRDNAKSALYLQMNSLKPEDTGTYYCYRGRVRGVWPLDSGMMYWGKGTLVT
VSS
JZS-H9 (XAF-14) polynucleotide sequence
(SEQ ID NO: 20)
CAGGTGCAGCTGGTGGAGTCCGGTGGAGGCTTGGTGCAGCCTGGGGGCTCTCTGACAATC
AACTGTACAGTCTCTGGAACCGCGTTCAGTCTCGATACCATGACCTGGTACCGCCAGGCT
CCAGGGAAGCAGCGCGAGTTGGCCGCCGATATTAGTAGTAGTGGTGCCTCAAACTATTTA
GCCTCCGTGAAGGGCCGATTCACCATCTCCAGAGATAACGCCAAGAGCGCTCTGTATCTG
CAAATGAACAGCCTGAAACCTGAGGACACAGGCACATATTATTGCTATAGAGGGCGAGTG
CGGGGAGTCTGGCCGTTGGACAGCGGCATGATGTACTGGGGTAAAGGGACCTTGGTCACC
GTCTCCTCA
JZT-F7 (XAG-1) VHH amino acid sequence
(SEQ ID NO: 21)
QVQLVETGGGLVQAGGSLRLSCAASGSILSSMGWYRQAPGNQREFVASISRTGATDYADS
VAGRFIISRDRGKNTVLALQMDSLKPEDTAVYYCNAGLGMGDPRRPGPWWGQGTQVTVSS
JZT-F7 (XAG-1) polynucleotide sequence
(SEQ ID NO: 22)
CAGGTGCAGCTCGTGGAGACTGGAGGAGGCTTGGTGCAGGCTGGGGGGTCTCTGAGACTC
TCCTGTGCAGCCTCTGGAAGCATCCTCAGTTCCATGGGCTGGTACCGCCAGGCTCCAGGA
AACCAGCGCGAGTTCGTCGCGTCTATTTCTCGTACTGGGGCGACGGACTATGCAGACTCC
GTGGCGGGCCGATTCATCATCTCTAGGGACAGAGGCAAGAACACGGTATTAGCGCTGCAA
ATGGACAGCCTGAAACCTGAGGACACAGCCGTCTATTACTGTAATGCAGGATTAGGAATG
GGGGACCCGCGACGGCCCGGTCCGTGGTGGGGCCAGGGAACCCAGGTCACCGTCTCCTCA
JZT-G9 (XAG-7) VHH amino acid sequence
(SEQ ID NO: 23)
QVQLAETGGGLVQAGGSLRLSCVSSERNPGINAMGWYRQAPGSQRELVAVWQTGGSLSYA
DSVKGRFTISRDNLKNTVYLQMNSLKPEDAAVYYCYLKKWRDQYWGQGTRVTVSS
JZT-G9 (XAG-7) polynucleotide sequence
(SEQ ID NO: 24)
CAGGTGCAGCTCGCGGAGACTGGTGGAGGCTTGGTTCAGGCAGGCGGGTCTCTGAGACTC
TCCTGTGTAAGCTCTGAAAGAAATCCCGGGATCAATGCCATGGGCTGGTATCGCCAGGCT
CCAGGGAGTCAGCGCGAGTTGGTCGCAGTTTGGCAAACCGGTGGTAGCCTAAGCTATGCA
GACTCCGTGAAGGGTCGATTCACCATCTCCAGAGACAACCTCAAGAACACGGTGTATCTG
CAAATGAACAGTCTGAAACCTGAGGAGGCAGCCGTCTATTATTGTTATCTGAAAAAGTGG
AGAGATCAGTATTGGGGCCAGGGGACCCGGGTCACCGTCTCCTCA

Framework region (FR) sequences of the representative C. difficile toxin B (TcdB)-binding VHH polypeptides (anti-TcdB VHHs) described herein are presented below. Without intending to be limited by theory, in some cases, amino acid residue(s) close or directly adjacent to amino acid residue(s) at the boundaries of FR/CDR regions may be included in the FR sequence or in the CDR sequence, or such residues may vary, e.g., amino acid substitutions such as conservative substitutions, among the VHH polypeptides without adversely affecting the TcdB binding or function of the VHH polypeptides.

```
JZS-A2a (XAF-1):
(SEQ ID NO: 72)
FR1: QVQLAESGGGLVQAGGSLRLSCAAS;
  (SEQ ID NO: 73)
FR2: MGWYRQAPGKTRELVAGI;
(SEQ ID NO: 74)
FR3: YADSVKGRFIISRDSPKNTIYLQMNELKVEDTGVYYC;
  (SEQ ID NO: 75)
FR4: WGQGTQVTVAS.
JZS-B2 (XAF-4):
  (SEQ ID NO: 76)
FR1: QVQLAESGGGLVQAGGSLRLSCVGS;
  (SEQ ID NO: 77)
FR2: MGWYRQAPGKQRELVAAI;
  (SEQ ID NO: 78)
FR3: YADSVKGRFTISRGNFVNTVALQMNSLKSEDTAVYYC;
  (SEQ ID NO: 79)
FR4: WGQGTQVTVSS.
JZS-B9 (XAF-5):
  (SEQ ID NO: 80)
FR1: QLQLVESGGGLVQPGGSLRLSCTSA;
  (SEQ ID NO: 81)
FR2: IGWFRQAPGQKREGVSLL;
  (SEQ ID NO: 82)
FR3: YADSARDRFTISRDNARNTVYLQMNSLKPEDTAVYSC;
  (SEQ ID NO: 83)
FR4: WGKGTLVTVSS.
JZS-C2 (XAF-6):
  (SEQ ID NO: 84)
FR1: QVQLAESGGGLVQPGGSLRLSCAAS;
  (SEQ ID NO: 85)
FR2: IGWFRQAPGKGREAVSSI;
  (SEQ ID NO: 86)
FR3: YANAVKGRFTITRDNANNTVYLQMDNLKPEDTAVYYC;
  (SEQ ID NO: 79)
FR4: WGQGTQVTVSS.
JZS-C10 (XAF-9):
  (SEQ ID NO: 87)
FR1: QLQLVETGGLVQAGGSLRLSCVGS;
  (SEQ ID NO: 88)
FR2: MGWYRQAPGTERELVATW;
  (SEQ ID NO: 89)
FR3: YADSVKGRFTISRDNLKNTVSLQMDSLKPEDTAVYYC;
  (SEQ ID NO: 79)
FR4: WGQGTQVTVSS.
JZS-E4 (XAF-10):
  (SEQ ID NO: 90)
FR1: QVQLVESGGGLAQAGGSLRLSCAAS;
  (SEQ ID NO: 91)
FR2: MGWYRQPPGKQRELVATI;
  (SEQ ID NO: 92)
FR3: YAESVKGRFTISRDNVKNTVHLEMNRLKAEDTAVYYC;
  (SEQ ID NO: 79)
FR4: WGQGTQVTVSS.
JZS-E6 (XAF-11):
  (SEQ ID NO: 93)
FR1: QVQLAETGGGLVQAGGSLRLSCAAS;
  (SEQ ID NO: 94)
FR2: MAWYRQVPGKQRELVAEI;
  (SEQ ID NO: 95)
FR3: YSDSVTDRFIISRDNTKNTVDLQMNSLKPEDTAVYYC;
  (SEQ ID NO: 96)
FR4: GPGTQVTVSS.
JZS-F6 (XAF-12):
  (SEQ ID NO: 97)
FR1: QLQLAESGGGLVQPGGSLRLSCAAS;
  (SEQ ID NO: 98)
FR2: MSWVRQAPGKGLEWVSTI;
  (SEQ ID NO: 99)
FR3: YLDSVKSRFTISRDNAKKTVYLQMNSLKPEDTAVYYC;
  (SEQ ID NO: 100)
FR4: GQGTQVTVSS.
JZS-H4 (XAF-13):
  (SEQ ID NO: 101)
FR1: QLQLVESGGGLVQPGGSLRLSCVAS;
  (SEQ ID NO: 102)
FR2: MSWVRQAPGKGLEWVSAI;
  (SEQ ID NO: 103)
FR3: YADFAKGRFTISRDNAKNTLYLQMNSLQLEDTAVYFC;
  (SEQ ID NO: 100)
FR4: GQGTQVTVSS.
JZS-H9 (XAF-14):
  (SEQ ID NO: 104)
FR1: QVQLVESGGGLVQPGGSLTINCTVS;
  (SEQ ID NO: 105)
FR2: MTWYRQAPGKQRELAADI;
  (SEQ ID NO: 106)
FR3: YLASVKGRFTISRDNAKSALYLQMNSLKPEDTGTYYC;
  (SEQ ID NO: 83)
FR4: WGKGTLVTVSS.
JZT-F7 (XAG-1):
  (SEQ ID NO: 107)
FR1: QVQLVETGGGLVQAGGSLRLSCAAS;
  (SEQ ID NO: 108)
FR2: MGWYRQAPGNQREFVASI;
(SEQ ID NO: 109)
FR3: YADSVAGRFIISRDRGKNTVLALQMDSLKPEDTAVYYC;
  (SEQ ID NO: 79)
FR4: WGQGTQVTVSS.
JZT-G9 (XAG-7):
  (SEQ ID NO: 110)
FR1: QVQLAETGGGLVQAGGSLRLSCVSS;
  (SEQ ID NO: 111)
FR2: MGWYRQAPGSQRELVAV;
  (SEQ ID NO: 112)
FR3: YADSVKGRFTISRDNLKNTVYLQMNSLKPEDAAVYYC;
  (SEQ ID NO: 113)
FR4: WGQGTRVTVSS.
```

### Example 2—VHH-Display Library Preparation From Genes Expressed in Immunized Camelids (Alpacas)

In a general protocol, three alpacas were immunized with TcdB (100 μg) by five successive multi-site subcutaneous (SC) injections at three week intervals. For the first immunization, the adjuvant was alum/CpG and subsequent immunizations used alum. All alpacas achieved ELISA anti-TcdB titers of 1:1,000,000. Blood was obtained from the alpacas for peripheral blood lymphocyte (PBL) preparation seven days after the fifth immunization, and RNA was extracted using the RNEASY kit (Qiagen, Valencia, CA). VHH-display phage libraries were prepared as described in Maass, D. R., et al., 2007 Internatl J Parasitology, 37, 953-962 and Tremblay, J. M., et al., 2010 Toxicon 56, 990-998.

In a particular aspect, an alpaca that had been immunized with TcdB immunogen 10 years earlier was twice boosted with 100 ug of TcdB immunogen. Following these immunizations, immune peripheral blood lymphocytes (PBL) were obtained from the animal, and a VHH display library was produced from the immune PBLs. This anti-TcdB VHH display library was called JZM-3 and had a complexity of 1.1×107. Another anti-TcdB VHH display library, called JYH-3, was made by combining mRNA obtained from immune PBLs isolated from two other immunized alpacas. The two anti-TcdB VHH display libraries were subjected to a panning technique. (See, e.g., Mukherjee, J., et al., 2012 PLoS ONE, 7, e299411; Maass, D. R., et al., 2007 Internatl J Parasitology, 37, 953-962 and Tremblay, J. M., et al., 2010 Toxicon 56, 990-998). Most toxins, particularly large toxins such as those produced by C. diffiicile, become partially denatured on plastic. Because VHH-binding is highly dependent on protein conformation, anti-TcdB VHH libraries were extensively panned on antibody-captured TcdB to capture and select VHHs that bound to conformationally native TcdB protein. A VHH heterodimer containing two TcdB-neutralizing VHHs, called 5D and E3 (plasmid JXW-2) was used as a reference.

Twenty-five (25) clonally-unrelated, unique anti-TcdB VHH antibody families were generated from the two libraries screened by panning. Based on early screening data including binding and toxin neutralization data, 20 VHHs were selected for expression, purification and characterization. Additional panning and screening for TcdB-neutralizing VHHs was performed to employ capture panning with improved libraries to identify anti-TcdB VHHs that bound to neutralizing epitopes of TcdB. In addition, assays were carried out to identify TcdB-neutralizing VHHs that were resistant to resident proteases in the gastrointestinal tract (GI proteases). Such GI protease resistant anti-TcdB VHHs advantageously provide therapeutics for enteric delivery and effectiveness, for example, by employing Spirulina for delivery.

Following additional characterizations, 12 out of the 20 anti-TcdB VHHs that demonstrated the highest apparent affinity for TcdB and/or the highest potency for TcdB-neutralization were selected as representatives for further use and development. The protein sequences and encoding DNA sequences of the 12 anti-TcdB VHH antibodies are provided supra.

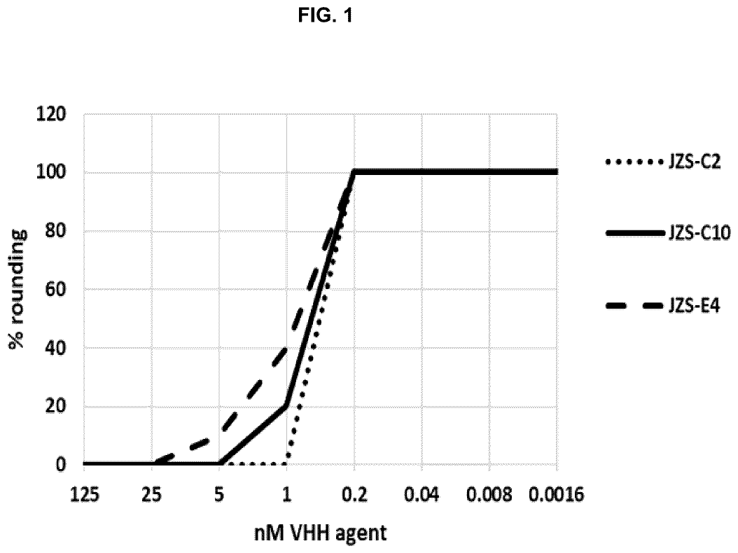

The anti-TcdB VHH were purified and their TcdB-neutralization potencies were examined. Briefly, to evaluate the neutralization activities of these VHHs, a dilution series of the individual VHHs were incubated with Vero cells in culture, and the cells were then intoxicated with TcdB. The rounding effects of toxin exposure following culturing the cells with anti-TcdB VHHs were assessed at various times after toxin was added to the cells. As shown in FIGS. 1-4, cell rounding was assessed at 5 hrs post-intoxication with 50 pg/ml of TcdB.

In summary, several hundred individual clones were screened for TcdB-binding; positive clones were characterized; and all new and unique TcdB-binding VHH coding DNAs were re-cloned into an expression vector for soluble protein expression and purification. The purified, quantified VHHs were characterized for their potencies to neutralize TcdB in a cell-based toxicity assay. The more potent neutralizing anti-TcdB VHHs were screened for their resistance to GI tract proteases using assays developed for enteric pharmacokinetic studies. Because panning on native TcdB was employed, as well as a more complex VHH display library prepared from optimally immunized alpacas, it was expected that ten or more unique new VHHs capable of binding TcdB with high affinity (sub-nM affinities) would be identified, and subsequently selected for use.

### Example 3—Binding of Anti-TcdB VHHs to TcdB

TcdB binding assays were performed to assess the binding activity of representative anti-TcdB VHH antibodies as described herein and in Yang, Z. et al., 2014, J Infect Disease, 210(6):964-972; doi: 10.1093/infdis/jiu196. In addition, the neutralization activity of the representative anti-TcdB VHH antibodies was assessed in neutralization assays as described herein and in Yang, Z. et al., 2014, Id. In particular, the anti-TcdB VHH antibodies were assayed for specific TcdB binding affinity (EC₅₀) and neutralizing potency (IC₅₀). The results are presented in Table 4 below. The anti-TcdB antibodies were found to bind to TcdB with varying affinities and/or neutralize TcdB activity with different potencies. In Table 4, the affinity and potency values are designated as “apparent” (rather than “intrinsic”), because they were determined experimentally using appropriate assays as commonly known and practiced in the art.

TABLE 4

| | | Apparent | | Apparent | |
|---|---|---|---|---|---|
| VHH | Vector | affinity, | | potency, | |
| name | name | (EC₅₀) | | (IC₅₀) | |
| JZS-A2a | XAF-1 | >25 | nM | >25 | nM |
| JZS-B2 | XAF-4 | 0.3 | nM | >125 | nM |
| JZS-B9 | XAF-5 | 0.2 | nM | 1 | nM |
| JZS-C2 | XAF-6 | 3 | nM | 0.5 | nM |
| JZS-C10 | XAF-9 | 0.1 | nM | 0.5 | nM |
| JZS-E4 | XAF-10 | 2 | nM | 1 | nM |
| JZS-E6 | XAF-11 | 0.8 | nM | 1 | nM |
| JZS-F6 | XAF-12 | 0.1 | nM | 0.5 | nM |
| JZS-H4 | XAF-13 | 0.2 | nM | 0.5 | nM |
| JZX-H9 | XAF-14 | 1 | nM | 15 | nM |
| JZT-F7 | XAG-1 | 3 | nM | 1 | nM |
| JZT-G9 | XAG-7 | 0.1 | nM | 3 | nM |

### Example 4—Neutralization of TcdB by Anti-TcdB VHHs

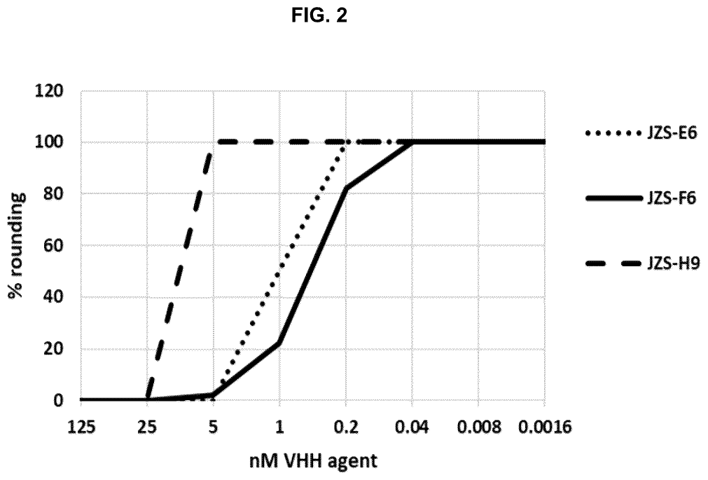

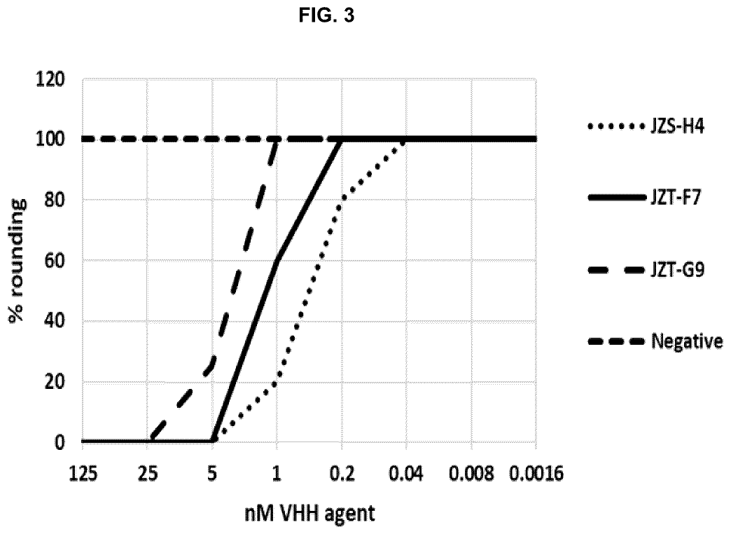

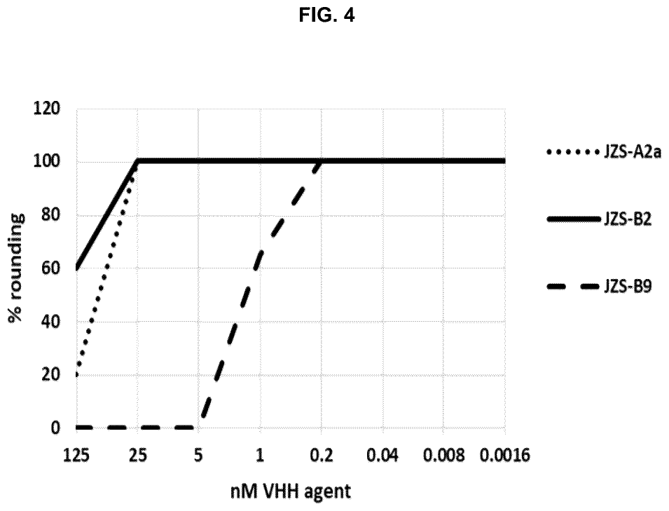

Neutralization assays as described in Example 5 infra (and as in Yang, Z. et al., 2014, J Infect Disease, 210(6):964-972; doi: 10.1093/infdis/jiu 196) were performed to assess the neutralization activity and apparent potency of the representative anti-TcdB VHHs described herein. The results of the assays are presented in Table 4 above and in FIGS. 1-4. In particular, the assays showed that the anti-TcdB VHHs JZS-C2, JZS-C10, and JZX-E4 (JZS-E4) displayed IC₅₀ TcdB-neutralization potency near 0.5 nM. (FIG. 1). The anti-TcdB VHH JZS-E6 displayed IC₅₀ TcdB-neutralization potency close to 1 nM; JZS-F6 displayed IC₅₀ TcdB-neutralization potency near 0.5 nM; and JZS-H9 displayed IC₅₀ TcdB-neutralization potency about 15 nM. (FIG. 2). The anti-TcdB VHH JZS-H4 displayed IC₅₀ TcdB-neutralization potency near 0.5 nM; JZT-F7 displayed IC₅₀ TcdB-neutralization potency close to 1 nM; and JZT-G9 displayed IC₅₀ TcdB-neutralization potency about 3 nM. (FIG. 3). The anti-TcdB VHHs JZS-A2a and JZS-B2 displayed minimal TcdB-neutralizing activity, while JZS-B9 displayed IC₅₀ TcdB-neutralization potency close to 1 nM. (FIG. 4). It will be appreciated that because neutralization assays were conducted in vitro, and slight variations may occur from assay to assay, the neutralization potency values are referred to herein as “apparent potency” values.

### Example 5—Materials and Methods

### Enzyme Linked Immunosorbent Assay (ELISA)

EIA/RIA 96 well high binding plates (Corning Costar) coated with 0.5-5 μg/ml of recombinantly produced toxin, e.g., rTcdB, overnight at 4° C. were used for immuno-binding assays (ELISA). Plates were washed 3 times with 1×PBS+0.1% Tween, followed by washing 3 times with 1×PBS. Washed plates were blocked (4-5% non-fat dry milk in 1×PBS+0.1% Tween) for 1 hour at room temperature (RT) with rocking. Serially diluted (1:5) VHH-TcdB binding molecules targeting C. difficile toxin B, diluted in blocking solution, were incubated for 1 hour at RT with rocking and washed as above. Equivalent control samples were spiked with a known amount of an irrelevant VHH for use as an internal standard.

Binding of the VHHs to recombinant toxin B coating the wells was detected at A450 nm using horse radish peroxidase (HRP)-labeled anti-E-tag antibody and an ELISA reader. Bound HRP was detected using 3,3′,5,5′-tetramethylbenzidine (TMB substrate, Sigma) and values were plotted as a function of the input VHH concentration. Illustratively, the plates were incubated with goat anti-E-tag-HRP conjugated antibody (Bethyl labs) diluted 1:5000 in blocking solution for 1 hour at RT with rocking and were washed as above before adding TMB microwell peroxidase substrate (KPL) to develop (incubated for 10-40 min). Development was stopped with 1M H₂SO₄ and the plates were read at 450 nm on an ELx808 Ultra Microplate Reader (Bio-Tek instruments), (Mukherjee, J. et al., 2012, PloS ONE 7:e29941). VHH levels in unknown samples were determined by comparison of their signals to those of internal standards as previously described (Mukherjee, J. et al., 2014, PLoS One 9:e 106422; Sheoran, A S et al., 2015, Infect Immun, 83:286-291; Moayeri, M. et al., 2016, Clin Vaccine Immunol, doi: 10.1128/cvi.00611-15; Sponseller, J K et al., 2014, J Infect Dis, doi:10.1093/infdis/jiu605; Tzipori, S. et al., 1995, Infect Immun, 63:3621-3627). EC₅₀ values were calculated for the VHH concentration that secreted in a signal equal to 50% of the maximum signal.

### Neutralization Assay

Vero cells (ATCC) at a concentration of 2.4×10⁴ cells/100 μl of medium (DMEM high glucose+1 mM sodium pyruvate, 2 mM L-glutamine, 50 U/ml and 50 μg/ml Pen/Strep pH 7.4 (HyClone)) were plated in 96-well plates overnight for 90-95% confluency, prior to addition of anti-TcdB VHH in serial dilutions (in media) (1:5) or from 100 μg/ml-1.0 fg/ml and toxic levels of TcdB, such as 0.01 ng/ml to 1 ng/ml TcdB, in a 24 hour cytotoxicity/cell rounding assay (Yang, Z. et al., 2014, J Infect Disease, 210(6):964-972; doi:10.1093/infdis/jiu196). After 24 hours, toxicity to the cells was assessed by quantifying the percent (%) of cells in the plates that became rounded in each of the wells. Apparent IC₅₀ values for the anti-TcdB VHH polypeptides were determined.

### Computational Analysis

In general, data are analyzed using GraphPad Prism software version 6. All error bars refer to standard deviations. ELISA data are analyzed using nonlinear regression.

### Animal Experiments

Animal experiments involving the anti-TcdB VHHs as described herein are conducted at the Department of Infectious Disease and Global Health, Tufts Cummings School of Veterinary Medicine (North Grafton, USA) in conformance with Tufts University IACUC Protocol #G2016-74. Six-to eight-week-old female CDI mice (Charles River Labs, Wilmington, USA) are randomized based on body weight and receive single intravenous injections of LNP-formulated mRNA into the tail vein. Blood is sampled by retro-orbital bleeding at defined times, and VHH accumulation in sera is measured as described by J. M. Tremblay et al., 2013, Infec. Immun., 81:4592-4603; Mukherjee et al., 2014, PLoS ONE 9e106422. In all studies, animals are housed under standard and humane conditions with a standard commercial rodent diet and tap water provided to the animals ad libitum.

All experiments involving animals are performed under protocols approved by Tufts University and National Institute of Allergy and Infectious Diseases (NIAID) Animal Care and Use Committees. Work with alpacas was performed at Tufts under approved protocol Tuskegee University School of Veterinary Medicine (TUSVM) and Institutional Animal Care and Use Committee (IACUC) Protocol #G2015-49. Mouse studies are performed at NIAID under approved protocols LPD8E and LPD9E.

### Mouse Systemic Toxin Challenge

For in vivo studies using mice, 6 or 8 week old C57BL/6 or Balb/cJ female mice (Jackson Laboratories, Bar Harbor, ME) are intraperitoneally (IP) injected with a single dose of VHH, e.g., anti-C. diff. TcdB VHH (50 μg/mouse), 1 hour prior to IP injection of TcdB (100 or 200 ng/mouse). Mice are monitored for signs and symptoms of toxemia (including: lethargy, depression, anorexia, dehydration, ruffled coat, and hunched posture), e.g., over a predetermined time period, e.g., 10 days. Moribund mice are euthanized following IACUC-approved removal criteria.

### Mouse CDI Challenge

In vivo experiments using mice are conducted to mimic the human condition of C. difficile infection (CDI) and disease and to facilitate colonization with C. diffiicile. For these experiments, ten 6 week old, C57BL/6 female mice receive filter sterilized antibiotics (kanamycin, gentamycin, colistin, metranidozole, and vancomycin) in drinking water for 5 days followed by 2 days of water alone. After 2 days of drinking the supplemented water, the mice receive one (100 μl) intraperitoneal (IP) injection of clindamycin (2 mg/ml). One day later, mice are orally challenged (Chen, X. et al., 2008, Gastroenterology. 135:1984-1992) with 106 spores of an NAPI/027/BI C. difficile strain, designated strain UK6 (Killgore, G. et al., 2008, J. Clin Microbiol, 46:431-437) only (control group), or are inoculated with spores and administered anti-TcdB VHH (25-50 μg/mouse) or dimeric or multimeric forms thereof, at 4, 24 and 48 hours post-challenge (treated group). Blood is collected at 72, 96 and 120 hours post-challenge to determine VHH titers. Animals administered anti-TcdB VHH are expected to be less prone to C. difficile-associated disease or severe disease, symptoms and outcomes, and to have increased survival (less morbidity and death) compared with control animals that did not receive anti-TcdB VHH. In some experiments, the animals receive a prophylactic dose of anti-TcdB VHH, or dimeric or multimeric forms thereof, prior to inoculation with spores so as to prevent C. difficile-associated disease and/or to reduce the severity of disease and symptoms thereof, and/or to improve survival.

### Hamster CDI Challenge

In vivo experiments using hamsters are conducted to mimic the human condition of C. difficile infection and disease and to facilitate colonization with C. diffiicile. For these experiments, male Golden Syrian hamsters (110-135 g) are administered clindamycin (30 mg/kg) via oral gavage for 5 days prior to oral inoculation with 1000 C. difficile strain UK6 spores. Infected control hamsters are administered clindamycin, are inoculated with UK6 spores and are given sterile PBS, by intraperitoneal (IP) administration, 2 times per day for the duration of the experiment. Anti-TcdB VHH-treated hamsters are administered clindamycin, inoculated with spores and are given purified anti-TcdB VHH (1 mg/kg), or dimeric or multimeric forms thereof, by IP administration, 2 times a day for the duration of the experiment. A blood sample is collected at time of euthanasia for detection of anti-TcdB VHH in serum. Necropsies are performed on euthanized animals and tissues are collected for histopathologic examination. Animals administered anti-TcdB VHH are expected to be less prone to C. difficile-associated disease or severe disease, symptoms and outcomes, and to have increased survival (less morbidity and death) compared with control animals that did not receive anti-TcdB VHH. In some experiments, the animals receive a prophylactic dose of anti-TcdB VHH, or dimeric or multimeric forms thereof, prior to inoculation with spores so as to prevent C. difficile-associated disease and/or to reduce the severity of disease and symptoms thereof, and/or to improve survival.

### Pig CDI Challenge

Pigs have been demonstrated to mimic C. difficile infection, colonization and disease as experienced by humans. For in vivo experiments using pigs, thirty gnotobiotic piglets are derived via Caesarean section and maintained in sterile isolators for the duration of the experiment (Tzipori, S. et al., 1995, Infect Immun, 63:3621-3627). Five groups of piglets are orally inoculated with 106 C. difficile UK6 spores (group 1-5) and group 6 was the uninfected control group. Group 1 (n=3) receives anti-TcdB VHH (1 mg/pig) 4 hours prior to, and Group 2 (n=3) 18 hours post oral inoculation with spores. After the initial dose, the treated groups receive 2 doses of anti-TcdB VHH (1 mg/pig) per day either via IP or intra muscular (IM) administration for the duration of the experiment. The anti-TcdB VHH treated group (Group 3; n=9) is given 1.0×10¹¹ viral particles by IV administration one day prior to oral inoculation with 106 C. difficile UK6 spores and 3 days post infection. Group 4 (n=6) receives buffer as control, given 4 hours prior to oral inoculation with 106 C. difficile UK6 spores and at 24 hour post inoculation, and then every 12 hours until the termination of the experiment. Group 5 is given control adenovirus expressing an unrelated VHH (n=6), (1.0×10¹¹ viral particles) by IV administration one day prior to oral inoculation with 106 C. difficile UK6 spores and 3 days post infection. Group 6 (n=3) is uninfected. Fecal samples are collected from all piglets for bacterial culture, and blood samples are collected 1-3 times (when possible) during the experiment and at the time of euthanasia to determine anti-TcdB VHH titers. Necropsies are performed on all animals and tissues are collected for histopathologic examination. Animals administered anti-TcdB VHH are expected to be less prone to C. difficile-associated disease or severe disease, symptoms and outcomes, and to have increased survival (less morbidity and death) compared with control animals that did not receive anti-TcdB VHH. In some experiments, the animals receive a prophylactic dose of anti-TcdB VHH, or dimeric or multimeric forms thereof, prior to inoculation with spores so as to prevent C. difficile-associated disease and/or to reduce the severity of disease and symptoms thereof, and/or to improve survival.

### Histology

Tissue samples from C. difficile colonized animals are collected during necropsy and preserved in 10% neutral buffered formalin. Formalin fixed samples are embedded in paraffin, sectioned at 5 μm, and stained using hematoxylin and eosin using routine histochemical techniques at TCSVM Histopathology Service Laboratory. Light microscopic examination and lesion evaluation are performed by a board-certified veterinary pathologist (GB) with results reported for severity (minimal, mild, moderate, marked), epithelial ulceration, luminal contents, and quantification (Sponseller, J K et al., 2014, J Infect Dis, doi:10.1093/infdis/jiu605). Briefly, a quantitative assessment of colitis severity is performed by counting neutrophilic foci in colon sections from each sample. Foci are observed between colonic crypts in the lamina propria in 10 random fields with ×20 magnification.

All publications, patents, published patent applications and sequence database entries mentioned and disclosed herein are hereby incorporated by reference in their entireties as if each individual publication or patent were specifically and individually indicated to be incorporated by reference.

1.-104. (canceled)

105. A polypeptide that specifically binds to C. difficile toxin B (TcdB), or a TcdB-binding portion thereof, wherein the polypeptide comprises three complementarity determining regions (CDRs), CDR1, CDR2 and CDR3, and four VHH framework regions (FRs), FR1, FR2, FR3 and FR4, with the general structure FR1-CDR1-FR2-CDR2-FR3-CDR3-FR4,
wherein CDR1 comprises amino acids X1-X8, wherein X₁ is G or E, or a conservative amino acid substitution thereof; X₂ is R or A, or a conservative amino acid substitution thereof; X₃ is G or N, or a conservative amino acid substitution thereof; X₄ is P or L, or a conservative amino acid substitution thereof; X₅ is G, or a conservative amino acid substitution thereof; X₆ is I, or a conservative amino acid substitution thereof; X₇ is N, or a conservative amino acid substitution thereof; and X₈ is V or A, or a conservative amino acid substitution thereof;
wherein CDR2 comprises amino acids X₁-X₇, wherein X₁ is Q, or a conservative amino acid substitution thereof; X₂ is T, or a conservative amino acid substitution thereof; X₃ is G, or a conservative amino acid substitution thereof; X₄ is G, or a conservative amino acid substitution thereof; X₅ is T, R, or S, or a conservative amino acid substitution thereof; X₆ is T or L, or a conservative amino acid substitution thereof; and X₇is N or S, or a conservative amino acid substitution thereof; and
wherein CDR3 comprises amino acids X₁-X₉, wherein X₁ is Y, or a conservative amino acid substitution thereof; X₂ is L or Q, or a conservative amino acid substitution thereof; X₃ is K, or a conservative amino acid substitution thereof; X₄ is K or R, or a conservative amino acid substitution thereof; X₅ is W, or a conservative amino acid substitution thereof; X₆ is R, or a conservative amino acid substitution thereof; X₇ is D or N, or a conservative amino acid substitution thereof; X₈ is E, Q, D, or R, or a conservative amino acid substitution thereof; and X₉ is Y, or a conservative amino acid substitution thereof;
wherein the conservative amino acid substitution constitutes a substitute or replacement amino acid residue having similar or identical charge, hydrophobicity, and/or size.

106. The polypeptide of claim 105, wherein the four VHH FRs are camelid VHH FRs.

107. The polypeptide of claim 105, wherein the polypeptide neutralizes C. difficile toxin B (TcdB) activity.

108. The polypeptide of claim 105, which is a camelid-derived single domain anti-TcdB VHH antibody.

109. The polypeptide of claim 105, in the form of a dimer or multimer polypeptide.

110. The polypeptide of claim 109, wherein the dimer or multimer polypeptide comprises two or more of polypeptides, or a TcdB binding portion thereof, which are joined with one or more linker peptides.

111. The polypeptide of claim 110, wherein the one or more linker peptides is selected from GGGGS as set forth in SEQ ID NO: 63; GGGGSGGGGGGGGS as set forth in SEQ ID NO: 64, or a functional portion thereof; EPKTPKPQGGGGGGGGSGGGGSQGVQSQVQLVE as set forth in SEQ ID NO: 65; or EPKTPKPQ as set forth in SEQ ID NO: 66, or a combination thereof.

112. The polypeptide of claim 109, wherein the dimer or multimer polypeptide comprises one or more epitope tag sequences specifically bindable by an anti-epitope tag antibody or a binding portion thereof.

113. The polypeptide of claim 112, wherein the one or more epitope tag sequences comprises at least one of DELGPRLMGK as set forth in SEQ ID NO: 61 or an E-tag epitope of amino acid sequence GAPVPYPDPLEPR as set forth in SEQ ID NO: 62.

114. A method of treating, preventing, or reducing the severity of C. difficile infection (CDI) or intoxication by C. difficile and/or the symptoms thereof in a subject who has, who is susceptible to, or who is at risk of CDI or intoxication by C. diffiicile, the method comprising administering to a subject in need thereof an effective amount of the polypeptide of claim 105, a dimer or multimer thereof, or a pharmaceutical composition thereof, thereby reducing the severity of C. difficile infection (CDI) or intoxication by C. difficile and/or the symptoms thereof in the subject.

115. The method of claim 114, wherein the polypeptide, the dimer or multimer thereof, or the pharmaceutical composition thereof is administered to the subject prior or subsequent to infection by C. difficile; and optionally, wherein the subject is to receive or has received antibiotics or a course of antibiotics to treat a non-C. difficile infection.

116. The method of claim 114, wherein the CDI and the symptoms thereof cause one or more of C. difficile-associated diarrhea (CDAD), pseudomembranous colitis (PMC), bowel inflammation, enterocytic detachment, alteration, disruption, or elimination of natural intestinal microflora, and/or paralytic ileus.

117. An isolated polynucleotide encoding the polypeptide of claim 105.

118. An isolated polynucleotide encoding the polypeptide of claim 109.

119. A vector comprising the isolated polynucleotide of claim 117.

120. A vector comprising the isolated polynucleotide of claim 118.

121. A host cell comprising the vector of claim 119.

122. A host cell comprising the vector of claim 120.

123. A composition comprising the polypeptide of claim 105.

124. A composition comprising the polypeptide of claim 109.


SEQUENCE LISTING (155 sequences)

SEQ ID NO: 1 - protein, 123 aa, synthetic construct
  REGION 1..123: note = Description of Artificial Sequence: Synthetic polypeptide
QVQLAESGGGLVQAGGSLRLSCAASGSVYTFMGWYRQAPGKTRELVAGISGGTITKYADSVKGRFIISRDSPKNTIYLQMNELKVEDTGVYYCNAGDTIAQAMGTRRFPFDRWGQGTQVTVAS

SEQ ID NO: 2 - DNA, 369 bp, synthetic construct
  misc_feature 1..369: note = Description of Artificial Sequence: Synthetic polynucleotide
  source 1..369: mol_type = other DNA
caggtgcagctcgcggagtcgggaggaggcttggtgcaggctggggggtctctgagactctcctgtgcagcctctggaagcgtgtatacttttatgggctggtaccgccaggctccagggaagacgcgcgaattggtcgcagggatttcaggtggtacgatcacaaaatatgcagactccgtgaagggccgattcatcatctccagagacagccccaagaacacaatctatctgcaaatgaacgagctaaaagttgaagacacaggcgtgtattactgtaatgcaggcgacaccattgcacaggctatggggacgcggaggtttccgttcgaccgctggggccaggggacccaggtcaccgtcgcctca

SEQ ID NO: 3 - protein, 116 aa, synthetic construct
  REGION 1..116: note = Description of Artificial Sequence: Synthetic polypeptide
QVQLAESGGGLVQAGGSLRLSCVGSGTSFPRNYMGWYRQAPGKQRELVAAISHDGNVEYADSVKGRFTISRGNFVNTVALQMNSLKSEDTAVYYCKLVTLRRDEYWGQGTQVTVSS

SEQ ID NO: 4 - DNA, 369 bp, synthetic construct; identical to SEQ ID NO: 2
  misc_feature 1..369: note = Description of Artificial Sequence: Synthetic polynucleotide
  source 1..369: mol_type = other DNA

SEQ ID NO: 5 - protein, 127 aa, synthetic construct
  REGION 1..127: note = Description of Artificial Sequence: Synthetic polypeptide
QLQLVESGGGLVQPGGSLRLSCTSARFSLINYAIGWFRQAPGQKREGVSLLTSGGATYYADSARDRFTISRDNARNTVYLQMNSLKPEDTAVYSCAAGPYSRTLVSRWKVGDGMEYWGKGTLVTVSS

SEQ ID NO: 6 - DNA, 381 bp, synthetic construct
  misc_feature 1..381: note = Description of Artificial Sequence: Synthetic polynucleotide
  source 1..381: mol_type = other DNA
cagttgcagctcgtggagtctgggggaggcctggtgcagcctgggggttctctgagactctcctgtacatctgcgagattctctttgattaattatgccataggctggttccgccaggccccaggacagaagcgcgagggggtctcactacttactagtggtggtgctacatactatgctgactccgcgagggaccgattcaccatctccagagacaacgccaggaacacggtgtatttgcaaatgaacagcctgaaacctgaggacacggccgtttattcgtgtgcagcagggccctattcccgaaccttagtctcacgttggaaggtgggggacggcatggagtactggggcaaagggaccctagtcaccgtctcctca

SEQ ID NO: 7 - protein, 124 aa, synthetic construct
  REGION 1..124: note = Description of Artificial Sequence: Synthetic polypeptide
QVQLAESGGGLVQPGGSLRLSCAASGFTSNSYYIGWFRQAPGKGREAVSSISSSGGSPNYANAVKGRFTITRDNANNTVYLQMDNLKPEDTAVYYCAASKFPLTTMASNRYHYWGQGTQVTVSS

SEQ ID NO: 8 - DNA, 372 bp, synthetic construct
  misc_feature 1..372: note = Description of Artificial Sequence: Synthetic polynucleotide
  source 1..372: mol_type = other DNA
caggtgcagctcgcggagtcgggcggaggcttggtgcagcctgggggttctctgagactctcctgtgcagcctctggattcacttcgaattcttattacataggctggttccgccaggccccagggaaggggcgcgaggcggtctcaagtattagtagtagtggaggtagccctaactatgcgaatgccgtgaagggccgattcaccataaccagagacaacgccaacaacacggtctatctgcaaatggacaacctgaaacctgaggacacggccgtttattactgcgcagcatcgaagtttccgctaaccactatggcgtccaaccgatatcattactggggtcaggggacccaggtcaccgtctcctca

SEQ ID NO: 9 - protein, 114 aa, synthetic construct
  REGION 1..114: note = Description of Artificial Sequence: Synthetic polypeptide
QLQLVETGGLVQAGGSLRLSCVGSGRGPGINVMGWYRQAPGTERELVATWQTGGTTNYADSVKGRFTISRDNLKNTVSLQMDSLKPEDTAVYYCYLKKWRDEYWGQGTQVTVSS

SEQ ID NO: 10 - DNA, 342 bp, synthetic construct
  misc_feature 1..342: note = Description of Artificial Sequence: Synthetic polynucleotide
  source 1..342: mol_type = other DNA
cagttgcagctggtggagacgggaggcttggtgcaggctggggggtctctgagactctcctgtgtaggctctggaagaggccccgggatcaatgtcatgggctggtaccgccaggctccagggactgagcgcgagttggtcgcaacttggcaaaccggtggtaccacaaactatgcagactccgtgaagggccgattcaccatctccagagacaacctcaagaacacggtgtcgctgcaaatggacagtctgaaacctgaggacacagccgtctattactgctatctgaaaaaatggagagatgagtattggggccaggggacccaggtcaccgtctcctca

SEQ ID NO: 11 - protein, 121 aa, synthetic construct
  REGION 1..121: note = Description of Artificial Sequence: Synthetic polypeptide
QVQLVESGGGLAQAGGSLRLSCAASGSSFSMNVMGWYRQPPGKQRELVATIRSDGITNYAESVKGRFTISRDNVKNTVHLEMNRLKAEDTAVYYCFHGRARTGNNADLGSWGQGTQVTVSS

SEQ ID NO: 12 - DNA, 363 bp, synthetic construct
  misc_feature 1..363: note = Description of Artificial Sequence: Synthetic polynucleotide
  source 1..363: mol_type = other DNA
caggtgcagctggtggagtcgggtggaggcttggcgcaggctggggggtctctgagactctcctgtgcagcctctggaagtagcttcagcatgaatgtcatgggctggtaccgccagcctccagggaagcagcgcgagttggtcgcgactattcgtagtgatggtatcacaaactatgcagagtccgtgaagggccgattcaccatctccagagacaacgtcaagaacacagtgcatctggaaatgaacaggctgaaagctgaagacacagccgtatattactgctttcatggccgggcccgcacagggaataatgctgacttgggttcttggggccaggggacccaggtcaccgtctcctcg

SEQ ID NO: 13 - protein, 119 aa, synthetic construct
  REGION 1..119: note = Description of Artificial Sequence: Synthetic polypeptide
QVQLAETGGGLVQAGGSLRLSCAASGRLSERIFMISTMAWYRQVPGKQRELVAEISRLGRANYSDSVTDRFIISRDNTKNTVDLQMNSLKPEDTAVYYCNLKPFVDNYRGPGTQVTVSS

SEQ ID NO: 14 - DNA, 357 bp, synthetic construct
  misc_feature 1..357: note = Description of Artificial Sequence: Synthetic polynucleotide
  source 1..357: mol_type = other DNA
caggtgcagctggcggagacgggtggaggcttggtgcaggctggggggtctctgagactctcctgtgcagcttcgggaaggctctcggaaaggatcttcatgatcagtacgatggcctggtaccgccaggttccagggaagcagcgcgagttggtcgcagaaatttcgcgacttggtagggccaactattcagactccgtgacggaccgattcatcatctccagagacaacaccaagaacacggtggatctacaaatgaacagcctgaagcctgaggacacagccgtctattactgcaatcttaaacccttcgtcgacaactaccggggcccggggacccaggtcaccgtctcctct

SEQ ID NO: 15 - protein, 120 aa, synthetic construct
  REGION 1..120: note = Description of Artificial Sequence: Synthetic polypeptide
QLQLAESGGGLVQPGGSLRLSCAASGITFSNVAMSWVRQAPGKGLEWVSTISTGGSSTSYLDSVKSRFTISRDNAKKTVYLQMNSLKPEDTAVYYCVKGPKYSATIRRPEGQGTQVTVSS

SEQ ID NO: 16 - DNA, 360 bp, synthetic construct
  misc_feature 1..360: note = Description of Artificial Sequence: Synthetic polynucleotide
  source 1..360: mol_type = other DNA
cagttgcagctcgcggagtccggcggaggcttggtgcagcctggggggtctctgagactctcctgtgcagcctctggaatcaccttcagtaacgttgccatgagctgggtccgccaggctccaggaaaggggctcgagtgggtctcaactattagtacgggcggtagtagtacaagctatttagactccgtgaagagccgattcaccatctccagagacaacgccaagaagacggtgtatctgcaaatgaacagtctgaaacctgaggacacggccgtgtattactgtgtaaaagggcccaagtattccgctacaatccgtcgtcctgagggccaggggacccaggtcaccgtctcctca

SEQ ID NO: 17 - protein, 119 aa, synthetic construct
  REGION 1..119: note = Description of Artificial Sequence: Synthetic polypeptide
QLQLVESGGGLVQPGGSLRLSCVASGFNFSVQIMSWVRQAPGKGLEWVSAISTGGASKSYADFAKGRFTISRDNAKNTLYLQMNSLQLEDTAVYFCSKGPRTWINSSPRGQGTQVTVSS

SEQ ID NO: 18 - DNA, 357 bp, synthetic construct
  misc_feature 1..357: note = Description of Artificial Sequence: Synthetic polynucleotide
  source 1..357: mol_type = other DNA
cagttgcagctcgtggagtccggtggaggcttggtgcagcctggggggtctctgagactctcctgtgtagcctctggattcaacttcagtgtgcagattatgagctgggtccgccaggctccaggaaaggggctcgagtgggtctcagctattagtactggtggcgctagcaaaagttatgcagacttcgcgaagggccgattcaccatctccagagacaacgccaagaacacgctgtatctgcaaatgaacagcctacaactcgaggacacggccgtgtatttttgttctaagggtccgaggacttggatcaattctagtccccggggccaggggacccaggtcaccgtctcctcg

SEQ ID NO: 19 - protein, 123 aa, synthetic construct
  REGION 1..123: note = Description of Artificial Sequence: Synthetic polypeptide
QVQLVESGGGLVQPGGSLTINCTVSGTAFSLDTMTWYRQAPGKQRELAADISSSGASNYLASVKGRFTISRDNAKSALYLQMNSLKPEDTGTYYCYRGRVRGVWPLDSGMMYWGKGTLVTVSS

SEQ ID NO: 20 - DNA, 369 bp, synthetic construct
  misc_feature 1..369: note = Description of Artificial Sequence: Synthetic polynucleotide
  source 1..369: mol_type = other DNA
caggtgcagctggtggagtccggtggaggcttggtgcagcctgggggctctctgacaatcaactgtacagtctctggaaccgcgttcagtctcgataccatgacctggtaccgccaggctccagggaagcagcgcgagttggccgccgatattagtagtagtggtgcctcaaactatttagcctccgtgaagggccgattcaccatctccagagataacgccaagagcgctctgtatctgcaaatgaacagcctgaaacctgaggacacaggcacatattattgctatagagggcgagtgcggggagtctggccgttggacagcggcatgatgtactggggtaaagggaccttggtcaccgtctcctca

SEQ ID NO: 21 - protein, 120 aa, synthetic construct
  REGION 1..120: note = Description of Artificial Sequence: Synthetic polypeptide
QVQLVETGGGLVQAGGSLRLSCAASGSILSSMGWYRQAPGNQREFVASISRTGATDYADSVAGRFIISRDRGKNTVLALQMDSLKPEDTAVYYCNAGLGMGDPRRPGPWWGQGTQVTVSS

SEQ ID NO: 22 - DNA, 360 bp, synthetic construct
  misc_feature 1..360: note = Description of Artificial Sequence: Synthetic polynucleotide
  source 1..360: mol_type = other DNA
caggtgcagctcgtggagactggaggaggcttggtgcaggctggggggtctctgagactctcctgtgcagcctctggaagcatcctcagttccatgggctggtaccgccaggctccaggaaaccagcgcgagttcgtcgcgtctatttctcgtactggggcgacggactatgcagactccgtggcgggccgattcatcatctctagggacagaggcaagaacacggtattagcgctgcaaatggacagcctgaaacctgaggacacagccgtctattactgtaatgcaggattaggaatgggggacccgcgacggcccggtccgtggtggggccagggaacccaggtcaccgtctcctca

SEQ ID NO: 23 - protein, 115 aa, synthetic construct
  REGION 1..115: note = Description of Artificial Sequence: Synthetic polypeptide
QVQLAETGGGLVQAGGSLRLSCVSSERNPGINAMGWYRQAPGSQRELVAVWQTGGSLSYADSVKGRFTISRDNLKNTVYLQMNSLKPEDAAVYYCYLKKWRDQYWGQGTRVTVSS

SEQ ID NO: 24 - DNA, 345 bp, synthetic construct
  misc_feature 1..345: note = Description of Artificial Sequence: Synthetic polynucleotide
  source 1..345: mol_type = other DNA
caggtgcagctcgcggagactggtggaggcttggttcaggcaggcgggtctctgagactctcctgtgtaagctctgaaagaaatcccgggatcaatgccatgggctggtatcgccaggctccagggagtcagcgcgagttggtcgcagtttggcaaaccggtggtagcctaagctatgcagactccgtgaagggtcgattcaccatctccagagacaacctcaagaacacggtgtatctgcaaatgaacagtctgaaacctgaggacgcagccgtctattattgttatctgaaaaagtggagagatcagtattggggccaggggacccgggtcaccgtctcctca

SEQ ID NO: 25 - protein, 6 aa, synthetic construct
  REGION 1..6: note = Description of Artificial Sequence: Synthetic peptide
GSVYTF

SEQ ID NO: 26 - protein, 7 aa, synthetic construct
  REGION 1..7: note = Description of Artificial Sequence: Synthetic peptide
SGGTITK

SEQ ID NO: 27 - protein, 19 aa, synthetic construct
  REGION 1..19: note = Description of Artificial Sequence: Synthetic peptide
NAGDTIAQAMGTRRFPFDR

SEQ ID NO: 28 - protein, 8 aa, synthetic construct
  REGION 1..8: note = Description of Artificial Sequence: Synthetic peptide
GTSFPRNY

SEQ ID NO: 29 - protein, 7 aa, synthetic construct
  REGION 1..7: note = Description of Artificial Sequence: Synthetic peptide
SHDGNVE

SEQ ID NO: 30 - protein, 10 aa, synthetic construct
  REGION 1..10: note = Description of Artificial Sequence: Synthetic peptide
KLVTLRRDEY

SEQ ID NO: 31 - protein, 8 aa, synthetic construct
  REGION 1..8: note = Description of Artificial Sequence: Synthetic peptide
RFSLINYA

SEQ ID NO: 32 - protein, 7 aa, synthetic construct
  REGION 1..7: note = Description of Artificial Sequence: Synthetic peptide
TSGGATY

SEQ ID NO: 33 - protein, 21 aa, synthetic construct
  REGION 1..21: note = Description of Artificial Sequence: Synthetic peptide
AAGPYSRTLVSRWKVGDGMEY

SEQ ID NO: 34 - protein, 8 aa, synthetic construct
  REGION 1..8: note = Description of Artificial Sequence: Synthetic peptide
GFTSNSYY

SEQ ID NO: 35 - protein, 8 aa, synthetic construct
  REGION 1..8: note = Description of Artificial Sequence: Synthetic peptide
SSSGGSPN

SEQ ID NO: 36 - protein, 17 aa, synthetic construct
  REGION 1..17: note = Description of Artificial Sequence: Synthetic peptide
AASKFPLTTMASNRYHY

SEQ ID NO: 37 - protein, 8 aa, synthetic construct
  REGION 1..8: note = Description of Artificial Sequence: Synthetic peptide
GRGPGINV

SEQ ID NO: 38 - protein, 7 aa, synthetic construct
  REGION 1..7: note = Description of Artificial Sequence: Synthetic peptide
QTGGTTN

SEQ ID NO: 39 - protein, 9 aa, synthetic construct
  REGION 1..9: note = Description of Artificial Sequence: Synthetic peptide
YLKKWRDEY

SEQ ID NO: 40 - protein, 8 aa, synthetic construct
  REGION 1..8: note = Description of Artificial Sequence: Synthetic peptide
GSSFSMNV

SEQ ID NO: 41 - protein, 7 aa, synthetic construct
  REGION 1..7: note = Description of Artificial Sequence: Synthetic peptide
RSDGITN

SEQ ID NO: 42 - protein, 15 aa, synthetic construct
  REGION 1..15: note = Description of Artificial Sequence: Synthetic peptide
FHGRARTGNNADLGS

SEQ ID NO: 43 - protein, 12 aa, synthetic construct
  REGION 1..12: note = Description of Artificial Sequence: Synthetic peptide
GRLSERIFMIST

SEQ ID NO: 44 - protein, 7 aa, synthetic construct
  REGION 1..7: note = Description of Artificial Sequence: Synthetic peptide
SRLGRAN

SEQ ID NO: 45 - protein, 10 aa, synthetic construct
  REGION 1..10: note = Description of Artificial Sequence: Synthetic peptide
NLKPFVDNYR

SEQ ID NO: 46 - protein, 8 aa, synthetic construct
  REGION 1..8: note = Description of Artificial Sequence: Synthetic peptide
GITFSNVA

SEQ ID NO: 47 - protein, 8 aa, synthetic construct
  REGION 1..8: note = Description of Artificial Sequence: Synthetic peptide
STGGSSTS

SEQ ID NO: 48 - protein, 14 aa, synthetic construct
  REGION 1..14: note = Description of Artificial Sequence: Synthetic peptide
VKGPKYSATIRRPE

SEQ ID NO: 49 - protein, 8 aa, synthetic construct
  REGION 1..8: note = Description of Artificial Sequence: Synthetic peptide
GFNFSVQI

SEQ ID NO: 50 - protein, 8 aa, synthetic construct
  REGION 1..8: note = Description of Artificial Sequence: Synthetic peptide
STGGASKS

SEQ ID NO: 51 - protein, 13 aa, synthetic construct
  REGION 1..13: note = Description of Artificial Sequence: Synthetic peptide
SKGPRTWINSSPR

SEQ ID NO: 52 - protein, 8 aa, synthetic construct
  REGION 1..8: note = Description of Artificial Sequence: Synthetic peptide
GTAFSLDT

SEQ ID NO: 53 - protein, 7 aa, synthetic construct
  REGION 1..7: note = Description of Artificial Sequence: Synthetic peptide
SSSGASN

SEQ ID NO: 54 - protein, 17 aa, synthetic construct
  REGION 1..17: note = Description of Artificial Sequence: Synthetic peptide
YRGRVRGVWPLDSGMMY

SEQ ID NO: 55 - protein, 6 aa, synthetic construct
  REGION 1..6: note = Description of Artificial Sequence: Synthetic peptide
GSILSS

SEQ ID NO: 56 - protein, 7 aa, synthetic construct
  REGION 1..7: note = Description of Artificial Sequence: Synthetic peptide
SRTGATD

SEQ ID NO: 57 - protein, 15 aa, synthetic construct
  REGION 1..15: note = Description of Artificial Sequence: Synthetic peptide
NAGLGMGDPRRPGPW

SEQ ID NO: 58 - protein, 8 aa, synthetic construct
  REGION 1..8: note = Description of Artificial Sequence: Synthetic peptide
ERNPGINA

SEQ ID NO: 59 - protein, 8 aa, synthetic construct
  REGION 1..8: note = Description of Artificial Sequence: Synthetic peptide
WQTGGSLS

SEQ ID NO: 60 - protein, 9 aa, synthetic construct
  REGION 1..9: note = Description of Artificial Sequence: Synthetic peptide
YLKKWRDQY

SEQ ID NO: 61 - protein, 10 aa, synthetic construct
  REGION 1..10: note = Description of Artificial Sequence: Synthetic peptide
DELGPRLMGK

SEQ ID NO: 62 - protein, 13 aa, synthetic construct
  REGION 1..13: note = Description of Artificial Sequence: Synthetic peptide
GAPVPYPDPLEPR

SEQ ID NO: 63 - protein, 5 aa, synthetic construct
  REGION 1..5: note = Description of Artificial Sequence: Synthetic peptide
GGGGS

SEQ ID NO: 64 - protein, 15 aa, synthetic construct
  REGION 1..15: note = Description of Artificial Sequence: Synthetic peptide
GGGGSGGGGSGGGGS

SEQ ID NO: 65 - protein, 34 aa, synthetic construct
  REGION 1..34: note = Description of Artificial Sequence: Synthetic polypeptide
EPKTPKPQGGGGSGGGGSGGGGSQGVQSQVQLVE

SEQ ID NO: 66 - protein, 8 aa, synthetic construct
  REGION 1..8: note = Description of Artificial Sequence: Synthetic peptide
EPKTPKPQ

SEQ ID NO: 67 - protein, 8 aa, synthetic construct
  REGION 1..8: note = Description of Artificial Sequence: Synthetic peptide
DYKDDDDK

SEQ ID NO: 68 - protein, 10 aa, synthetic construct
  REGION 1..10: note = Description of Artificial Sequence: Synthetic His tag
  SITE 1..10: note = This sequence may encompass 5-10 residues
HHHHHHHHHH

SEQ ID NO: 69 - protein, 6 aa, synthetic construct
  REGION 1..6: note = Description of Artificial Sequence: Synthetic 6xHis tag
HHHHHH

SEQ ID NO: 70 - protein, 7 aa, synthetic construct
  REGION 1..7: note = Description of Artificial Sequence: Synthetic peptide
QTGGSLS

SEQ ID NO: 71 - protein, 13 aa, synthetic construct
  REGION 1..13: note = Description of Artificial Sequence: Synthetic peptide
DICLPRWGCLWED

SEQ ID NO: 72 - protein, 25 aa, synthetic construct
  REGION 1..25: note = Description of Artificial Sequence: Synthetic peptide
QVQLAESGGGLVQAGGSLRLSCAAS

SEQ ID NO: 73 - protein, 18 aa, synthetic construct
  REGION 1..18: note = Description of Artificial Sequence: Synthetic peptide
MGWYRQAPGKTRELVAGI

SEQ ID NO: 74 - protein, 37 aa, synthetic construct
  REGION 1..37: note = Description of Artificial Sequence: Synthetic polypeptide
YADSVKGRFIISRDSPKNTIYLQMNELKVEDTGVYYC

SEQ ID NO: 75 - protein, 11 aa, synthetic construct
  REGION 1..11: note = Description of Artificial Sequence: Synthetic peptide
WGQGTQVTVAS

SEQ ID NO: 76 - protein, 25 aa, synthetic construct
  REGION 1..25: note = Description of Artificial Sequence: Synthetic peptide
QVQLAESGGGLVQAGGSLRLSCVGS

SEQ ID NO: 77 - protein, 18 aa, synthetic construct
  REGION 1..18: note = Description of Artificial Sequence: Synthetic peptide
MGWYRQAPGKQRELVAAI

SEQ ID NO: 78 - protein, 37 aa, synthetic construct
  REGION 1..37: note = Description of Artificial Sequence: Synthetic polypeptide
YADSVKGRFTISRGNFVNTVALQMNSLKSEDTAVYYC

SEQ ID NO: 79 - protein, 11 aa, synthetic construct
  REGION 1..11: note = Description of Artificial Sequence: Synthetic peptide
WGQGTQVTVSS

SEQ ID NO: 80 - protein, 25 aa, synthetic construct
  REGION 1..25: note = Description of Artificial Sequence: Synthetic peptide
QLQLVESGGGLVQPGGSLRLSCTSA

SEQ ID NO: 81 - protein, 18 aa, synthetic construct
  REGION 1..18: note = Description of Artificial Sequence: Synthetic peptide
IGWFRQAPGQKREGVSLL

SEQ ID NO: 82 - protein, 37 aa, synthetic construct
  REGION 1..37: note = Description of Artificial Sequence: Synthetic polypeptide
YADSARDRFTISRDNARNTVYLQMNSLKPEDTAVYSC

SEQ ID NO: 83 - protein, 11 aa, synthetic construct
  REGION 1..11: note = Description of Artificial Sequence: Synthetic peptide
WGKGTLVTVSS

SEQ ID NO: 84 - protein, 25 aa, synthetic construct
  REGION 1..25: note = Description of Artificial Sequence: Synthetic peptide
QVQLAESGGGLVQPGGSLRLSCAAS

SEQ ID NO: 85 - protein, 18 aa, synthetic construct
  REGION 1..18: note = Description of Artificial Sequence: Synthetic peptide
IGWFRQAPGKGREAVSSI

SEQ ID NO: 86 - protein, 37 aa, synthetic construct
  REGION 1..37: note = Description of Artificial Sequence: Synthetic polypeptide
YANAVKGRFTITRDNANNTVYLQMDNLKPEDTAVYYC

SEQ ID NO: 87 - protein, 24 aa, synthetic construct
  REGION 1..24: note = Description of Artificial Sequence: Synthetic peptide
QLQLVETGGLVQAGGSLRLSCVGS

SEQ ID NO: 88 - protein, 18 aa, synthetic construct
  REGION 1..18: note = Description of Artificial Sequence: Synthetic peptide
MGWYRQAPGTERELVATW

SEQ ID NO: 89 - protein, 37 aa, synthetic construct
  REGION 1..37: note = Description of Artificial Sequence: Synthetic polypeptide
YADSVKGRFTISRDNLKNTVSLQMDSLKPEDTAVYYC

SEQ ID NO: 90 - protein, 25 aa, synthetic construct
  REGION 1..25: note = Description of Artificial Sequence: Synthetic peptide
QVQLVESGGGLAQAGGSLRLSCAAS

SEQ ID NO: 91 - protein, 18 aa, synthetic construct
  REGION 1..18: note = Description of Artificial Sequence: Synthetic peptide
MGWYRQPPGKQRELVATI

SEQ ID NO: 92 - protein, 37 aa, synthetic construct
  REGION 1..37: note = Description of Artificial Sequence: Synthetic polypeptide
YAESVKGRFTISRDNVKNTVHLEMNRLKAEDTAVYYC

SEQ ID NO: 93 - protein, 25 aa, synthetic construct
  REGION 1..25: note = Description of Artificial Sequence: Synthetic peptide
QVQLAETGGGLVQAGGSLRLSCAAS

SEQ ID NO: 94 - protein, 18 aa, synthetic construct
  REGION 1..18: note = Description of Artificial Sequence: Synthetic peptide
MAWYRQVPGKQRELVAEI

SEQ ID NO: 95 - protein, 37 aa, synthetic construct
  REGION 1..37: note = Description of Artificial Sequence: Synthetic polypeptide
YSDSVTDRFIISRDNTKNTVDLQMNSLKPEDTAVYYC

SEQ ID NO: 96 - protein, 10 aa, synthetic construct
  REGION 1..10: note = Description of Artificial Sequence: Synthetic peptide
GPGTQVTVSS

SEQ ID NO: 97 - protein, 25 aa, synthetic construct
  REGION 1..25: note = Description of Artificial Sequence: Synthetic peptide
QLQLAESGGGLVQPGGSLRLSCAAS

SEQ ID NO: 98 - protein, 18 aa, synthetic construct
  REGION 1..18: note = Description of Artificial Sequence: Synthetic peptide
MSWVRQAPGKGLEWVSTI

SEQ ID NO: 99 - protein, 37 aa, synthetic construct
  REGION 1..37: note = Description of Artificial Sequence: Synthetic polypeptide
YLDSVKSRFTISRDNAKKTVYLQMNSLKPEDTAVYYC

SEQ ID NO: 100 - protein, 10 aa, synthetic construct
  REGION 1..10: note = Description of Artificial Sequence: Synthetic peptide
GQGTQVTVSS

SEQ ID NO: 101 - protein, 25 aa, synthetic construct
  REGION 1..25: note = Description of Artificial Sequence: Synthetic peptide
QLQLVESGGGLVQPGGSLRLSCVAS

SEQ ID NO: 102 - protein, 18 aa, synthetic construct
  REGION 1..18: note = Description of Artificial Sequence: Synthetic peptide
MSWVRQAPGKGLEWVSAI

SEQ ID NO: 103 - protein, 37 aa, synthetic construct
  REGION 1..37: note = Description of Artificial Sequence: Synthetic polypeptide
YADFAKGRFTISRDNAKNTLYLQMNSLQLEDTAVYFC

SEQ ID NO: 104 - protein, 25 aa, synthetic construct
  REGION 1..25: note = Description of Artificial Sequence: Synthetic peptide
QVQLVESGGGLVQPGGSLTINCTVS

SEQ ID NO: 105 - protein, 18 aa, synthetic construct
  REGION 1..18: note = Description of Artificial Sequence: Synthetic peptide
MTWYRQAPGKQRELAADI

SEQ ID NO: 106 - protein, 37 aa, synthetic construct
  REGION 1..37: note = Description of Artificial Sequence: Synthetic polypeptide
YLASVKGRFTISRDNAKSALYLQMNSLKPEDTGTYYC

SEQ ID NO: 107 - protein, 25 aa, synthetic construct
  REGION 1..25: note = Description of Artificial Sequence: Synthetic peptide
QVQLVETGGGLVQAGGSLRLSCAAS

SEQ ID NO: 108 - protein, 18 aa, synthetic construct
  REGION 1..18: note = Description of Artificial Sequence: Synthetic peptide
MGWYRQAPGNQREFVASI

SEQ ID NO: 109 - protein, 38 aa, synthetic construct
  REGION 1..38: note = Description of Artificial Sequence: Synthetic polypeptide
YADSVAGRFIISRDRGKNTVLALQMDSLKPEDTAVYYC

SEQ ID NO: 110 - protein, 25 aa, synthetic construct
  REGION 1..25: note = Description of Artificial Sequence: Synthetic peptide
QVQLAETGGGLVQAGGSLRLSCVSS

SEQ ID NO: 111 - protein, 17 aa, synthetic construct
  REGION 1..17: note = Description of Artificial Sequence: Synthetic peptide
MGWYRQAPGSQRELVAV

SEQ ID NO: 112 - protein, 37 aa, synthetic construct
  REGION 1..37: note = Description of Artificial Sequence: Synthetic polypeptide
YADSVKGRFTISRDNLKNTVYLQMNSLKPEDAAVYYC

SEQ ID NO: 113 - protein, 11 aa, synthetic construct
  REGION 1..11: note = Description of Artificial Sequence: Synthetic peptide
WGQGTRVTVSS

SEQ ID NO: 114 - protein, 25 aa, synthetic construct
  REGION 1..25: note = Description of Artificial Sequence: Synthetic peptide
  MOD_RES 5: note = Q or V
  MOD_RES 11: note = L or S
  MOD_RES 14: note = A or P
QVQLXESGGGXVQXGGSLRLSCAAS

SEQ ID NO: 115 - protein, 20 aa, synthetic construct
  REGION 1..20: note = Description of Artificial Sequence: Synthetic peptide
  MOD_RES 2: note = S or T
  MOD_RES 9: note = A or P
  MOD_RES 14: note = R or T
  MOD_RES 15: note = L or I
  MOD_RES 16: note = S or N
  MOD_RES 18: note = A, V or T
  MOD_RES 19: note = A, G, V or S
  MOD_RES 20: note = S or A
EXGGGLVQXGGSLXXXCXXX

SEQ ID NO: 116 - protein, 14 aa, synthetic construct
  REGION 1..14: note = Description of Artificial Sequence: Synthetic peptide
  MOD_RES 2: note = F, Y or V
  MOD_RES 9: note = E, C or G
  MOD_RES 10: note = R or L
  MOD_RES 12: note = F, G, L or W
  MOD_RES 14: note = A, S or T
WXRQAPGKXXEXVX

SEQ ID NO: 117 - protein, 14 aa, synthetic construct
  REGION 1..14: note = Description of Artificial Sequence: Synthetic peptide
  MOD_RES 2: note = F, Y or V
  MOD_RES 5: note = A or P
  MOD_RES 8: note = K, S or Q
  MOD_RES 9: note = Q, G, E, K or T
  MOD_RES 10: note = R or L
  MOD_RES 12: note = L, W or G
  MOD_RES 13: note = V or D
  MOD_RES 14: note = A or S
WXRQXPGXXXEXXX

SEQ ID NO: 118 - protein, 37 aa, synthetic construct
  REGION 1..37: note = Description of Artificial Sequence: Synthetic polypeptide
  MOD_RES 2: note = A, Q, T or V
  MOD_RES 3: note = D or E
  MOD_RES 5: note = V or A
  MOD_RES 10: note = T or A
  MOD_RES 11: note = I or V
  MOD_RES 13: note = R or Q
  MOD_RES 15: note = N or K
  MOD_RES 17: note = K or A
  MOD_RES 20: note = V, L or M
  MOD_RES 25: note = N or D
  MOD_RES 26: note = S or N
  MOD_RES 28: note = K or R
  MOD_RES 30: note = E or D
  MOD_RES 33: note = A or G
  MOD_RES 34: note = V, I, T or M
YXXSXKGRFXXSXDXAXNTXYLQMXXLXPXDTXXYYC

SEQ ID NO: 119 - protein, 37 aa, synthetic construct
  REGION 1..37: note = Description of Artificial Sequence: Synthetic polypeptide
  MOD_RES 2: note = A, L or S
  MOD_RES 3: note = D, N, A or E
  MOD_RES 4: note = S, F or A
  MOD_RES 5: note = V or A
  MOD_RES 6: note = K, R or T
  MOD_RES 7: note = G, S or D
  MOD_RES 10: note = T or I
  MOD_RES 12: note = S or T
  MOD_RES 14: note = D or G
  MOD_RES 15: note = N or S
  MOD_RES 16: note = A, L, T, V, F or P
  MOD_RES 17: note = K, N or V
  MOD_RES 18: note = N, K or S
  MOD_RES 19: note = T or A
  MOD_RES 20: note = V or L
  MOD_RES 21: note = Y, H, D or S
  MOD_RES 23: note = Q or E
  MOD_RES 25: note = N or D
  MOD_RES 26: note = S, N, R or E
  MOD_RES 28: note = K or Q
  MOD_RES 29: note = P, L, S or V
  MOD_RES 33: note = A or G
  MOD_RES 34: note = V or T
  MOD_RES 36: note = Y, F or S
YXXXXXXRFXIXRXXXXXXXXLXMXXLXXEDTXXYXC

SEQ ID NO: 120 - protein, 123 aa, synthetic construct; identical to SEQ ID NO: 1
  REGION 1..123: note = Description of Artificial Sequence: Synthetic polypeptide

SEQ ID NO: 121 - protein, 109 aa, synthetic construct
  REGION 1..109: note = Description of Artificial Sequence: Synthetic polypeptide
SGGGLVQAGGSLRLSCVGSGRGPGINVMGWYRQAPGTERELVATWQTGGTTNYADSVKGRFTISRDNLKNTVSLQMDSLKPEDTAVYYCYLKKWRDEYWGQGTQVTVSS

SEQ ID NO: 122 - protein, 11 aa, synthetic construct
  REGION 1..11: note = Description of Artificial Sequence: Synthetic peptide
  MOD_RES 1: note = W or R
  MOD_RES 3: note = Q, K or P
  MOD_RES 6: note = Q or L
  MOD_RES 10: note = S or A
XGXGTXVTVXS

SEQ ID NO: 123 - protein, 117 aa, synthetic construct
  REGION 1..117: note = Description of Artificial Sequence: Synthetic polypeptide
  MOD_RES 37: note = Any amino acid
  MOD_RES 45: note = Any amino acid
  MOD_RES 83: note = Any amino acid
SGGGLVQAGGSLRLSCAASGTVYTFMGWYRHPPSKTXQLDSGIYXGTIANYAYSENGQVLFSIYRRNNTIYLHMNEIKLEDTXVYYCYASYTIAQAMGTRRFPIHRWGPGNPVTVAS

SEQ ID NO: 124 - protein, 109 aa, synthetic construct
  REGION 1..109: note = Description of Artificial Sequence: Synthetic polypeptide
SGGGLVQAGGSLRLSCATSGANLGINAMGWYRQAPGSQRELVATWQTGGSTNYADSVKGRATISRDTAKNTVNLQMNSLKPEDTAVYYCYLKRWRNDYWGQGTQVTVYS

SEQ ID NO: 125 - protein, 108 aa, synthetic construct
  REGION 1..108: note = Description of Artificial Sequence: Synthetic polypeptide
TGGLVQAGGSLRLSCVGSGRGPGINVMGWYRQAPGTERELVATWQTGGTTNYADSVKGRFTISRDNLKNTVSLQMDSLKPEDTAVYYCYLKKWRDEYWGQGTQVTVSS

SEQ ID NO: 126 - protein, 109 aa, synthetic construct
  REGION 1..109: note = Description of Artificial Sequence: Synthetic polypeptide
TGGGLVQAGGSLRLSCVSSERNPGINAMGWYRQAPGGQRELVAVWQTGGSLNYADSVKGRFTISRDNLKNTVYLQMNSLKPEDTAVYYCYLKKWRDQYWGQGTQVTVSS

SEQ ID NO: 127 - protein, 109 aa, synthetic construct
  REGION 1..109: note = Description of Artificial Sequence: Synthetic polypeptide
TGGGLVQAGGSLRLSCVSSERNPGINAMGWYRQAPGSQRELVAVWQTGGSLNYADSVKGRFTISRDNLKNTVYLQMNSLKPEDTAVYYCYLKKWRDQYWGQGTQVTVSS

SEQ ID NO: 128 - protein, 108 aa, synthetic construct
  REGION 1..108: note = Description of Artificial Sequence: Synthetic polypeptide
TGGGLVQAGGSLRLSCVSSERNPGINAMGWYRAPGSQRELVAVWQTGGSLNYADFVKGRFTISRDNLKNTVYLQMNSLKPEDTAVYYCYLKKWRDQYWGQGTRVTVSS

SEQ ID NO: 129 - protein, 109 aa, synthetic construct
  REGION 1..109: note = Description of Artificial Sequence: Synthetic polypeptide
SGGGLVQAGGSLRLSCVGSGRNPGINAMGWYRQAPGSQRELVAVWQTGGSTNYADSVKGRFTISRDNLKNTVYLQMNSLKPEDTAVYYCYLKKWRDEYWGQGTQVTVSS

SEQ ID NO: 130 - protein, 109 aa, synthetic construct
  REGION 1..109: note = Description of Artificial Sequence: Synthetic polypeptide
TGGGLVQAGGSLRLSCVGSGRNPGINAMGWYRQAPGSQRELVAVWQTGGSTNYADSVKGRFTISRDNLKNTVYLQMNSLKPEDTAVYYCYLKKWRDEYWGQGTQVTVSS

SEQ ID NO: 131 - protein, 109 aa, synthetic construct
  REGION 1..109: note = Description of Artificial Sequence: Synthetic polypeptide
TGGGLVQAGGSLRLSCVGSGRNPGINAMGWYRQAPGSQRELVAVWQTGGRTNYADSVKGRFTISRDNLKNTVYLQMNSLKPEDTAVYYCYLKKWRDEYWGQGTQVTVSS

SEQ ID NO: 132 - protein, 114 aa, synthetic construct
  REGION 1..114: note = Description of Artificial Sequence: Synthetic polypeptide
SGGGLVQPGGSLRLSCAASGITFSNVAMSWVRQAPGKGLEWVSTISTGGSSTSYLDSVKSRFTISRDNAKKTVYLQMNSLKPEDTAVYYCVKGPKYSATIRRPEGQGTQVTVSS

SEQ ID NO: 133 - protein, 114 aa, synthetic construct
  REGION 1..114: note = Description of Artificial Sequence: Synthetic polypeptide
SGGGLVQPGGSLRLSCAASGFTFSNVAMSWVRQAPGKGLEWVSTISTGGTSTAYLDSVKGRFTIFRDNAKNTAYLQMNSLKPEDTAMYYCANQPKALSTISRPKGQGTQVTVSS

SEQ ID NO: 134 - protein, 113 aa, synthetic construct
  REGION 1..113: note = Description of Artificial Sequence: Synthetic polypeptide
TGGGSVQIGGSLRLSCVTSGFTFSKNIMSWVRQAPGKGLEWVSTISIGGAATSYADSVKGRFTISRDNANDTLYLQMNDLKPEDTAVYYCSRGPRTYINTASRGQGTQVTVST

SEQ ID NO: 135 - protein, 114 aa, synthetic construct
  REGION 1..114: note = Description of Artificial Sequence: Synthetic polypeptide
SGGGLVQPGGSLRLSCAASGFTFSKNIMSWVRQAPGKGLEWVSAISIGGVSTSYADSVKGRFTISRDNAKNTLYLQMNSLKPEDTAVYFCVKGPRSASGNSDNRGKGTLVTVSS

SEQ ID NO: 136 - protein, 113 aa, synthetic construct
  REGION 1..113: note = Description of Artificial Sequence: Synthetic polypeptide
TGGGLVQPGGSLRLSCVASGFTFSVQIMSWVRQAPGKGLEWVSTISNGGVSTSYADSAKGRFTISRDNAKNTLYLQMNSLKPEDTAVYYCLKGPPSWINSASRGQGTQVTVSS

SEQ ID NO: 137 - protein, 113 aa, synthetic construct
  REGION 1..113: note = Description of Artificial Sequence: Synthetic polypeptide
  MOD_RES 46: note = Any amino acid
SGGGLVQPGGSLRLSCVASGFNFSVQIMSWVRQAPGKGLEWVSAIXTGGASKSYADFAKGRFTISRDNAKNTLYLQMNSLQLEDTAVYFCSKGPRTWINSSPRGQGTQVTVSS

SEQ ID NO: 138 - protein, 121 aa, synthetic construct
  REGION 1..121: note = Description of Artificial Sequence: Synthetic polypeptide
SGGGLVQPGGSLRLSCTSARFSLINYAIGWFRQAPGQKREGVSLLTSGGATYYADSARDRFTISRDNARNTVYLQMNSLKPEDTAVYSCAAGPYSRTLVSRWKVGDGMEYWGKGTLVTVSS

SEQ ID NO: 139 - protein, 118 aa, synthetic construct
  REGION 1..118: note = Description of Artificial Sequence: Synthetic polypeptide
SGGGLVQPGGSLRLSCAASGFTSNSYYIGWFRQAPGKGREAVSSISSSGGSPNYANAVKGRFTITRDNANNTVYLQMDNLKPEDTAVYYCAASKFPLTTMASNRYHYWGQGTQVTVSS

SEQ ID NO: 140 - protein, 116 aa, synthetic construct
  REGION 1..116: note = Description of Artificial Sequence: Synthetic polypeptide
GGGLVQAGGSLTINCTVSGTAFSLDTMTWYRQAPGKQRELVADISSSGASNYLASVKGRFTISRDNAKSALYLQMNSLKPEDTGTYYCYRGRVRGVWPLDSGMMYWGKGTLVTVSS

SEQ ID NO: 141 - protein, 113 aa, synthetic construct
  REGION 1..113: note = Description of Artificial Sequence: Synthetic polypeptide
TGGGLVQAGGSLRLSCAASGRLSERIFMISTMAWYRQVPGKQRELVAEISRLGRANYSDSVTDRFIISRDNTKNTVDLQMNSLKPEDTAVYYCNLKPFVDNYRGPGTQVTVSS

SEQ ID NO: 142 - protein, 115 aa, synthetic construct
  REGION 1..115: note = Description of Artificial Sequence: Synthetic polypeptide
SGGGLAQAGGSLRLSCAASGSSFSMNVMGWYRQPPGKQRELVATIRSDGITNYAESVKGRFTISRDNVKNTVHLEMNRLKAEDTAVYYCFHGRARTGNNADLGSWGQGTQVTVSS

SEQ ID NO: 143 - protein, 110 aa, synthetic construct
  REGION 1..110: note = Description of Artificial Sequence: Synthetic polypeptide
SGGGLVQAGGSLRLSCAASGNSYARNYMGWFRQAPGKQRELVAEITPAGDTNYAHFVKGRFTTSRGNAVNTVSLQMNSLEPEDTAVYYCKLVDLRRNAYWGQGTQVTVSS

SEQ ID NO: 144 - protein, 110 aa, synthetic construct
  REGION 1..110: note = Description of Artificial Sequence: Synthetic polypeptide
SGGGLVQAGGSLRLSCVGSGTSFPRNYMGWYRQAPGKQRELVAAISHDGNVEYADSVKGRFTISRGNFVNTVALQMNSLKSEDTAVYYCKLVTLRRDEYWGQGTQVTVSS

SEQ ID NO: 145 - protein, 110 aa, synthetic construct
  REGION 1..110: note = Description of Artificial Sequence: Synthetic polypeptide
SGGDLVQAGGSLRLSCVGSGTSFPRNYMGWYRQTPGKQRELVADITRDDNTNYADSVKGRFTISRGNAVNTVHLQMNSLKAEDTAVYYCKLATLRGDQYWGQGTQVTVSS

SEQ ID NO: 146 - protein, 117 aa, synthetic construct
  REGION 1..117: note = Description of Artificial Sequence: Synthetic polypeptide
SGGGLVQAGGSLRLSCAASGSVYTFMGWYRQAPGKTRELVAGISGGTITKYADSVKGRFIISRDSPKNTIYLQMNELKVEDTGVYYCNAGDTIAQAMGTRRFPFDRWGQGTQVTVAS

SEQ ID NO: 147 - protein, 109 aa, synthetic construct
  REGION 1..109: note = Description of Artificial Sequence: Synthetic polypeptide
SGGGLVQAGGSLRLSCVSSERNPGINAMGWYRQAPGSQRELVAVWQTGGSLNYADSVKGRFTISRDNLKNTVYLQMNSLKPEDTAVYYCYLKKWRDQYWGQGTQVTVSS

SEQ ID NO: 148 - protein, 109 aa, synthetic construct
  REGION 1..109: note = Description of Artificial Sequence: Synthetic polypeptide
SGGGLVQAGGSLRLSCVGSGRNPGINAMGWYRQAPGSQRELVAVWQTGGRTNYADSVKGRFTISRDNLKNTVYLQMNSLKPEDTAVYYCYLKKWRDEYWGQGTQVTVSS

SEQ ID NO: 149 - protein, 109 aa, synthetic construct
  REGION 1..109: note = Description of Artificial Sequence: Synthetic polypeptide
SGGGLVQAGGSLRLSCVSSERNPGINAMGWYRQAPGSQRELVAVWQTGGSLNYADFVKGRFTISRDNLKNTVYLQMNSLKPEDTAVYYCYLKKWRDQYWGQGTRVTVSS

SEQ ID NO: 150 - protein, 109 aa, synthetic construct
  REGION 1..109: note = Description of Artificial Sequence: Synthetic polypeptide
SGGGLVQAGGSLRLSCVSSERNPGINAMGWYRQAPGGQRELVAVWQTGGSLNYADSVKGRFTISRDNLKNTVYLQMNSLKPEDTAVYYCYLKKWRDQYWGQGTQVTVSS

SEQ ID NO: 151 - protein, 109 aa, synthetic construct
  REGION 1..109: note = Description of Artificial Sequence: Synthetic polypeptide
SGGGLVQAGGSLRLSCVSSERNPGINAMGWYRQAPGSQRELAAVWQTGGSLNYADSVKGRFTISRDNLKNTVYLQMNSLKPEDTAVYYCYLKKWRDQYWGQGTQVTVSS

SEQ ID NO: 152 - protein, 109 aa, synthetic construct
  REGION 1..109: note = Description of Artificial Sequence: Synthetic polypeptide
SGGGLVQAGGSLRLSCVSSERNPGINAMGWYRQAPGSQRELVAVWQTGGSLNYADSVKGRFTISRDNLKNTVYLQMNSLKPEDTAVYYCYQKKWRDQYWGQGTQVTVSS

SEQ ID NO: 153 - protein, 109 aa, synthetic construct
  REGION 1..109: note = Description of Artificial Sequence: Synthetic polypeptide
SGGGLVQAGGSLRLSCVSSERNPGINAMGWYRQAPGSQRELVAVWQTGGSLNYADSVKGRFTISRDNLKNTVYLQMNSLKPEDTAVYYCYLKKWRDRYWGQGTQVTVSS

SEQ ID NO: 154 - protein, 109 aa, synthetic construct
  REGION 1..109: note = Description of Artificial Sequence: Synthetic polypeptide
SGGGLVQAGGSLRLSCVSSERNPGINAMGWYRQAPGSQRELVAVWQTGGSLSYADSVKGRFTISRDNLKNTVYLQMNSLKPEDAAVYYCYLKKWRDQYWGQGTRVTVSS

SEQ ID NO: 155 - protein, 109 aa, synthetic construct
  REGION 1..109: note = Description of Artificial Sequence: Synthetic polypeptide
TGGGLVQAGGSLRLSCVSSERNPGINAMGWYRQAPGSQRELVAVWQTGGSLSYADSVKGRFTISRDNLKNTVYLQMNSLKPEDAAVYYCYLKKWRDQYWGQGTRVTVSS